(19) United States
(12) Patent Application Publication
(10) Pub. No.: US 2013/0046257 A1
(43) Pub. Date: Feb. 21, 2013

(54) METHODS OF USING AGENTS THAT MODULATE CLAUDIN EXPRESSION

(71) Applicants: Lisa A. Beck, Rochester, NY (US); Anna De Benedetto, Rochester, NY (US)

(72) Inventors: Lisa A. Beck, Rochester, NY (US); Anna De Benedetto, Rochester, NY (US)

(73) Assignee: UNIVERSITY OF ROCHESTER, Rochester, NY (US)

(21) Appl. No.: 13/576,968

(22) PCT Filed: Feb. 7, 2011
(86) PCT No.: PCT/US2011/023950

§ 371 Date: Oct. 25, 2012

Related U.S. Application Data
(60) Provisional application No. 61/302,022, filed on Feb. 5, 2010.

(51) Int. Cl. A61M 35/00; A61P 37/04; A61K 39/00; A61K 38/20

(52) U.S. Cl. 604/307; 424/85.2

(57) ABSTRACT

This invention relates to the use of agents that modulate claudin-1 and/or -23 expression or activity for purposes of regulating tight junction (“TJ”) formation among keratinocytes that express claudin-1 and/or -23, or other cell types that over- or under-express claudin-1 and/or -23. Several aspects of the present invention relate to increasing claudin-1 and/or -23 expression or function to enhance tight junction formation among claudin-1 and/or -23 expressing cells, particularly keratinocyte and antigen presenting cells. Several other aspects of the invention relate to decreasing claudin-1 and/or -23 expression or function to diminish tight junction formation among claudin-1 and/or -23 expressing cells, particularly keratinocytes and antigen presenting cells. Transdermal drug and vaccine formulations including agent(s) that decrease -1 and/or -23 expression or function to diminish tight junction formation among claudin-1 and/or -23 expressing cells, as well as a trans dermal patch for delivering the same, are also included.

This application claims benefit of U.S. Provisional Patent Application Ser. No. 61/302,022, filed Feb. 5, 2010, which is hereby incorporated by reference in its entirety.

This invention was made with support from the National Institutes of Health under grant NO1 AI 400029. The U.S. government has certain rights in this invention.

### FIELD OF THE INVENTION

This invention relates to modulating claudin-1 and/or -23 expression to influence tight junctions for delivery of transdermal vaccine or drug formulations, to inhibit cutaneous pathogen infection, to promote barrier function in individuals having reduced claudin-1 and/or -23 expression, and to promote wound healing.

### BACKGROUND OF THE INVENTION

The skin is the only epithelial surface that has two barrier structures: the stratum corneum (“SC”) and tight junctions (“TJ”). Elias, P. M., “Skin Barrier Function,” Curr. Allergy Asthma Rep. 8:299-305 (2008). It is widely accepted in Atopic Dermatitis (“AD”) subjects that the SC is dysfunctional as the result of one or more of the following defects:
1) reduced levels of SC lipids (Imokawa, G., “Lipid Abnormalities in Atopic Dermatitis,” J. Am. Acad. Dermatol. 45:S29-32 (2001); Murata et al., “Abnormal Expression of Sphingomyelin Acylase in Atopic Dermatitis: an Etiologic Factor for Ceramide Deficiency?” J. Invest. Dermatol. 106:1242-9 (1996); and Pilgram et al., “Aberrant Lipid Organization in Stratum Corneum of Patients With Atopic Dermatitis and Lamellar Ichthyosis,” J. Invest. Dermatol. 117:710-7 (2001));
2) acquired or genetic defects in filaggrin (Palmer et al., “Common Loss-of-Function Variants of the Epidermal Barrier Protein Filaggrin Are a Major Predisposing Factor for Atopic Dermatitis,” Nat. Genet. 38:441-6 (2006); Cork et al., “New Perspectives on Epidermal Barrier Dysfunction in Atopic Dermatitis: Gene-Environment Interactions,” J. Allergy Clin. Immunol. 118:3-21 (2006); and Howell et al., “Cytokine Modulation of Atopic Dermatitis Filaggrin Skin Expression,” J. Allergy Clin. Immunol. 124:R7-R12 (2009)) or other epidermal differentiation proteins;
3) acquired or genetic defects in proteases and/or antiproteases (Cork et al., “New Perspectives on Epidermal Barrier Dysfunction in Atopic Dermatitis: Gene-Environment Interactions,” J. Allergy Clin. Immunol. 118:3-21; quiz 2-3 (2006) and Vasilopoulos et al., “A Nonsynonymous Substitution of Cystatin A, a Cysteine Protease Inhibitor of House Dust Mite Protease, Leads to Decreased mRNA Stability and Shows a Significant Association With Atopic Dermatitis,” Allergy 62:514-9 (2007)); and/or
4) simply the consequence of the physical trauma from widespread scratching that predates the development of all skin lesions.

TJ function as the “gate” for passage of water, ions and solutes through the paracellular pathway. Schluter et al., “The Different Structures Containing Tight Junction Proteins in Epidermal and Other Stratified Epithelial Cells, Including Squamous Cell Metaplasia,” Eur. J. Cell. Biol. 86(11-12):645-55 (2007). TJ also regulate the localization of apical and basolateral membrane components. Whether epidermal TJ have this cell polarity function is still debated. Umeda et al., “Z0-1 and ZO-2 Independently Determine Where Claudins Are Polymerized in Tight-Junction Strand Formation,” Cell 126:741-54 (2006). Some investigators have speculated that TJ might regulate the lipid components found in the SC. Niessen, C. M., “Tight Junctions/Adherens Junctions: Basic Structure and Function,” J. Invest. Dermatol. 127:2525-32 (2007) and Leyvraz et al., “The Epidermal Barrier Function is Dependent on the Serine Protease CAP1/Prss8,” J. Cell Biol. 170:487-96 (2005). This has promoted the notion that these two epidermal barrier structures interact in a dynamic way to ensure that the skin is in fact a formidable barrier. The structure and function of keratinocyte TJ remains an area of active investigation.

TJ are composed of a number of transmembrane proteins such as the claudin family, junctional adhesion molecule (JAM)-A, occludin, and tricellulin. In addition, several scaffolding proteins such zonulae occludens (ZO)-1, ZO-2, ZO-3, multi-PDZ domain protein (MUPP)-1, membrane-associated guanylate kinase (MAGI) and cingulin have been identified in the TJ cytosolic plaque. Niessen, C. M., “Tight Junctions/Adherens Junctions: Basic Structure and Function,” J. Invest. Dermatol. 127:2525-32 (2007). Claudins are four-transmembrane-spanning proteins that determine TJ resistance and permeability and include over 24 members. Tsukita et al., “Multifunctional Strands in Tight Junctions,” Nat. Rev. Mol. Cell. Biol. 2:285-93 (2001); Tsukita and Furuse, “Claudin-Based Barrier in Simple and Stratified Cellular Sheets,” Curr. Opin. Cell Biol. 14:531-6 (2002); and Van Itallie and Anderson, “Claudins and Epithelial Paracellular Transport,” Annu. Rev. Physiol. 68:403-29 (2006). Based on in vitro experiments claudins have been divided into those that increase Trans Epithelial Electric Resistance (“TEER”) or enhance barrier, which includes claudin-1 and -4, and those that reduce TEER and therefore disrupt barrier function, which includes claudins-2 and -6. Utech et al., “Tight Junctions and Cell-Cell Interactions,” Methods Mol. Biol. 341:185-95 (2006).

Although the existence of TJ-like structures in the epidermis has been suggested for some time (Elias and Friend, “The Permeability Barrier in Mammalian Epidermis,” J. Cell Biol. 65:180-91 (1975)), the functional relevance of these structures has been addressed only recently. Tsukita and Furuse, “Claudin-Based Barrier in Simple and Stratified Cellular Sheets,” Curr. Opin. Cell Biol. 14:531-6 (2002); Schluter et al., “Sealing the Live Part of the Skin: The Integrated Meshwork of Desmosomes, Tight Junctions and Curvilinear Ridge Structures in the Cells of the Uppermost Granular Layer of the Human Epidermis,” Eur. J. Cell Biol. 83:655-65 (2004); and Pummi et al., “Epidermal Tight Junctions: ZO-1 and Occludin Are Expressed in Mature, Developing, and Affected Skin and In Vitro Differentiating Keratinocytes,” J. Invest. Dermatol. 117:1050-8 (2001). A major breakthrough came in 2002, when Furuse et al., reported that claudin-1-deficient mice died within 24 hr of birth with wrinkled skin, severe dehydration and increased epidermal permeability as measured by dye studies and transepidermal water loss (TEWL). Furuse et al., “Claudin-Based Tight Junctions Are Crucial for the Mammalian Epidermal Barrier: A Lesson From Claudin-1-Deficient Mice,” J. Cell Biol. 156:1099-111 (2002). Importantly, these mice had no abnormalities in the expression of stratum corneum proteins (e.g., loricrin, involucrin, transglutaminase-1, or Klf4) or lipids that might explain the severe skin phenotype. Although this mouse model established the importance of claudin-1 in skin barrier function, very little is currently known about the role of claudin-1 (or TJ) in human skin diseases or wound healing.

The present invention is directed to overcoming these and other limitations in the art.

### SUMMARY OF THE INVENTION

A first aspect of the invention relates to a transdermal vaccine formulation. The formulation includes a pharmaceutically suitable carrier, an effective amount of an antigen or antigen-encoding nucleic acid molecule present in the carrier, optionally one or more adjuvants, and an amount of IL-17 that is sufficient to transiently disrupt claudin-1 and/or -23 function within tight junctions.

A second aspect of the invention relates to a transdermal drug formulation. The drug formulation includes a pharmaceutically suitable carrier, an effective amount of a therapeutic agent, and an amount of IL-17 that is sufficient to transiently disrupt claudin-1 and/or -23 function within tight junctions.

A third aspect of the invention relates to a transdermal patch that includes a transdermal drug or vaccine formulation according to the present invention.

Strikingly reduced expression of the TJ proteins claudin-1 and -23 only in AD was observed, which was validated at the mRNA and protein levels. Claudin-1 expression inversely correlated with Th2 biomarkers. It was observed that a remarkable impairment of the bioelectric barrier function in AD epidermis. In vitro, it was confirmed that silencing claudin-1 expression in human keratinocytes diminishes TJ function while enhancing keratinocyte proliferation. Finally, CLDN1 haplotype-tagging single nucleotide polymorphisms revealed associations with AD in two North American populations. Taken together, these data demonstrate that an impaired epidermal TJ is a novel feature of skin barrier dysfunction and immune dysregulation observed in AD, and that CLDN1 is a new susceptibility gene in this disease.

The expression/function of the tight junction protein, claudin-1, was evaluated in epithelium from AD and nonatopic (“NA”) subjects and two American populations were screened for single nucleotide polymorphisms (“SNPs”) in claudin-1 (“CLDN1”). In summary, the accompanying examples demonstrate that (1) claudin-1 plays a critical role in human epidermal tight junction function and keratinocyte proliferation, (2) claudin-1 is significantly reduced in nonlesional skin of AD compared to NA and psoriasis subjects, (3) claudin-1 levels are inversely correlated with Th2 biomarkers suggesting that reductions in this key TJ barrier protein may affect the character of the immune response to environmental allergens, and (4) analysis of claudin-1 haplotype-tagging SNPs in two North American populations revealed associations with AD. A role for claudin-23 (“CLDN23”) in TJ function was also identified. In addition, the accompanying examples demonstrate that the susceptibility of AD subjects to widespread cutaneous infections with pathogens (e.g., HSV-1) is related to epidermal barrier defects. In particular, it is shown both mechanistic and genetic results that implicate tight junctions as a critical barrier structure, showing that reductions in claudin-1 levels may promote the spread of epidermal pathogen infections. Collectively, this exemplary data highlights a role for TJ proteins in cutaneous host defense and provides convincing evidence for new therapeutic strategies including local disruption of TJ formation with transdermal drug and vaccine formulations and promoting TJ function formation to prevent pathogen infection and enhance barrier function in susceptible individuals.

### BRIEF DESCRIPTION OF THE DRAWINGS

FIGS. 1A-D are a bottom perspective (FIG. 1A) and cross sectional (FIGS. 1B, 1C, and 1D) views of one embodiment of a transdermal patch according to the present invention.

FIGS. 2A-D illustrate that intercellular junction proteins are dysregulated in normal appearing skin from atopic dermatitis (AD) subjects as compared to non-atopic control subjects(NA). FIG. 2A is a table showing Z ratios from gene arrays performed on nonlesional epithelium. An NP-2 negative pressure vacuum apparatus is shown in FIG. 2B and in use in FIG. 2C. Resulting suction-blister roofs are shown in FIG. 2D. CLDN-1 and -23 were reduced, while the gap junction proteins connexin-26 [GJB2] and connexin-62 [GJA10] were upregulated (shown in FIG. 2A as shaded in the Gap Junctions and Tight Junctions rows). Genes indicative of de-differentiation were either unaffected or increased (shown in FIG. 2A as shaded in the Differential Markers Row).

FIGS. 3A-B are heat map (FIG. 3A) and table (FIG. 3B) representations of the epidermal differentiation complex (“EDC”) genes found on chromosome locus 1q21. These results show that 1q21 are differentially expressed in nonlesional epidermis of AD subjects. FIG. 3A is a graphical representation of gene arrays that were performed on nonlesional epithelium (blister roofs) from Atopic Dermatitis subjects with extrinsic disease (AD; n=5) and non-atopic controls (NA; n=5). Each column represents a single array experiment on a single epidermal sample. A heat map of EDC genes was generated where dark red indicates a gene that is more upregulated than a light red gene in AD vs. NA; while dark green indicates a more down regulated gene as compared to a light green gene that is slightly down regulated. In grayscale, darker shades identify greater deviation than lighter shades. Details concerning up- or down-regulation are provided in FIG. 3B, with Z ratios comparing AD to NA samples.

FIGS. 4A-B are graphs showing the reduced expression of claudin-1 and claudin-23 and enhanced expression of connexin-26 in atopic dermatitis subjects confirmed in additional epidermal samples. FIG. 4A shows graphical results of qPCR performed on blister roofs from AD and NA subjects, which showed a significant reduction in claudin-1 (CLDN1) in AD compared to NA (*P=0.03) and enhanced expression of connexin-26 (GJB2; P=0.03). FIG. 4B shows graphical results of claudin-23 (CLDN23) expression, which was significantly reduced in AD samples compared to controls (**P=0.001). The AD subjects (n=6) recruited for these additional validation experiments had extrinsic disease (male/female=2/4; 34±6 yrs; Total IgE=883.6±443.5 kIU/L) and the NA (n=6) had normal IgE values (male/female=2/4; 32±2 yrs; Total IgE=13.7±9.2 kIU/L).

FIGS. 5A-E show that claudin-1 expression is markedly reduced in AD epidermis. FIGS. 5A and 5B are images of immunolabeled skin biopsies which show reduced claudin-1 immunoreactivity in nonlesional AD skin (n=11) (FIG. 5A) compared to FIG. 5B which is an image of nonatopic, healthy-appearing skin (n=12). Red (shown as darker color in the image) indicates positive immunostaining (Bar=100 μm). FIGS. 5C and 5D are immunofluorescent confocal microscopy images. Using a FITC-conjugated secondary antibody, claudin-1 was shown to have a membranous pattern in both AD (FIG. 5C) and NA (FIG. 5D). The signal intensity was significantly reduced in FIG. 5C (AD epidermis). Positive staining is indicated by green (Bar=20 μm) (shown as lighter color in the image). The dotted line denotes the epidermal-dermal junction. FIG. 5E is a graph showing semiquantitative scoring, which confirmed reduced epidermal expression of claudin-1 in AD (1.3±0.3) compared to NA (2.9±0.1; *P<0.0004).

FIGS. 6A-B are graphs showing that the AD epidermis has altered bioelectric properties compared to NA. FIG. 6A is a graph of the Ussing chamber measurements, which reveal a markedly reduced resistance (92±22.0 Ohms×cm²; n=4) in AD epithelium compared to NA (827±173.3 Ohms×cm²; P=0.01; n=4). This was reflected in an increased permeability to FITC-conjugated albumin in AD (445±24.25 O.D./cm²/h; n=4) compared to NA (175±68.37 O.D./cm²/h; P=0.02; n=4). FIG. 6B is a graph showing results of dilution potential studies, which noted the preservation of membrane selectivity in NA subjects, with Na⁺ ions relatively more permeable than Cl⁻ (0.77±0.03 fold PCl/PNa). In contrast, the selectivity was completely lost in AD epidermis (1.1±0.02 fold PCl/PNa; P=0.001; n=3/group) and both ions were equally permeable.

FIGS. 7A-B are graphs showing that the claudin-1 expression from the gene arrays correlates with Th2 biomarkers (e.g. serum total IgE and total eosinophil counts). The line of FIG. 7A represents the linear least square fit for log₂ CLDN1 expression level vs. log₁₀ total serum IgE (Pearson product-moment correlation coefficient n=14; r=−0.718, P=0.0038). FIG. 7B shows the plot of log₂ CLDN1 expression level vs. log₁₀ total eosinophil count (n=14; r=−0.761, P=0.0016). The disease phenotypes (psoriasis (“PS”), atopic dermatitis, and nonatopic) are denoted by unique symbols.

FIGS. 8A-D show that claudin-1 colocalizes with other tight junction (“TJ”) proteins at the cell membrane in differentiated keratinocytes and this coincides with maximal TJ function. FIG. 8A shows confocal microscopy images that demonstrate in Ca²⁺-differentiated keratinocytes (Hi Ca) that claudin-1 (top row) colocalizes with occludin (middle row) at the cell membrane (see Merge row) (Bars=50 μm). FIG. 8B is a graph of Ca²⁺-induced PHK differentiation results, showing TEER is only observed in differentiated PHK and peaks about 40 h after the increase in Ca²⁺ (Representative of n=5). FIG. 8C is a graph of sodium fluorescein flux, showing that paracellular diffusion of 0.02% sodium fluorescein is markedly reduced in differentiated PHK (3.1±0.6 fold; *P=0.046; n=3). FIG. 8D are images of Western Blots that show enhanced expression of claudin-1 when PHK were differentiated for 48 h in presence of IL-4 (50 ng/ml) and IL-13 stimulation (50 ng/ml) or both cytokines (Representative of n=3).

FIGS. 9A-C show claudin-1 protein is expressed in primary human keratinocytes (“PHK”) mainly when PHK were differentiated in high (1.9 mM; Hi Ca) versus low (0.3 mM; Lo Ca) Ca⁺² containing media for 24 h after confluency. Western blot (FIG. 9A) and densitometry (FIG. 9B) confirmed immunostaining data with higher expression in differentiated PHK (28.8±2.3 pixels) as compared to undifferentiated PHK (8.4±3.6 pixels; *P=0.05; n=4). FIG. 9C are confocal microscopy images showing claudin-1 (shown brighter in Claudin-1 row) colocalizes with ZO-1 (shown lighter in ZO-1 row) at the cell membrane only in the Hi Ca cells (see Merge row). Interestingly, in undifferentiated keratinocytes claudin-1 immunoreactivity is faint and primarily nuclear (shown in second column, Lo Ca), while ZO-1 is already detectable on the membrane with a partially continuous pattern (Bars=50 μm).

FIG. 10 is a table showing the CLDN1 polymorphisms and minor allele frequencies (“MAF”) in two American cohorts who self-report as either European American and African American race.

FIG. 11 is a graph showing evidence for CLDN1 association with risk of atopic dermatitis, early onset AD, and disease severity in two North American populations. The X-axis represents the physical position for each of 27 CLDN1 SNPS shown in relationship to the exonic structure of the CLDN1 gene on chromosome 3q28-q29. The Y-axis denotes the association test result as −log (P-value) corresponding to representative symbols for each of the phenotypes. The standard cutoff for significance (P=0.05) is shown as a horizontal solid line. Outcomes included risk of AD (diamond), early age of onset (<5 years; triangle) and the clinical scoring system called EASI (circle) in African American (AA; blue/shown as darker symbols in FIG. 11) and European American (EA; green/shown as lighter symbols in FIG. 11) populations.

FIGS. 12A-E illustrate results showing that silencing claudin-1 reduces Trans Epithelial Electric Resistance (TEER), increases paracellular permeability, and enhances cell proliferation. FIG. 12A shows images of Western blots that demonstrate a dose-dependent reduction of claudin-1 with CLDN1 siRNA (48 h). No change was observed for occludin. FIG. 12B shows confocal microscopy images of immunostaining, which confirmed claudin-1 expression, but not occludin, was reduced in differentiated PHK following transfection with CLDN1 siRNA (Bars=50 μm). FIGS. 12C-12E are graphs showing claudin-1 knockdown significantly reduced TEER (control: 164±18.2 and CLDN1 siRNA: 80.6±6.4; *P=0.007; n=4) (FIG. 12C), increased sodium fluorescein permeability (control: 27.6±11.7 and CLDN1 siRNA: 52.5±9.2; **P=0.026; n=4) (FIG. 12D), and enhanced cell proliferation as assessed by Click-iT™ EdU assay (**P=0.002; n=3) (FIG. 12E).

FIG. 13 is a graph of results from an experiment measuring expression of other proteins upon silencing claudin-1. These results show that silencing of claudin-1 does not affect expression of other proteins relevant for barrier function, except for connexin-26 (GBJ2) which was upregulated. Cldn1 siRNA (100 nM) resulted in a 50% reduction in claudin-1 transcripts compared to control transfected cells (0.5%±0.06 fold; **P=0.5×10⁻⁶; n=5/group). There was no effect on mRNA expression of occludin, Z0-1, nectin-1, E-cadherin or filaggrin (n=5/group). Connexin-26 (GJB2) was significantly upregulated in claudin-1 siRNA transfected PHK (1.7±0.8 fold; *P=0.05; n=6). Primer sequences used for qPCR are listed in Table 1. Relative gene expressions were calculated by using the 2^{-ΔΔcT} method, in which Ct indicates cycle threshold, the fractional cycle number where the fluorescent signal reaches detection threshold. The normalized Ct value of each sample was calculated using GAPDH as an endogenous control gene.

FIGS. 14A-B illustrate that silencing CLDN-1 enhances cell proliferation. This may be responsible for the greater epidermal thickness observed in nonlesional AD skin. FIG. 14A shows immunostaining images that demonstrate silencing claudin-1 enhances cell proliferation as compared to control siRNA (Representative image of n=3 experiments). Cells were stained with an anti-claudin-1 Ab and a FITC-conjugated secondary (green). Red indicates EdU positive cells (active DNA synthesis) and DAPI (blue) nuclear stain provides some assessment of cell density (Bars=50 μm). As negative control, cells from the same population were not treated with EdU. FIG. 14B is a graph illustrating epidermal area measurements, which shows that epidermis from AD nonlesional skin biopsies (n=12) is thicker than NA controls (n=9; *P=0.05).

FIGS. 15A-E show silencing of claudin-1 in human primary keratinocytes increases Herpes Simplex Virus (HSV)-1 infectivity and spreading. FIGS. 15A and 15B are confocal microscopy images showing HSV-1 immunostaining. The light/bright color indicates HSV-1 staining. The DAPI (darker color) nuclear stain provides an assessment of cell density (Bars=200 μm). Larger and more numerous FFU were detected in PHK treated with CLDN1 siRNA (FIG. 15A) as compared to the control siRNA (FIG. 15B) (Representative images of n=6 experiments). Cells stained with a rabbit IgG isotype control were all negative. For each sample, six random fields were captured at identical acquisition settings and analyzed computationally to objectively quantify differences in Focal Forming Units (FFU). A FFU was defined as a cluster of 3 or more adjacent HSV-1 positive cells. FIG. 15C is a graph showing that an average of 4.8±0.7 FFU/field were counted in CLDN1 knockdown PHK and 2.5±0.5 FFU/field in the control samples (*P=0.05; n=6). FIG. 15D is a graph showing the diameter of the major axis of FFU was significantly greater in PHK whose CLDN1 expression was reduced (207±21 μm) compared to controls (152±40 μm; *P=0.03; n=6). FIG. 15E is a graph showing that similar observations were noted in FFU area (*P=0.04; n=6) in CLDN1 knockdown (15649±4367 pixels) as compared to control transfection (9902±4943 pixels).

FIG. 16 is a graph showing results of an infectious center assay. Infectious center assay demonstrates that CLDN1 knockdown PHK were more infected with HSV-1 (38±6.43%; n=3) than control transfected cells (28±7%; n=3; **P=0.003).

FIG. 17 is a graph showing results of CLDN1 siRNA knockdown on nectin-1 and CLDN-1 expression. CLDN1 siRNA (100 nM) resulted in a 50% reduction in CLDN1 transcripts compared to control transfected cells (0.5%±0.6 fold; *P=0.5×10⁻⁶; n=5/group). There was no effect on mRNA expression of nectin-1 (PVRL1; cldn-1 siRNA: 0.99±0.16 and control: 1.1±0.14; n=5/group), a known HSV-1 binding receptor. Relative gene expressions were calculated by using the 2^{-ΔΔCt} method, in which Ct indicates cycle threshold, the fractional cycle number where the fluorescent signal reaches detection threshold. The normalized Ct value of each sample was calculated using GAPDH.

FIG. 18 is a graph showing evidence for CLDN1 SNP (single nucleotide polymorphism) association with risk of Eczema herpeticum (EH) in atopic dermatitis in two American populations, African American (AA; blue/shown as darker symbols in FIG. 18) and European American (EA; green/shown as lighter symbols in FIG. 18). The X-axis represents the physical position for each of 27 CLDN1 SNPS shown in relationship to the exonic structure of the CLDN1 gene on chromosome 3q28-q29. The Y-axis denotes the association test result as −log (P-value) corresponding to representative symbols for each of the phenotypes. The standard cutoff for significance (P=0.05) is shown as a horizontal solid line. In the European American group, an intronic SNP (rs3774032; OR=0.59 [0.35-0.98] P=0.037) was marginally associated with EH (shown as lighter open square in FIG. 18). When excluding subjects with a FLG mutation, the association became slightly more significant (OR=0.44 [0.22-0.85], P=0.0225; shown as lighter diamond in FIG. 18). Interestingly, one additional intronic SNP emerged (rs3732923, OR=1.93 [1.21-3.07], P=0.0010; shown as lighter diamond in FIG. 18). In the African American population, a SNP (rs3954259; P=0.040; shown as darker square symbol in FIG. 18), in the promoter region was associated with EH, and this association was enhanced when subjects with FLG mutations (P=0.026; shown as diamond symbol in FIG. 18) were excluded.

FIGS. 19A-C are results of TEER measurements in PHK stimulated with S. aureus-derived peptidoglycan (“PGN”) (FIG. 19A), Malp-2 (FIG. 19B), and Pam3CSK4 (a Toll-like Receptor ½ ligand) (FIG. 19C).

FIG. 20 is a graph of results showing S. aureus-derived PGN increases TJ mRNA expressions in PHK. The mRNA expression of tight junction molecules (CLDN1, CLDN2, CLDN4, CLDN23, occludin, ZO-1), gap junction molecules (connexin-26, connexin-43), adherens junction molecules (nectin-1, E-cadherin), and desmosome molecules (DSG-1, DSG-3) were quantified by qPCR before and at two timepoints (4 and 24 hr) after stimulation with PGN (20 μg/ml) in PHK (n=4-7). The dotted line represents expression levels for the control group (media alone) at each timepoint. *p<0.05; **p<0.01

FIGS. 21A-C are images of western blots showing S. aureus-derived PGN and TLR2 ligand increase TJ protein expressions in PHK. PHK were treated with PGN (0, 0.2, 2, 20 μg/ml) (FIG. 21A), Pam3CSK4 (0, 0.1, 1, 20 μg/ml) (FIG. 21B), MALP2 (0, 10, 100, 1000 ng/ml) (FIG. 21C) for 48 hr and CLDN1, occludin, ZO-1 and CLDN23 protein levels were detected from whole cell lysates by Western blot. Quantitative protein expression was determined by densitometry of bands and normalization to the housekeeping protein (β-actin). (Representative blot of n=2-3 experiments).

FIG. 22 is a graph showing results of PHK treatment with a PPARγ agonist ciglitazone (CIG 5 μM). These results demonstrate that treatment with PPARγ agonist increases CLDN1 mRNA (1.7 fold over DMSO alone) and occludin mRNA (2.0 fold) expression in PHK differentiated in high Calcium media (DMEM) for 24 h.

FIG. 23 is a graph showing results of sodium decanoate (SC; 1 mM) reduced TEER in human epidermis sheet. After 4 hours of treatment, TEER was reduced 0.74-fold over media alone; and after 24 hours TEER was reduced 0.54-fold.

FIG. 24 is a graph showing results of PHK treatment with IL-17 (at 100 ng/ml). These results show that IL-17 treatment reduced CLDN1 mRNA expression in PHK differentiated in high Calcium media (DMEM). Reduced expression of CLDN1 was observed at each time point between 24 and 96 h. FIG. 24 shows a representative experiment of n=2.

### DETAILED DESCRIPTION OF THE INVENTION

The present invention involves the use of agents that modulate claudin-1 and/or -23 expression or activity for purposes of regulating tight junction (“TJ”) formation among keratinocytes that express claudin-1 and/or -23, or other cell types that over- or under-express claudin-1 and/or -23, such as antigen presenting cells (e.g., dendritic cells and Langerhans cells) (Kubo et al., “External Antigen Uptake by Langerhans Cells with Reorganization of Epidermal Tight Junction Barriers,” J. Exp. Med. 206:2937-46 (2009), which is hereby incorporated by reference in its entirety). Several aspects of the present invention relate to increasing claudin-1 and/or -23 expression or function to enhance tight junction formation among claudin-1 and/or -23 expressing cells, particularly keratinocyte and antigen presenting cells. Several other aspects of the invention relate to decreasing claudin-1 and/or -23 expression or function to diminish tight junction formation among claudin-1 and/or -23 expressing cells, particularly keratinocytes and antigen presenting cells.

Dysfunction of keratinocyte TJ could explain many of the consequences of a defective skin barrier. For example, increased transepidermal water loss (TEWL), which is a well-established measure of skin barrier integrity and is notably elevated in both lesional and nonlesional skin of AD subjects, is still not readily attributable to FLG mutations. Werner and Lindberg, “Transepidermal Water Loss in Dry and Clinically Normal Skin in Patients With Atopic Dermatitis,” Acta Derm. Venereol. 65:102-5 (1985); Hon et al., “Comparison of Skin Hydration Evaluation Sites and Correlations Among Skin Hydration, Transepidermal Water Loss, SCORAD Index, Nottingham Eczema Severity Score, and Quality of Life in Patients with Atopic Dermatitis,” Am. J. Clin. Dermatol. 9:45-50 (2008); Nemoto-Hasebe et al., “Clinical Severity Correlates With Impaired Barrier in Filaggrin-Related Eczema,” J. Invest. Dermatol. 129:682-9 (2009); and Fallon et al., “A Homozygous Frameshift Mutation in the Mouse Flg Gene Facilitates Enhanced Percutaneous Allergen Priming,” Nat. Genet. 41:602-8 (2009), which are hereby incorporated by reference in their entirety. Thus, other genetic or acquired defects in the skin barrier such as defects in TJ likely explain increased TEWL and the resulting dry skin or xerosis that characterizes AD. Defects in TJ may also explain the increased skin surface pH and decreased Stratum corneum hydration characteristic of AD. Seidenari et al., “Objective Assessment of the Skin of Children Affected by Atopic Dermatitis: A Study of pH, Capacitance and TEWL in Eczematous and Clinically Uninvolved Skin,” Acta Derm Venereol. 75(6):429-33 (1995), which is hereby incorporated by reference in its entirety. Defective structure and function of TJ could also have immunological consequences. For example, Kubo et al. recently demonstrated that activated Langerhans cell, the resident antigen-presenting cell in the epidermis, gain access to foreign antigens by sending dendrites out through epidermal TJ. Kubo et al., “External Antigen Uptake by Langerhans Cells With Reorganization of Epidermal Tight Junction Barriers,” J. Exp. Med. 206:2937-46 (2009), which is hereby incorporated by reference in its entirety. It seems likely that Langerhans cell and other antigen presenting cells will be more likely to sample surface antigens and allergens when epidermal TJ are compromised. This coupled with the recent evidence that Langerhans cells are dendritic cells specialized to induce the differentiation of naïve CD4⁺ T cells to Th2 cells strongly supports the notion that a breach in TJ is likely a critical feature in the initiation of AD. Klechevsky et al., “Functional Specializations of Human Epidermal Langerhans Cells and CD14+ Dermal Dendritic Cells,” Immunity 29:497-510 (2008), which is hereby incorporated by reference in its entirety.

One aspect of the present invention relates to a method of inhibiting pathogen infection or local spread of infection in the skin. The method comprises providing an agent that increases claudin-1 and/or -23 expression in keratinocytes and applying to a region of skin on an individual that is susceptible to pathogen infection an amount of the agent that is effective to increase claudin-1 and/or -23 expression in keratinocytes present in the contacted region of skin, whereby increased claudin-1 and/or -23 expression promotes enhancement of tight junction function and thereby renders the contacted region less susceptible to pathogen infection or local spread of infection.

Agents that can increase claudin-1 and/or -23 expression include, without limitation, interleukins, growth factors, synthetic or naturally occurring peptidoglycans (PGNs), toll-like receptor (TLR) ligands, pathogenic bacteria toxins or avirulence proteins, and peroxisome proliferator-activated receptor (“PPAR”) agonists.

In one embodiment, claudin-1 and/or -23 expression is increased by a suitable PPAR agonist. In one embodiment, the agonist is a PPARα or PPARγ agonist. PPARγ agonists are agents that bind to PPARγ and activate receptor-activated pathways. The PPARγ agonists can optionally have dual activity on other PPAR receptors (PPARα and PPARδ). Exemplary PPARγ agonists include, without limitation, cyclopentenone class prostaglandins, thiazolidinediones, glitazones, lysophosphatidic acid (“LPA”) or LPA derivatives (McIntyre et al., “Identification of an intracellular receptor for lysophosphatidic acid (LPA): LPA is a transcellular PPAR gamma agonist,” Proc. Natl. Acad. Sci. USA 100:131-136; (2003), which is hereby incorporated by reference in its entirety), tyrosine-based agonists, indole-derived agonists, and combinations thereof. A preferred member of the cyclopentenone class of prostaglandins is 15D-prostaglandin J₂. Preferred thiazolidinediones and/or glitazones include, without limitation, ciglitazone, troglitazone, pioglitazone, rosiglitazone, SB213068 (Smith Kline Beecham), GW1929, GW7845 (Glaxo-Wellcome), and L-796449 (Merck). Suitable tyrosine-based agonists include N-(2-benzylphenyl)-L-tyrosine compounds (Henke et al., “N-(2-benzylphenyl)-L-tyrosine PPARgamma Agonists: Discovery of a Novel Series of Patent Antihyperglycemic and Antihyperlipidemic Agents,” J. Med. Chem. 41:5020-5036 (1998), which is hereby incorporated by reference in its entirety. Suitable indole-derived agonists include those disclosed, e.g., in Hanks, et al., “Synthesis and Biological Activity of a Novel Series of Indole-derived PPARgamma Agonists,” Biorg. Med. Chem LLH. 9(23):3329-3334 (1999), which is hereby incorporated by reference in its entirety. Any other PPARγ agonists, whether now known or hereafter developed, can also be utilized in accordance with the present invention.

In addition to the use of PPARγ agonists per se, inducers of PPARγ agonists can also be utilized in accordance with the present invention. Inducers of PPARγ agonists are agents that induce an increase in the expression or production of a native PPARγ agonist. Exemplary inducers of PPARγ agonists include, without limitation, decorin or fragments thereof, enzymes that catalyze formation of prostaglandin D₂ precursor, and combinations thereof. Decorin is a small chondroitin/dermatan sulphate proteoglycan that binds the cytoline transforming growth factor beta (TGF-β) through its core protein. Preferred enzymes that catalyze formation of prostaglandin D₂ precursor are hematopoietic prostaglandin-D synthase and a lipocalin-form prostaglandin-D synthase. Any other inducers of PPARγ agonists, whether now known or hereafter developed, can also be utilized in accordance with the present invention.

PPAR-α agonists may also be used in accordance with the present invention and refers to compounds which activate PPARα. Examples include, but are by no means limited to, WY-14643, clofibrate, benzafibrate, fenofibrate, GW409544 and BM-17.0744.

In yet a further embodiment, claudin-1 and/or -23 expression is increased by an agent other than a PPAR agonist.

Any of a number of suitable interleukins can be used to practice the present invention. Exemplary interleukins that can be used include, without limitation, IL-4, IL-13, IL-25, and IL-33.

Any of a number of suitable growth factors can be used to practice the present invention. Exemplary growth factors include, without limitation, epithelial growth factor (EGF), amphiregulin and transforming growth factor (TGF).

Any of a number of suitable TLR ligands can be used to practice the present invention. Exemplary TLR ligands that can be used include, without limitation, PAM3CSK4 (a synthetic triacylated lipopeptide, TLR2/TLR1 ligand), PAM2CSK4 (a synthetic diacylated lipoprotein-TLR2/TLR6 ligand), Poly I:C (a synthetic TLR3 ligand), MALP-2 and FSL-1 (Pam2CGDPKHPKSF). MALP-2, macrophage-activating lipopeptide-2, is induced via TLR2 and TLR6 signaling. FSL-1 is a synthetic lipoprotein derived from Mycoplasma salivarium similar to MALP-2, an M. fermentans derived lipopeptide (LP).

Any of a number of suitable PGNs can be used to practice the present invention. Exemplary PGNs include, without limitation, naturally occurring full-length peptidoglycan (PGN), muramyl dipeptide (MDP, a NOD2 ligand), O—(N-acetyl-β-D-glucosaminyl)-(1→4)-N-acetylmuramyl-L-alanyl-D-isoglutamine, O—(N-acetyl-β-muramyl-L-alanyl-D-isoglutamine)-(1→4)-N-acetyl-D-glucosamine, meso-diaminopimelic acid (meso-DAP), glucosaminyl-N-acetyl)-β-(1→4)-(anhydro)muramyl-N-acetyl-L-alanyl-γ-D-glutaminyl-meso-DAP-D-alanine, glucosaminyl-N-acetyl)-β-(1→4)-muramyl-N-acetyl-L-alanyl-γ-D-glutaminyl-meso-DAP-D-alanine, MurNAc-L-Ala-D-isoGln-L-Lys-D-Ala-D-Ala (MPP), MurNAc-L-Ala-D-isoGln-L-Lys-D-Ala, MurNAc-L-Ala-D-isoGln-(2S,6R)-Dap-D-Ala-D-Ala (MPP-Dap), GlcNAc-MurNAc(1,6-anhydro)-L-Ala-D-isoGlu-(2S,6R)-Dap-D-Ala (TCT), GlcNAc-MurNAc-L-Ala-D-isoGln-L-Lys-D-Ala)₂ (T-4P₂), and various PGN derivatives described in Wolfert et al., “Modification of the Structure of Peptidoglycan Is a Strategy To Avoid Detection by Nucleotide-Binding Oligomerization Domain Protein 1,” Infection and Immunity, 75(2):706-713 (2007), PCT Application Publ. No. WO 2006/113792, US Application Publ. No. 20090214598, and US Application Publ. No. 20070041986, each of which is hereby incorporated by reference in its entirety.

Any of a number of suitable pathogenic bacteria toxins or avirulence proteins can be used in practicing the present invention. Exemplary toxins include, without limitation, Vibrio cholera zonula occludens toxin (Zot) and active fragments thereof (e.g., AT1002) (Song et al., “Effect of the six-mer peptide (AT1002) fragment of zonula occludens toxin on the intestinal absorption of cyclosporine A,” Int. J. Pharm. 351:8-14 (2008), which is hereby incorporated by reference in its entirety). Exemplary avirulence proteins include, without limitation, Salmonella AvrA (see PCT Application Publ. No. WO 2009/149191, which is hereby incorporated by reference in its entirety).

In carrying out the method of inhibiting pathogen infection or local spread of infection in the skin, the region of skin to be treated is generally any region of skin that is susceptible to pathogen infection. By way of example, the region of skin may include at least a portion of the individual's hand, foot, face, or genitalia. Other regions of exposed skin can also be treated in accordance with the present invention. Application of the compositions can be carried out as described above, preferably as part of a daily routine (i.e., after bathing) to inhibit virus infection or local spread thereof. In the case of HSV reactivation it may be used prior to increased sun exposure when he HSV infection typically reactivates in sun-exposed regions of the body.

The pathogen targeted according to the present invention may be a virus. Any virus that is transmitted or infects via the epidermis can be targeted by the methods of the present invention. Exemplary viruses whose infection can be inhibited or blocked include, without limitation, HIV-1, vaccinia virus, varicella zoster virus, herpes simplex viruses (HSV), papillomavirus (e.g., HPV), molluscum contagiosum or Variola (Smallpox) or monkeypox.

The pathogen targeted according to the present invention may be a bacterial pathogen. Any bacteria that is transmitted or infects via the epidermis can be targeted by the methods of the present invention. Exemplary bacterial pathogens whose infection can be inhibited or blocked include, but are not limited to, infections caused by gram-positive and gram-negative bacteria including Staphylococcus, Staphylococcus aureus (including MRSA and MSSA), Hemophilus, Hemophilus influenzae, Pseudomonas, Pseudomonas aeruginosa, Streptococcus, Streptococcus pneumoniae, Streptococcus Group A, Group B, Group C, Group D, Group G, Mycobacterium, Mycobacterium tuberculosis, Atypical Mycobacterium, Clostridium, and Enterobacteriaceae.

According to one embodiment, individuals to be treated for inhibiting pathogen infection or local spread of infection can be healthy individuals having normal TJ protein function.

According to another embodiment, individuals to be treated for inhibiting pathogen infection or local spread of infection can be individuals that have compromised TJ protein function, particular with respect to claudin-1 and/or -23 expression levels or activity. Individuals that have compromised TJ protein function can include, without limitation, those that are identified as having atopic dermatitis (AD, or eczema), psoriasis, contact dermatitis, drug eruptions, Darier's Disease, Netherton's Syndrome, Hyper IgE syndrome, Wiskott Aldrich syndrome, neonatal sclerosing cholangitis associated with ichthyosis, or two or more of the above. By virtue of their compromised TJ function, these individuals are particularly susceptible to virus infection, as demonstrated in the accompanying examples for AD patients and HSV.

Another aspect of the invention relates to a method of enhancing epidermal barrier formation in a patient having a skin wound that extends to the dermis. The method comprises introducing a skin graft or tissue scaffold onto a site of dermal disruption of a subject and applying to the treated site an amount of agent that increases claudin-1 and/or -23 expression in keratinocytes present at the site, thereby promoting more rapid tight junction formation and enhancing epidermal barrier formation at the site.

In one embodiment the site of dermal disruption is treated with an agent selected from a PPARγ agonist, a PPARα agonist, or a combination thereof.

In yet a further embodiment, the site of dermal disruption is treated with an agent other than a PPAR agonist, including interleukins, growth factors, synthetic or naturally occurring peptidoglycans (PGNs), toll-like receptor (TLR) ligands, and pathogenic bacteria toxins or avirulence proteins listed above

Regions of the skin having reduced epithelial function may include any region of injury, which communicates with the atmosphere, by direct exposure. The skin site may be intact (e.g., normal skin) or may be compromised, defined as skin that is damaged or that lacks at least some of the stratum corneum (e.g., skin damaged by exposure to the agent in question, another agent, the presence of a pathological condition such as a rash or contact dermatitis, a physical trauma such as a cut, wound, or abrasion, a underdeveloped skin such as occurs in a preterm infant, conditions in which either all or part of the epidermis is exposed, conditions in which part of the dermis has been removed such as partial thickness wounds encountered in resurfacing procedures such as chemical peels, dermabrasions, and laser resurfacing, etc.).

Open wounds or denudated areas are also included. Open wounds include, but are not limited to, decubital ulcers, dehiscence wounds, acral lick dermatitis (acral lick granulomas in animals), lacerations, and both traumatic and surgical wounds. By ulcer or cutaneous ulcer it is meant a break in the continuity of the epidermis with a loss of substance and exposure of underlying tissue. The region may also include a burn wound, which may include a surface wound ranging from first to third degree burn and ranging from affecting 0.1% to 99.9% body surface area. Exemplary regions include, but are not limited to, those regions disrupted by burn (e.g., thermal or chemical), cutaneous ulcer, severe Stevens-Johnson Syndrome, toxic epidermal necrolysis, autoimmune blistering disorders, or those that having a region of denudation.

Any skin graft or tissue scaffold or heterologous or autologous epidermal sheets suitable for re-epithelialization may be used in accordance with the present invention. Exemplary skin grafts include any natural skin substitutes such as xenografts, allografts, and autografts. Exemplary tissue scaffolds include, but are not limited to, epidermal sheets, collagen-based matrices, natural polymers (e.g., chitosan, fibrin, elastin, gelatin, and hyaluronic acid), synthetic polymer scaffolds, and electrospun biomimetic nanofibrous scaffolds. Zhong et al., “Tissue Scaffolds for Skin Wound Healing and Dermal Reconstruction,” WIREs Nanomedicine and Nanotechnology 2:510-525 (2010), which is hereby incorporated by reference in its entirety. The tissue scaffold may be in any suitable form including, but not limited to, that of a gel, sheet, lattice or sponge. The scaffold may also be formed so as to include the agent that induces claudin-1 and/or -23 expression or function. This will allow the agent to be released at the site of scaffold use where it can affect tight junction formation between keratinocytes. The scaffold may also include or be administered with skin cells (e.g., keratinocytes, fibroblasts, or both).

Yet another aspect of the present invention relates to a method of promoting epithelial function in an individual having compromised or immature epithelial function. The method involves providing an agent that enhances tight junction formation between keratinocytes and administering the agent to a region of skin on an individual having compromised or immature epithelial function at the region, wherein the individual is an infant or is an individual of any age that has a defect in skin barrier or a genetic or acquired condition for which TJ defects are a component of the disease. Examples of such conditions are noted above.

According to one embodiment, the agent is applied to the region of skin up to several times daily. According to another embodiment, the agent is applied to the region of skin once daily. According to further embodiments, the agent is applied to the region of skin periodically (e.g., every other or every third day).

The individual having compromised or immature epithelial function includes a full-term infant, a preterm infant, a low-birth-weight infant, or a very-low-birth-weight infant. As used herein, the terms “preterm” or “preterm infant” may include low-birth-weight infants or very-low-birth weight infants. Low-birth-weight infants are those born from about 32 to about 37 weeks of gestation or weighing from about 3.25 to about 5.5 pounds at birth. Very-low-birth-weight infants are those born before about 32 weeks of gestation or weighing less than about 3.25 pounds at birth. Thus, preterm infants may include infants born before about 37 weeks gestation and/or those weighing less than about 5.5 pounds at birth.

According to one embodiment, the agent is applied to the compromised or immature skin up to several times daily. According to another embodiment, the agent is applied to the compromised or immature skin once daily.

In one embodiment the individual having compromised or immature epithelial function is treated with a PPARγ agonist, a PPARα agonist, a PPARδ agonist, or a combination thereof.

In yet a further embodiment, the individual having compromised or immature epithelial function is treated with an agent other than a PPAR agonist.

As noted above, other aspects of the invention involve the use of agents that decrease claudin-1 and/or -23 expression or function.

Agents that can decrease claudin-1 and/or -23 expression include, without limitation, antisense nucleic acid molecules, including interfering RNA molecules (RNAi), certain interleukins, and fatty acid agents.

An important feature of RNAi affected by siRNA is the double stranded nature of the RNA and the absence of large overhanging pieces of single stranded RNA, although dsRNA with small overhangs and with intervening loops of RNA has been shown to effect suppression of a target gene. In this specification, it will be understood that in this specification the terms siRNA and RNAi are interchangeable. Furthermore, as is well-known in this field, RNAi technology may be carried out by siRNA, miRNA or shRNA or other RNAi inducing agents. Although siRNA will be referred to in general in the specification. It will be understood that any other RNA inducing agent may be used, including shRNA, miRNA or an RNAi-inducing vector whose presence within a cell results in production of an siRNA or shRNA targeted to a target claudin-1 and/or -23 transcript.

RNA interference is a multistep process and is generally activated by double-stranded RNA (dsRNA) that is homologous in sequence to the targeted claudin-1 and/or -23 gene. Introduction of long dsRNA into the cells of organisms leads to the sequence-specific degradation of homologous gene transcripts. The long dsRNA molecules are metabolized to small (e.g., 21-23 nucleotide (nt)) interfering RNAs (siRNAs) by the action of an endogenous ribonuclease known as Dicer. The siRNA molecules bind to a protein complex, termed RNA-induced silencing complex (RISC), which contains a helicase activity and an endonuclease activity. The helicase activity unwinds the two strands of RNA molecules, allowing the antisense strand to bind to the targeted claudin-1 and/or -23 RNA molecule. The endonuclease activity hydrolyzes the claudin-1 RNA at the site where the antisense strand is bound. Therefore, RNAi is an antisense mechanism of action, as a single stranded (ssRNA) RNA molecule binds to the target claudin-1 and/or -23 RNA molecule and recruits a ribonuclease that degrades the claudin-1 and/or -23 RNA.

An “RNAi-inducing agent” or “RNAi molecule” is used in the invention and includes for example, siRNA, miRNA or shRNA targeted to a claudin-1 and/or -23 transcript or an RNAi-inducing vector whose presence within a cell results in production of an siRNA or shRNA targeted to a target transcript. Such siRNA or shRNA comprises a portion of RNA that is complementary to a region of the target claudin-1 and/or -23 transcript. Essentially, the “RNAi-inducing agent” or “RNAi molecule” downregulates expression of the targeted claudin-1 and/or -23 protein via RNA interference.

Preferably, siRNA, miRNA or shRNA targeting claudin-1 and/or -23 proteins are used.

Exemplary RNAi specific for claudin-23 is available from Santa Cruz Biotechnology (products sc-77716 and sc-77716-SH), as well as Applied Biosystems (products s44021, s-44022, s-44023, 128551, 128552, 290262, and 284899), which are hereby incorporated by reference in their entireties.

Exemplary RNAi specific for claudin-1 are listed below:

(SEQ ID NO: 1)
CLDN1(1) target sequence:
GCAAAGCACCGGGCAGAUA
(SEQ ID NO: 2)
Sense sequence:
AUAGACGGGCCACGAAACGUU
(SEQ ID NO: 3)
Anti-sense strand:
CGUUUCGUGGCCCGUCUAUUU
(SEQ ID NO: 4)
CLDN1(2) target sequence:
GAACAGUACUUUGCAGGCA
(SEQ ID NO: 5)
Sense strand:
ACGGACGUUUCAUGACAAGUU
(SEQ ID NO: 6)
Anti-sense strand:
CUUGUCAUGAAACGUCCGUUU
(SEQ ID NO: 7)
CLDN1(3) target sequence:
UUUCAGGUCUGGCGACAUU
(SEQ ID NO: 8)
Sense sequence:
UUACAGCGGUCUGGACUUUUU
(SEQ ID NO: 9)
Anti-sense strand:
AAAGUCCAGACCGCUGUAAUU

Another exemplary RNAi product specific for claudin-1 includes the mixture of the following dsRNA (A+B+C):

Sense Strand(A):
(SEQ ID NO: 10)
UACAUAGGCAUAGUUCAUGtt
(SEQ ID NO: 11)
CAUGAACUAUGCCUAUGUAtt
Sense Strand(B):
(SEQ ID NO: 12)
AACGUAUGCAGUUAAUUCCtt
(SEQ ID NO: 13)
GGAAUUAACUGCAUACGUUtt
Sense Strand(C):
(SEQ ID NO: 14)
UGAAGAUCUAUGUAUGUGGtt
(SEQ ID NO: 15)
CCACAUACAUAGAUCUUCAtt

Other agents that can be used to interrupt claudin-1 and/or -23 activity include soluble fragments of claudin-1 and/or -23 that consist essentially of one or more extracellular domains of claudin-1 and/or -23, which when delivered to keratinocytes (subsequent to TJ disruption within a region or prior to TJ formation in a particular region) can inhibit claudin-1 and/or -23 dimerization and thereby reduce the efficacy of TJ formation. By way of example, a 27-amino acid peptide corresponding to a portion of the first EL domain (Cldn-153-80) has been shown reversibly to interfered with epithelial barrier function by inducing the rearrangement of key TJ proteins: occludin, claudin-1, junctional adhesion molecule-A, and zonula occludens-1 (Mrsny et al. “A Key Claudin Extracellular Loop Domain Is Critical for Epithelial Barrier Integrity,” Am. J. Pathol. 172(4):905-915 (2008), which is hereby incorporated by reference in its entirety). A soluble (human) CLDN-1 peptide comprises the consensus amino acid sequence of SEQ ID NO: 34 as follows:

SCVSQSTGQ[I/V]QCKVFDSLLNLSSTLQAT

By way of an additional example, a soluble fragment of the first extracellular loop of claudin-23. (Genbank Accession NP 919260, which is hereby incorporated by reference in its entirety) may be used. In one embodiment, a soluble (human) CLDN-23 peptide fragment is derived from the amino acid sequence of SEQ ID NO: 35 as follows

PGWRLVKGFLNQPVDVELYQGLWDMCREQSSRERECGQTDQWGYFEAQP

Homologs of these sequences can also be identified based on conserved amino acid substitutions as is well known in the art.

Other agents include antibodies or aptamers that target the claudin-1 and/or -23 extracellular domains, particularly those that target extracellular loops such as the first EL domain. Antibodies that bind to this region of Claudin-1 are identified in Fofana et al., “Monoclonal Anti-claudin 1 Antibodies Prevent Hepatitis C Virus Infection of Primary Human Hepatocytes,” Gastroenterology 139(3):953-64 (2010), which is hereby incorporated by reference in its entirety.

Suitable fatty acid agents can be identified by screening for displacement of tight junction proteins, including claudin-1 and/or claudin-23, using the procedures identified by Sugibayashia et al., “Displacement of Tight Junction Proteins from Detergent-resistant Membrane Domains by Treatment with Sodium Caprate,” Eur. J. Pharm. Sci. 36(2-3):246-253 (2009); Kurasawa et al., “Regulation of Tight Junction Permeability by Sodium Caprate in Human Keratinocytes and Reconstructed Epidermis,” Biochem Biophys. Res. Commun. 381(2):171-5 (2009), each of which is hereby incorporated by reference in its entirety. One exemplary fatty acid agent is sodium caprate.

Interleukins that can down-regulate claudin-1 and/or claudin-23 expression include, without limitation, IL-17.

Regardless of the embodiment, agents that enhance claudin-1 and/or claudin-23 expression or function, or agents that decrease claudin-1 and/or claudin-23 expression or function, can be administered via pharmaceutical composition. When administered in the form of a pharmaceutical composition, the composition includes one or more of the above-identified agents that modulate claudin-1 and/or claudin-23 expression as well as a pharmaceutically acceptable carrier. The term “pharmaceutically acceptable carrier” refers to any suitable adjuvants, carriers, excipients, or stabilizers, and can be in solid or liquid form such as, tablets, capsules, powders, solutions, suspensions, or emulsions.

Typically, the composition will contain from about 0.01 to 99 percent, preferably from about 20 to 75 percent of active agent(s), together with the adjuvants, carriers and/or excipients. While individual needs may vary, determination of optimal ranges of effective amounts of each component is within the skill of the art. Typical dosages comprise about 0.01 to about 100 mg/kg·body wt. The preferred dosages comprise about 0.1 to about 100 mg/kg·body wt. The most preferred dosages comprise about 1 to about 100 mg/kg·body wt. Treatment regimen for the administration of the agents can also be determined readily by those with ordinary skill in art. That is, the frequency of administration and size of the dose can be established by routine optimization, preferably while minimizing any side effects.

Liposomal or micelle preparations can also be used to deliver the agents of the present invention.

Liposomes are vesicles comprised of one or more concentrically ordered lipid bilayers which encapsulate an aqueous phase. They are normally not leaky, but can become leaky if a hole or pore occurs in the membrane, if the membrane is dissolved or degrades, or if the membrane temperature is increased to the phase transition temperature. Current methods of drug delivery via liposomes require that the liposome carrier ultimately become permeable and release the encapsulated drug at the target site. This can be accomplished, for example, in a passive manner wherein the liposome bilayer degrades over time through the action of various agents in the body. Every liposome composition will have a characteristic half-life in the circulation or at other sites in the body and, thus, by controlling the half-life of the liposome composition, the rate at which the bilayer degrades can be somewhat regulated.

In contrast to passive drug release, active drug release involves using an agent to induce a permeability change in the liposome vesicle. Liposome membranes can be constructed so that they become destabilized when the environment becomes acidic near the liposome membrane (see, e.g., Proc. Natl. Acad. Sci. USA 84:7851 (1987); Biochemistry 28:908 (1989), each of which is hereby incorporated by reference in its entirety). When liposomes are endocytosed by a target cell, for example, they can be routed to acidic endosomes which will destabilize the liposome and result in drug release.

Alternatively, the liposome membrane can be chemically modified such that an enzyme is placed as a coating on the membrane, which enzyme slowly destabilizes the liposome. Since control of drug release depends on the concentration of enzyme initially placed in the membrane, there is no real effective way to modulate or alter drug release to achieve “on demand” drug delivery. The same problem exists for pH-sensitive liposomes in that as soon as the liposome vesicle comes into contact with a target cell, it will be engulfed and a drop in pH will lead to drug release.

Different types of liposomes can be prepared according to Bangham et al., J. Mol. Biol. 13:238-252 (1965); U.S. Pat. No. 5,653,996 to Hsu et al.; U.S. Pat. No. 5,643,599 to Lee et al.; U.S. Pat. No. 5,885,613 to Holland et al.; U.S. Pat. No. 5,631,237 to Dzau et al.; and U.S. Pat. No. 5,059,421 to Loughrey et al., each of which is hereby incorporated by reference in its entirety.

Like liposomes, micelles have also been used in the art for drug delivery. A number of different micelle formulations have been described in the literature for use in delivery proteins or polypeptides, and others have been described which are suitable for delivery of nucleic acids. Any suitable micelle formulations can be adapted for delivery of the therapeutic protein or polypeptide or nucleic acids of the present invention. Exemplary micelles include without limitation those described, e.g., in U.S. Pat. No. 6,210,717 to Choi et al.; and U.S. Pat. No. 6,835,718 to Kosak, each of which is hereby incorporated by reference in its entirety.

When it is desirable to achieve heterologous expression of a protein that promotes claudin-1 and/or claudin-23 expression or RNAi, which knocks down claudin-1 and/or claudin-23 expression, then DNA molecules encoding these products can be delivered into the cell. Basically, this includes providing a nucleic acid molecule encoding the desired product, and then introducing the nucleic acid molecule into the cell under conditions effective to express the desired product in the cell. Preferably, this is achieved by inserting the nucleic acid molecule into an expression vector before it is introduced into the cell.

Any suitable viral or infective transformation vector can be used. Exemplary viral vectors include, without limitation, adenovirus, adeno-associated virus, and retroviral vectors (including lentiviral vectors).

Adenovirus gene delivery vehicles can be readily prepared and utilized given the disclosure provided in Berkner, Biotechniques 6:616-627 (1988) and Rosenfeld et al., Science 252:431-434 (1991), WO 93/07283, WO 93/06223, and WO 93/07282, each of which is hereby incorporated by reference in its entirety. Additional types of adenovirus vectors are described in U.S. Pat. No. 6,057,155 to Wickham et al.; U.S. Pat. No. 6,033,908 to Bout et al.; U.S. Pat. No. 6,001,557 to Wilson et al.; U.S. Pat. No. 5,994,132 to Chamberlain et al.; U.S. Pat. No. 5,981,225 to Kochanek et al.; U.S. Pat. No. 5,885,808 to Spooner et al.; and U.S. Pat. No. 5,871,727 to Curiel, each of which is hereby incorporated by reference in its entirety.

Adeno-associated viral gene delivery vehicles can be constructed and used to deliver into cells a recombinant gene encoding a desired nucleic acid. The use of adeno-associated viral gene delivery vehicles in vitro is described in Chatterjee et al., Science 258:1485-1488 (1992); Walsh et al., Proc. Nat'l Acad. Sci. USA 89:7257-7261 (1992); Walsh et al., J. Clin. Invest. 94:1440-1448 (1994); Flotte et al., J. Biol. Chem. 268:3781-3790 (1993); Ponnazhagan et al., J. Exp. Med. 179:733-738 (1994); Miller et al., Proc. Nat'l Acad. Sci. USA 91:10183-10187 (1994); Einerhand et al., Gene Ther. 2:336-343 (1995); Luo et al., Exp. Hematol. 23:1261-1267 (1995); and Zhou et al., Gene Ther. 3:223-229 (1996), each of which is hereby incorporated by reference in its entirety. In vivo use of these vehicles is described in Flotte et al., Proc. Nat'l Acad. Sci. USA 90:10613-10617 (1993); and Kaplitt et al., Nature Genet. 8:148-153 (1994), each of which is hereby incorporated by reference in its entirety.

Retroviral vectors which have been modified to form infective transformation systems can also be used to deliver a recombinant gene encoding a desired nucleic acid product into a target cell. One such type of retroviral vector is disclosed in U.S. Pat. No. 5,849,586 to Kriegler et al., which is hereby incorporated by reference in its entirety. Lentivirus vectors can also be utilized, including those described in U.S. Pat. No. 6,790,657 to Arya, and U.S. Patent Application Nos. 20040170962 to Kafri et al. and 20040147026 to Arya, each of which is hereby incorporated by reference in its entirety.

Regardless of the type of infective transformation system employed, it should be targeted for delivery of the nucleic acid to a specific cell type. For example, for delivery of the nucleic acid into a cluster of cells, a high titer of the infective transformation system can be introduced directly within the site of those cells so as to enhance the likelihood of cell infection. The infected cells will then express the desired product, in this case RNAi that knocks down expression of claudin-1 and/or claudin-23 or a protein that enhances claudin-1 and/or claudin-23 expression. Alternatively, these infective transformation systems can be administered in combination with a liposomal or micelle preparation, as well as a depot injection.

Ideally, the method involves the local hydrodynamic delivery of the RNAi inducing agent, such as siRNA, miRNA or shRNA etc, to the subject. Although, non-hydrodynamic systemic delivery methods may also be used.

Other delivery methods suitable for the delivery of the RNAi inducing agent (including siRNA, shRNA and miRNA, etc) may also be used. For example, some delivery agents for the RNAi-inducing agents are selected from the following non-limiting group of cationic polymers, modified cationic polymers, peptide molecular transporters, lipids, liposomes and/or non-cationic polymers. Viral vector delivery systems may also be used. For example, an alternative delivery route includes the direct delivery of RNAi inducing agents (including siRNA, shRNA and miRNA) and even anti-sense RNA (asRNA) in gene constructs followed by the transformation of cells with the resulting recombinant DNA molecules. This results in the transcription of the gene constructs encoding the RNAi inducing agent, such as siRNA, shRNA and miRNA, or even as RNA and provides for the transient and stable expression of the RNAi inducing agent in cells and organisms. For example, such an alternative delivery route may involve the use of a lentiviral vector comprising a nucleotide sequence encoding a siRNA (or shRNA) which targets the tight junction proteins. Such a lentiviral vector may be comprised within a viral particle. Adeno-associated viruses (“AAV”) may also be used.

The present invention also includes pharmaceutical or dermatological compositions, which include any of the classes of agents described herein along with an acceptable carrier. The carrier is preferably in the form of a lotion, cream, gel, emulsion, ointment, solution, suspension, foam, or paste. The compositions can be applied to a region of skin by spraying or misting a solution or suspension onto the region of skin, or spreading the lotion, cream, gel, emulsion, ointment, foam or paste onto the region of skin. These compositions may also include, e.g., spermicidal agents such as nonoxynol-9, and can be applied externally as well as intravaginally, as needed.

Alternatively, the pharmaceutical composition can be a vaccine, preferably a transdermal vaccine formulation that would benefit from TJ disruption at the site of vaccine delivery. The transdermal vaccine is often presented in the form of a patch worn by the user, whereby moisture from the vaccine recipient's body allows for delivery of the active agents across the skin (i.e., at the site of application).

Thus, the transdermal vaccine formulations of the present invention preferably include a pharmaceutically suitable carrier, an effective amount of an antigen or antigen-encoding nucleic acid molecule present in the carrier, optionally one or more adjuvants, and an agent that transiently disrupts claudin-1 function within tight junctions. The formulation is presented in the transdermal delivery vehicle, as is known in the art.

Any suitable antigen or antigen-encoding nucleic acid molecule, or a combination thereof, can be used in the vaccine formulations of the present invention. Exemplary classes of vaccine antigen include, without limitation, an allergen, an immunogenic subunit derived from a pathogen, a virus-like particle, an attenuated virus particle, or glycoprotein or glycolipid conjugated to an immunogenic polypeptide. Antigen-encoding nucleic acid molecules can be in the form of naked DNA or expression vectors, as well as infective transformation vectors.

A number of known transdermal vaccine formulations can be modified to include an agent that transiently disrupts claudin-1 and/or claudin-23 expression or function within tight junctions.

One exemplary transdermal vaccine formulation that can be modified is described in U.S. Pat. No. 6,420,176 to Lisziewicz et al., which is hereby incorporated by reference in its entirety. For example, the carrier may comprise one or more of sugar, polylysine, polyethylenimine, polyethylenimine derivatives, and liposomes, together with their derivatives. One preferred carrier of this type is a mannosylated polyethylenimine. The DermaVir transdermal delivery system is believed to employ these types of carriers.

Another exemplary transdermal vaccine formulation that can be modified is described in U.S. Pat. No. 6,869,607 to Buschle et al., which is hereby incorporated by reference in its entirety. For example, the carrier may comprise a solution or emulsion that is substantially free of inorganic salt ions and includes one or more water soluble or water-emulsifiable substances capable of making the vaccine isotonic or hypotonic (e.g., maltose, fructose, galactose, saccharose, sugar alcohol, lipid; or combinations thereof), and an adjuvant that is a polycation (e.g., polylysine or polyarginine) optionally modified with a sugar group. The adjuvant, according to one embodiment, can be a combination of a polycation and an immunostimulatory CpG or non-CpG oligodeoxynucleotide. One form of this adjuvant is the Intercell adjuvant IC31.

Yet another exemplary vaccine formulation that can be modified is described in U.S. Pat. No. 7,247,433 to Rose, which is hereby incorporated by reference in its entirety. For example, HPV virus-like particles could be administered with a pharmaceutically acceptable carrier and with or without E. coli LT R192G as the adjuvant.

The region of skin to be treated in accordance with the present invention is dependent on the intended purpose for delivery. For the vaccine delivery, it is intended that the vaccine be administered to a region of skin such as the upper arm, back, or the like.

Another aspect of the present invention is a transdermal drug formulation. The drug formulation includes a pharmaceutically acceptable carrier, an effective amount of a therapeutic agent, and an agent that transiently disrupts claudin-1 and/or claudin-23 function within tight junctions.

The drug is present in a transdermal delivery device of the invention in a therapeutically effective amount, i.e., an amount effective to bring about a desired therapeutic result in the treatment of a condition. The amount that constitutes a therapeutically effective amount varies according to the particular drug incorporated in the device, the condition being treated, any drugs being coadministered with the selected drug, desired duration of treatment, the surface area of the skin over which the device is to be placed, and other components of the transdermal delivery device. Accordingly it is not practical to enumerate particular preferred amounts but such can be readily determined by those skilled in the art with due consideration of these factors. Generally, however, a drug is present in a transdermal device of the invention in an amount of about 0.01 to about 30 percent by weight based on the total weight of the drug storage material. In a preferred embodiment the drug is substantially fully dissolved, and the drug storage material is substantially free of solid undissolved drug.

The term “drug” and “therapeutic agent” are used interchangeably and are intended to have their broadest interpretation as any therapeutically active substance which is delivered to a living organism to produce a desired, usually beneficial, effect. In general, this includes therapeutic agents in all of the major therapeutic areas including, but not limited to, antiinfectives, antibiotics, antiviral agents, analgesics, fentanyl, sufentanil, buprenorphine, analgesic combinations, anesthetics, anorexics, antiarthritics, antiasthmatic agents, terbutaline, anticonvulsants, antidepressants, antidiabetic agents, antidiarrheals, antihistamines, antiinflammatory agents, antimigraine preparations, antimotion sickness, scopolamine, ondansetron, antinauseants, antineoplastics, antiparkinsonism drugs, cardiostimulants, dobutamine, antipruritics, antipsychotics, antipyretics, antispasmodics, gastrointestinal and urinary, anticholinergics, sympathomimetics, xanthine derivatives, cardiovascular preparations, calcium channel blockers, nifedipine, beta-blockers, beta-agonists, salbutamol, ritodrine, antiarrythmics, antihypertensives, atenolol, ACE inhibitors, diuretics, vasodilators, coronary, peripheral and cerebral, central nervous system stimulants, cough and cold preparations, decongestants, diagnostics, hormones, parathyroid hormone, growth hormone, insulin, hypnotics, immunosuppressives, muscle relaxants, parasympatholytics, parasympathomimetics, anti-oxidants, nicotine, prostaglandins, psychostimulants, sedatives, tranquilizers, skin acting anti-oxidants, caretenoids, ascorbic acid (vitamin C), vitamin E, anti wrinkling agents, retinoids, retinol (vitamin A alcohol), alpha-hydroxic acids, beta-hydroxy acid, salicylic acid, combination-hydroxy acids and poly-hydroxy acids, and hydrolyzed and soluble collagen, moisturizers, hyaluronic acid, anticellulite agents, aminophyllines, skin bleaching agents, retinoic acid, hydroquinone, peroxides, botanical preparations, extracts of aloe-vera, wild yam, hamamelitanin, ginseng, witch hazel, water, green tea, and combinations thereof.

The invention is also useful in the controlled delivery of polypeptide and protein drugs and other macromolecular drugs. These macromolecular substances typically have a molecular weight of at least about 300 daltons, and more typically a molecular weight in the range of about 300 to 40,000 daltons. In one embodiment, the therapeutic is at least 300 daltons in size. In another embodiment, the therapeutic is at least 500 daltons in size. In yet a further embodiment, the therapeutic is not less than 300 daltons in size.

Specific examples of peptides, and proteins and macromolecules in this size range include, without limitation, LHRH, LHRH analogs such as buserelin, gonadorelin, napharelin and leuprolide, GHRH, GHRF, insulin, insulotropin, heparin, calcitonin, octreotide, endorphin, TRH, NT-36 (chemical name: N=[[(s)-4-oxo-2-azetidinyl]carbonyl]-L-histidyl-L-prolinamide), liprecin, pituitary hormones (e.g., HGH, HMG, HCG, desmopressin acetate, etc.), follicle luteoids, αANF, growth factors such as growth factor releasing factor (GFRF), βMSH, somatostatin, atrial natriuretic peptide, bradykinin, somatotropin, platelet-derived growth factor, asparaginase, bleomycin sulfate, chymopapain, cholecystokinin, chorionic gonadotropin, corticotropin (ACTH), epidermal growth factor, erythropoietin, epoprostenol (platelet aggregation inhibitor), follicle stimulating hormone, glucagon, hirulog, and other analogs of hirudin, hyaluronidase, interferon, insulin-like growth factors, interleukin-1, interleukin-2, menotropins (urofollitropin (FSH) and LH), oxytocin, streptokinase, tissue plasminogen activator, urokinase, vasopressin, desmopressin, ACTH analogs, ANP, ANP clearance inhibitors, angiotensin II antagonists, antidiuretic hormone agonists, antidiuretic hormone antagonists, bradykinin antagonists, CD4, ceredase, CSF's, enkephalins, FAB fragments, IgE peptide suppressors, IGF-1, neuropeptide Y, neurotrophic factors, oligodeoxynucleotides and their analogues such as antisense RNA, antisense DNA and anti-gene nucleic acids, opiate peptides, colony stimulating factors, parathyroid hormone and agonists, parathyroid hormone antagonists, prostaglandin antagonists, pentigetide, protein C, protein S, ramoplanin, renin inhibitors, thymosin alpha-1, thrombolytics, TNF, vaccines, vasopressin antagonist analogs, alpha-1 anti-trypsin (recombinant), and TGF-beta.

Yet another aspect of the present invention relates to a transdermal vaccine or drug delivery device or patch. The transdermal drug delivery device comprises a transdermal vaccine or drug formulation according to the present invention. In one embodiment, the transdermal vaccine or drug delivery patch includes a backing material, an adhesive material in contact with a first portion of the backing material; and a drug storage material comprising the transdermal vaccine or drug formulation, where the drug storage material is in contact with a second portion of the backing material. In one embodiment the patch also includes a releasable liner material to be removed upon application to the skin.

Any suitable backing material known in the art of transdermal patches (such as a breathable material) may be used in accordance with the present invention. The backing is flexible such that the device conforms to the skin. Exemplary backing materials include conventional flexible backing materials used for pressure sensitive tapes, such as polyethylene, particularly low density polyethylene, linear low density polyethylene, high density polyethylene, polyester, polyethylene terephthalate, randomly oriented nylon fibers, polypropylene, ethylene-vinyl acetate copolymer, polyurethane, rayon and the like. Backings that are layered, such as polyethylene-aluminum-polyethylene composites, are also suitable. The backing should be substantially inert to the ingredients of the drug storage material.

Adhesives suitable for use with the present invention will any dermatologically acceptable adhesive. Examples of dermatologically acceptable adhesives include, but are not limited to acrylics, natural and synthetic rubbers, ethylene vinyl acetate, poly(alpha-olefins), vinyl ethers, silicones, copolymers thereof and mixtures thereof. In an embodiment, the first adhesive layer includes a silicone adhesive (e.g., BIO-PSA 7-4302 Silicone Adhesive available commercially from Dow Corning®).

The transdermal patch may optionally include one or more release liners for storage or handling purposes. Many suitable release liners are known within the art. The release liner can be made of a polymeric material that may be optionally metallized. Examples of suitable polymeric materials include, but are not limited to, polyurethane, polyvinyl acetate, polyvinylidene chloride, polypropylene, polycarbonate, polystyrene, polyethylene, polyethylene terephthalate (PET), polybutylene terephthalate, paper, and combinations thereof. In certain embodiments, the release liner is siliconized. In other embodiments, the release liner is coated with fluoropolymer, such as PET coated with fluoropolymer (e.g., SCOTCHPAK™ 9744 from 3M™).

The drug storage material may be any dermatologically acceptable material suitable for use as a drug storage material or reservoir in a transdermal patch. For instance, the drug storage material may be a polymer. Examples of polymers include microporous polyolefin film (e.g., SOLUPOR® from SOLUTECHT™), acrylonitrile films, polyethylnapthalene, polyethylene terephthalate (PET), polyimide, polyurethane, polyethylene, polypropylene, ethylene-vinyl acetate (EVA), copolymers thereof and mixtures thereof. In one embodiment, the polymer is EVA. In another embodiment, the polymer is EVA having a vinyl acetate content by weight in the range of about 4% to about 19%. In a preferred embodiment, the polymer is EVA having vinyl acetate content by weight of about 9%. The drug storage material may also include a heat-sealable material for attaching to other components. As an example, the heat-sealable permeable layer may be an EVA membrane, such as COTRAN™ 9702, available commercially from 3M™.

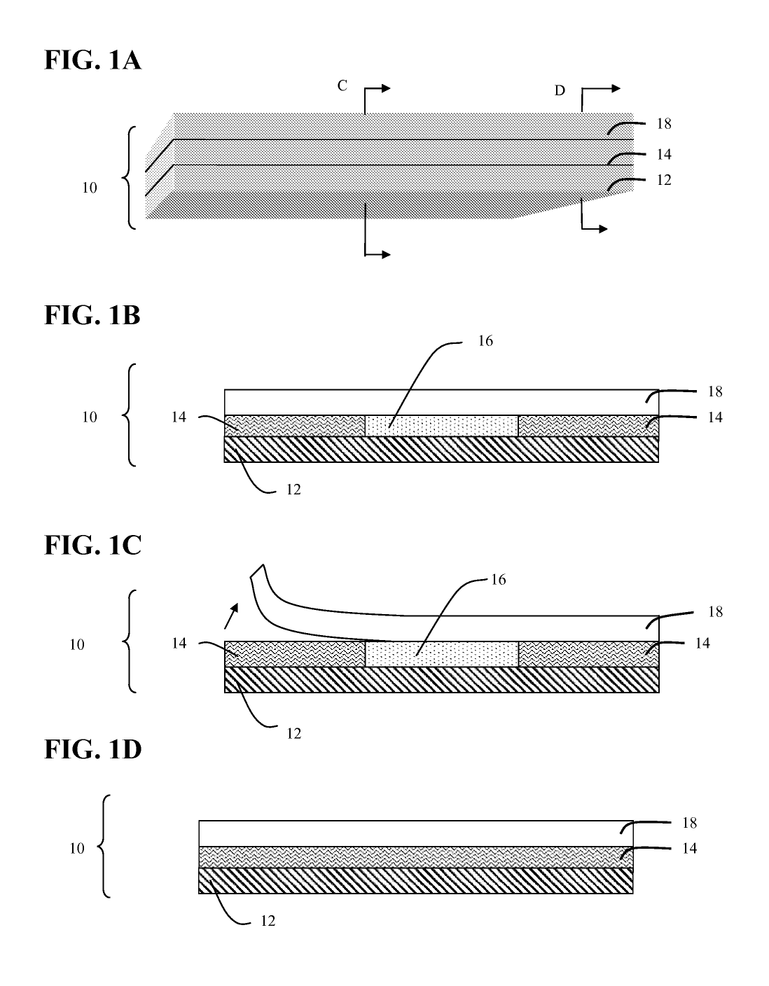

Referring now to FIGS. 1A to 1D, FIG. 1A is a perspective view of one embodiment of a transdermal patch according to the present invention. FIG. 1B is a cross-section of transdermal patch 10 along axis C of FIG. 1A. In one embodiment, transdermal patch 10 includes backing 12, adhesive material 14, and drug storage material 16. In addition, transdermal patch 10 may optionally include releasable liner 18, which is removed upon application to skin, as shown in FIG. 1C. FIG. 1D is a cross-sectional view of transdermal patch 10 along axis D of FIG. 1A.

### EXAMPLES

The following examples are provided to illustrate embodiments of the present invention but are by no means intended to limit its scope.

### Materials and Methods for Examples 1 to 7

### Study Participants—Expression Profiling and Validation Experiments

Clinical characteristics of AD patients and NA controls enrolled as part of the NIAID-funded ADVN are shown in Table 3 and reviewed in a previous publication. Beck et al., “Phenotype of Atopic Dermatitis Subjects With a History of Eczema Herpeticum,” J. Allergy Clin. Immunol. 124:260-9, 9 e1-7 (2009), which is hereby incorporated by reference in its entirety. The diagnosis of AD was made using the U.S. consensus conference criteria. Eichenfield, L. F., “Consensus Guidelines in Diagnosis and Treatment of Atopic Dermatitis,” Allergy 59 Suppl 78:86-92 (2004), which is hereby incorporated by reference in its entirety. All AD subjects had extrinsic disease as defined by serum total IgE≧2 SD of age-dependent norms and a positive multi-allergen RAST (ImmunoCap Phadiatop™). AD severity was defined according to the ‘eczema area and severity index’ (“EASI”), a standardized grading system. Hanifin et al., “The Eczema Area and Severity Index (EASI): Assessment of Reliability in Atopic Dermatitis. EASI Evaluator Group,” Exp. Dermatol. 10:11-8 (2001), which is hereby incorporated by reference in its entirety. Non-atopic, healthy subjects were defined as having no personal or family history of atopic diseases, no personal history of chronic skin or systemic diseases and a serum total IgE that was 2 SD of age-dependent norms and a negative Phadiatop™. The diagnosis of psoriasis (“PS”) was based on characteristic clinical features of plaque-type lesions by a board-certified dermatologist. Exclusion criteria were as follows: age <18 yrs, >60 yrs, use of systemic immunosuppressive therapy, leukotriene inhibitors within the last six weeks, use of topical steroids or calcineurin inhibitors within the last six weeks at the site of blister formation or biopsy, and subjects with a recent systemic infection or course of oral antibiotics within last two weeks. All subjects either underwent an epidermal procurement procedure (see below) or a 4 mm punch biopsy of their non-sunexposed forearm. There was no statistically significant difference between African Americans (“AA”) and European Americans (“EA”) in these severity scorings after adjusting for age and gender.

### Epidermal Procurement and Processing

Epidermal samples were obtained from five extrinsic AD subjects (2 male, 3 female; mean age 34±6 yrs), five healthy nonatopic control subjects (4 male, 1 female; mean age 28±8 yrs) and four psoriasis (“PS”) subjects (2 male, 2 female; mean age 30±8 yrs). For validation studies, eleven extrinsic AD subjects (1 male, 10 female; mean age 31±3 yrs) and twelve NA control subjects (7 male, 5 female; mean age 31±3 yrs) underwent skin biopsies from nonlesional sites on the non-sunexposed, volar forearm. The findings were also validated in additional epidermal samples obtained from six extrinsic AD (2 male, 4 female; mean age 34±6 yrs) and six NA subjects (2 male, 4 female; mean age 32±2 yrs).

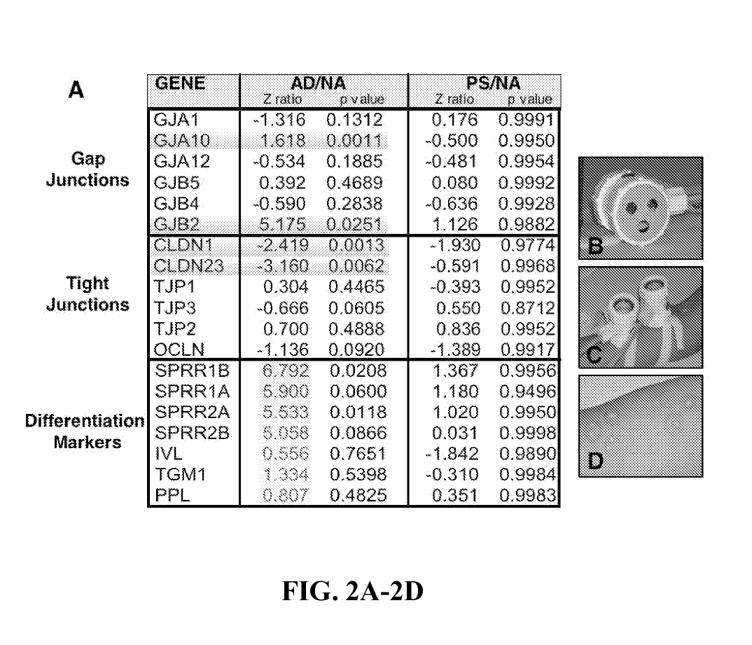

An NP-2 negative pressure vacuum apparatus (FIG. 2B) (Electronic Diversities, Finksburg, Md., USA) was applied to the volar forearm (FIG. 2C). The blister roof, which consists of full thickness epidermis (FIG. 2D), was removed using sterile technique and placed in Hank's Balanced Salt Solution (Invitrogen). Total RNA was extracted from epidermis using the QlAshredder spin column (Qiagen) and RNeasy RNA isolation kits (Qiagen). The quality of total RNA samples (RNA Integrity Number, RIN) was assessed using an Agilent 2100 Bioanalyzer (Agilent Technologies, Palo Alto, Calif.). Samples were selected for microarray analysis if they had a good RIN (range 8-10).

### Gene Expression Profiling

Biotin-labeled, complementary RNA (cRNA) was prepared from total RNA according to manufacturer's protocol (Illumina, San Diego, Calif.). cRNA was hybridized to Illumina Sentrix HumanRef-8 Expression BeadChips (Illumina, San Diego, Calif.), which contain 24,350 probes corresponding to 21,429 unique genes. Signal intensity quantification was performed using an Illumina BeadStation 500GX Genetic Analysis Systems scanner.

Preliminary analysis of the scanned data was performed using Illumina BeadStudio software which returns single intensity data values for each probe following the computation of a trimmed mean average for each probe represented by a variable number of bead probes on the array. Z-transformation for normalization was performed on each Illumina sample/array on a stand-alone basis and Z ratios were calculated by taking the difference between the averages of the observed gene Z scores and dividing by the standard deviation of all the differences for that particular comparison. Cheadle et al., “Application of Z-Score Transformation to Affymetrix Data,” Appl. Bioinformatics 2:209-17 (2003), which is hereby incorporated by reference in its entirety. Significant changes in gene expression between class pairs were calculated by Z test (Nadon and Shoemaker, “Statistical Issues With Microarrays: Processing and Analysis,” Trends Genet. 18:265-71 (2002), which is hereby incorporated by reference in its entirety) and significant genes were defined as those which satisfy a Z test p-value of ±1.5E-3. For FIGS. 7A and 7B, the BeadStudio expression values for each sample/array were scaled to have median 256 and then log₂ transformed.

Quantitative PCR (qPCR)

QPCR was performed using the iScript™ cDNA Synthesis kit and iQ™ SYBER Green Supermix assay system (Bio-Rad Laboratories). All PCR amplifications were carried out in triplicate on an iQ5 Multicolor real-time PCR detection system (Bio-Rad). Primers were designed and synthesized by Integrated

DNA Technologies (Table 1). Relative gene expression was calculated by using the 2^{-ΔΔCt} method. Livak and Schmittgen, “Analysis of Relative Gene Expression Data Using Real-Time Quantitative PCR and the 2(-Delta Delta C(T)) Method,” Methods 25:402-8 (2001), which is hereby incorporated by reference in its entirety. The normalized Ct value of each sample was calculated using GAPDH as an endogenous control gene.

TABLE 1
Real-time PCR Primers

| Target | Forward | Reverse |
|---|---|---|
| GAPDH | GAAGGTGAAGGTCGGAGTC | GAAGATGGTGATGGGATTTC |
| | (SEQ ID NO: 16) | (SEQ ID NO: 17) |
| Cldn-1 | CGATGAGGTGCAGAAGATGA | CCAGTGAAGAGAGCCTGACC |
| | (SEQ ID NO: 18) | (SEQ ID NO: 19) |
| Cldn-23 | CTTGCCATGCAAACTCTCAA | TTCTCCTCTTGGCTTCTGGA |
| | (SEQ ID NO: 20) | (SEQ ID NO: 21) |
| Occludin | CTGGCCTACAGGAATACAAG | CTTGATGTGTGACAATTTGC |
| | (SEQ ID NO: 22) | (SEQ ID NO: 23) |
| Z0-1 | CGGAAAACATGCTACACAC | CCCATTTACTGGCTGGTAT |
| | (SEQ ID NO: 24) | (SEQ ID NO: 25) |
| GJB2 | GTTTAACGCATTGCCCAGTT | GGCCTACAGGGGTTTCAAAT |
| (Connexin-26) | (SEQ ID NO: 26) | (SEQ ID NO: 27) |
| PVRL1 | AGCCATTAAGGAGAAACGA | TTCCCAATTTCTCTGCTCT |
| (Nectin-1) | (SEQ ID NO: 28) | (SEQ ID NO: 29) |
| CDH | CAGAAAGTTTTCCACCAAAG | AAATGTGAGCAATTCTGCTT |
| (E-cadherin) | (SEQ ID NO: 30) | (SEQ ID NO: 31) |
| FLG | GAGCTGAAGGAACTTCTGG | GATCCATGAAGACATCAACCA |
| | (SEQ ID NO: 32) | (SEQ ID NO: 33) |

### Imaging Tight Junction Proteins

For immunohistochemical staining the following Abs were used: claudin-1 (0.2 μg/ml, JAY.8; Zymed), occludin (2.5 μg/ml, OC-3F10; Zymed), ZO-1 (2.5 μg/ml, ZO1-1A12; Zymed), or isotype control. Five μm sections from formalin-fixed skin biopsies were deparaffinized and rehydrated. Slides were incubated in 1×EDTA solution, pH 8.0 at 95° C. for 10 min. Samples were incubated overnight at 4° C. with primary Abs titered to the lowest concentration that produced immunoreactivity in control samples. The secondary antibodies and detection system used were reported previously. Beck et al., “Detection of the Chemokine RANTES and Endothelial Adhesion Molecules in Nasal Polyps,” J. Allergy Clin. Immunol. 98:766-80 (1996), which is hereby incorporated by reference in its entirety. For immunofluorescence labeling, skin samples were incubated in blocking solution (5% BSA, 0.1% saponin, 1 mM calcium in PBS) for 20 min, followed by 90 min incubation with primary antibodies diluted in blocking solution. This was followed by a 60 min incubation with secondary antibodies; 1:1000 AlexaFluor 488 donkey-anti-rabbit IgG H+L (Molecular Probes), 1:1000 Alexa Fluor 568 donkey-anti-mouse IgG H+L (Molecular Probes), 1:10,000 4′,6-diamidino-2-phenyl-indole, dihydrochloride (DAPI) (Molecular Probe). PHK grown on transwell filters or a glass coverglass were fixed in methanol at −20° C. for 15 min, followed by blocking in PBS with 1% BSA and immunolabeled with the above TJ antibodies.

Fluorescent images were obtained with an Olympus FV1000 laser scanning confocal microscope using the FV10-ASW 1.7 software. The Alexa Fluor 488 and 568 signals were imaged sequentially in frame interlace mode to eliminate crosstalk between channels. Saturation level of fluorescence intensity was set for the controls using the Hi-Lo feature of the Fluoview software. For each experiment, the images were captured at identical settings during image acquisition and no manipulations of the images occurred prior to importing into the FV1000 software at the workstation.

Ex vivo Electrophysiological Measurements in Ussing Chambers

Isolated blister roofs (1 cm in diameter; FIG. 2B-D) were placed in the Ussing chamber in 2 mm diameter sliders for Snapwell chambers (area=0.031 cm²; Physiologic Instruments) and bathed in Ringer's lactate and gassed with a mixture of 95% O₂ and 5% CO₂. The tissues were continuously voltage-clamped to zero using VCC MC8 (Physiologic Instruments, San Diego, Calif., USA). Transepithelial short-circuit current (I_{sc}, expressed as μA/cm² tissue surface area) were measured and total tissue conductance (G, expressed as mS/cm² tissue surface area) was calculated using Ohm's law by applying a 5 mV transepithelial pulse every 20 sec and measuring resulting current deflections.

Dilution potential measurements were performed to determine the changes in the permeability ratio between the Na⁺ and Cl⁻ using the Nernst equation. Berry et al., “Ion Selectivity and Proximal Salt Reabsorption,” Am. J. Physiol. 235:F234-45 (1978); Vidyasagar and Ramakrishna, “Effects of Butyrate on Active Sodium and Chloride Transport in Rat and Rabbit Distal Colon,” J. Physiol. 539:163-73 (2002); and Hallani et al., “Characterization of Calcium-Activated Chloride Channels in Patches Excised From the Dendritic Knob of Mammalian Olfactory Receptor Neurons,” J. Membr. Biol. 161:163-71 (1998), which are hereby incorporated by reference in their entirety. Briefly, the tissues were allowed to equilibrate in the Ussing chamber for 45 min. Ten nM bumetanide was added to both the apical/corneum and basolateral/basal bathing solutions to block Na⁺—K⁺-2Cl⁻ cotransport that feeds transepithelial Cl⁻ secretion via apical channels. Dilution potentials were induced by apical and/or basolateral perfusion with Ringer solutions containing two different concentrations of Na⁺ (140 and 70 mM) and Cl⁻ (119.8 and 50 mM) and the remaining replaced with mannitol to maintain equal osmolarity between experiments. Dilution potentials were corrected for changes in junction potential (usually less than 1 mV). Change in membrane voltage (Eₘ) along with known concentrations of Na⁺ and Cl⁻ in the respective solutions were substituted in the modified Nernst equation to determine the change in permeability ratio between Cl⁻ and Na⁺ (P_{Cl}[Cl]ᵢ/P_{Na}[Na]ᵢ). Eₘ=RT/F*2.303 log₁₀{P_{Na}[Na]ₒ+P_{Cl}[Cl]ᵢ/P_{Na}[Na]ᵢ+P_{Cl}[Cl]ₒ} R=8.314472 (J/K/mol); F=96.48531(KJ/mol); Permeability ratio (β)=P_{Cl}/P_{Na}; T=310 (Kelvin).

### Permeability Studies in Ussing Chambers

For the permeability assay 0.02% of FITC-albumin (Sigma) was added to the apical side of the inserts and buffer samples were collected at several time points (0.5-3 h) from the other side of the membrane. Permeability was assessed using a spectrophotometer (Multiskan EX; Thermo Electron Corporation, Finland) at 490 nm. Permeability data were expressed as FITC fluorescence intensity normalized to sample area and time (O.D./cm²/h).

### Culture of Primary Human Foreskin Keratinocytes (PHK)

Human keratinocytes were isolated from neonatal foreskin. Poumay et al., “Basal Detachment of the Epidermis Using Dispase: Tissue Spatial Organization and Fate of Integrin Alpha 6 Beta 4 and Hemidesmosomes,” J. Invest. Dermatol. 102:111-7 (1994), which is hereby incorporated by reference in its entirety. PHK were cultured in Keratinocyte-SFM (Invitrogen/Gibco) with 1% Pen/Strep, 0.2% Amphotericin B (Invitrogen/Gibco). To differentiate PHK, cells were grown in DMEM (Invitrogen/Gibco) with 10% heat-inactivated fetal bovine serum (Invitrogen/Gibco) and 1% Pen/Strep, 0.2% Amphotericin B (Invitrogen/Gibco). For TJ modulation experiments, the following cytokines were added alone or in combination to the differentiation media; human IL-4 (50 ng/ml; R&D system) and IL-13 (50 ng/ml; R&D system).

### Trans Epithelial Electric Resistance (TEER) and Paracellular Flux

PHK were plated at a subconfluent density of 2.5−3×10⁴ cells/filter in Transwell insert and cultured in Keratinocyte-SFM media until confluent. TEER was measured using an EVOMX voltohmmeter (World Precision Instruments, Sarasota, Fla.). The resistance of cell-free filters was subtracted from each experimental value.

To evaluate the paracellular flux of PHK, 0.02% Fluorescein (FITC) Sodium (Fluka) in HBSS was added to the upper chamber. Samples were collected from the lower chamber at different times (0.5-3 h). The amount of FITC that had diffused from the apical to the basal side of the filter was measured using a spectrofluorimeter (Multiskan EX; Thermo Electron Corporation, Finland) with excitation/emission wavelengths 490/514 nm. Paracellular permeability was presented as “Relative Fluorescence Flux”=Experimental condition/Filter alone×100.

### Immunoblotting

PHK were lysed on ice in RIPA lysis buffer (20 mM Tris, 50 mM NaCl, 2 mM EDTA, 2 mM EGTA, 1% sodium deoxycholate, 1% TX-100, and 2% SDS, pH 7.4), with 1:100 Protease Inhibitor Cocktail (Sigma), 1:100 Phosphatase Inhibitor Cocktail (SigmaCells for 30 min at 4° C. The samples were heated at 95° C. for 10 min and then centrifuged at 14,000 rpm for 15 min. Forty μg of protein, determined by BCA (Pierce) in NuPage LDS Sample Buffer (Invitrogen) was applied to 4-12% NuPage Bis-Tris gels (Invitrogen). Electrophoresis was performed under reducing conditions with MES SDS Buffer (Invitrogen). Membranes were incubated in blotting solution (5% non-fat dry milk in PBS+0.05% Tween 20) at RT for 1 h and then incubated with primary antibodies; claudin-1 (JAY.8; Zymed), occludin (OC/3F10; Zymed) and GAPDH (FL-355; Santa Cruz). An HRP-linked secondary antibody (GE Healthcare) was used with ECL (GE Healthcare) to visualize bands by autoradiography with Kodak BioMax MR film. The pixel intensity of each band was estimated with Image J.

### RNA Interference

PHK were plated on glass coverslips at 2-3×10⁵ cells/well in a 6-well plate or at 2-3×10⁴ cells/filter in Transwell insert (Costar; PET membrane, 0.4 μm pore size, 6.5 mm insert) in Keratinocyte-SFM without antibiotics. The next day after plating, cells were transfected with claudin-1 specific or control (scrambled) siRNAs (Santa Cruz) using Lipofectamine™ 2000 transfection Reagent (Invitrogen). TEER and permeability experiments were conducted 48 hours after PHK were switched to differentiation media.

### Study Participants and Phenotypes (ADVN Cohort)

DNA was isolated using standard protocols from 258 unrelated European American AD patients and 156 healthy controls participating in the ADVN. The same set of markers was genotyped on 176 African American AD patients, and 152 healthy controls. Baseline characteristics are presented in Table 3 and further details can be found in previous publications. Beck et al., “Phenotype of Atopic Dermatitis Subjects With a History of Eczema Herpeticum,” J. Allergy Clin. Immunol. 124:260-9, 9 e1-7 (2009), which is hereby incorporated by reference in its entirety. AD and NA controls were diagnosed and as described above.

### Genotyping and Quality Control

Genotyping was performed on genomic DNA extracted from blood samples using MagAttract DNA blood Mini M48 kit (QIAGEN) on a Biorobot M48, according to the manufacturer's instructions. DNA quantification was performed using Pico-Green (Pico-green, Molecular Probes). Genotyping in these samples was determined for each of the selected tagSNPs with the Illumina GoldenGate custom panel containing 384-plex assays according to the manufacturer's protocol (Illumina Inc., San Diego, Calif.).

Tagging SNPs were selected to represent the CLDN1 gene in both the EA and AA groups. The SNP selection approach was to examine 10 kb upstream and 10 kb downstream in accordance with design score validations based on Illumina in-house measurements and the 60-bp limitation (a SNP cannot be closer than 60 by to another SNP on this OPA). All available CLDN1 SNPs were initially selected from the HapMap to tag the linkage disequilibrium (LD) blocks in each of the ethnic groups (EA and AA). Tagging was based on the LDSelect algorithm (Carlson et al., “Selecting a Maximally Informative Set of Single-Nucleotide Polymorphisms for Association Analyses Using Linkage Disequilibrium,” Am. J. Hum. Genet. 74:106-20 (2004); Howie et al., “Efficient Selection of Tagging Single-Nucleotide Polymorphisms in Multiple Populations,” Hum. Genet. 120:58-68 (2006), which are hereby incorporated by reference in their entirety), with a minor allele frequency (MAF) ≧10% and an r² threshold of 0.80 (as reported in HapMap) to ensure nearly perfect linkage disequilibrium (LD) in order to infer information on all SNPs captured by the tag set. A final selection included 27 SNPs chosen for the Illumina OPA. Of the 27 tagging SNPs selected, 24 qualified as tagging SNPs from both the HapMap CEPH Utah (CEU, with European ancestry) and the HapMap Yoruba (YR1, with African ancestry) samples; an additional three tagging SNPs (rs6800425, rs1155884, and rs9809713) were genotyped only in the AAs. Two LD blocks were observed among the European American group (block 1, rs10212165, rs3954259 and rs9290929 (D′=0.982-1.0); block 2, rs9835663 and rs3732923 (D′=0.976), and three LD blocks were observed among the African American group (block 1, rs3954259 and rs9290929 (D′=1.0); block 2, rs893051, rs9839711 and rs9835663 (D′=0.957-1.0); block 3, rs6800425 and rs3774028 (D′=1)) using the criteria of Gabriel et al., “The Structure of Haplotype Blocks in the Human Genome,” Science 296:2225-9 (2002), which is hereby incorporated by reference in its entirety.

The 27 SNPs were genotyped using the custom-designed Illumina oligonucleotide pool assay (OPA) for the BeadXpress Reader System and the GoldenGate Assay with VeraCode Bead technology (San Diego, Calif., USA) according to the manufacturer's protocol. Fan et al., “Illumina Universal Bead Arrays,” Methods Enzymol. 410:57-73 (2006), which is hereby incorporated by reference in its entirety. In particular, genotyping was performed on genomic DNA extracted from blood samples using MagAttract DNA blood Mini M48 kit (QIAGEN) on a Biorobot M48, according to the manufacturer's instructions. DNA quantification was performed using Pico-Green (Pico-green, Molecular Probes). Genotyping in these samples was determined for each of the selected tagSNPs with the Illumina GoldenGate custom panel containing 384-plex assays according to the manufacturer's protocol (Illumina Inc., San Diego, Calif.). Briefly, the GoldenGate assay employs three primers designed for each locus. Two are specific to each allele at the SNP site and a third hybridizes at a downstream locus from the site. All three primers have regions complementary to both genome and universal PCR primer sites. A total of 250 ng of high quality gDNA was plated and then activated. The activated DNA, paramagnetic particles, assay oligos, and hybridization buffer are combined in a hybridization step to allow DNA to bind to the particles. Following hybridization of primers, plates were washed to reduce noise and allele specific oligos were extended and ligated to the downstream locus specific primer. This mix then served as a PCR template using the universal primers, P1, P2, and P3. P1 and P2 are Cy3 and Cy5 labeled. After down-stream processing, the single-stranded dye-labeled PCR products were hybridized to their complement VeraCode bead type. Plates were then scanned in the BeadXpress Reader for fluorescence and code identification. Scanned data and oligo assignments were uploaded into Illumina's BeadStudio software for downstream genotype cluster analysis. Genotyping quality was high with an average completion rate of 97.2-98.2% for the BeadXpress genotyping.

All samples were also genotyped for the two FLG mutations most commonly associated with AD in EA (R501X and 2282de14), plus 9 additional polymorphisms (rs12730241, rs2065956, rs11582620, rs3126082, rs6587665, rs11204980, rs1933063, rs1933064, rs3126091) as described. Gao et al., “Filaggrin Mutations That Confer Risk of Atopic Dermatitis Confer Greater Risk for Eczema Herpeticum,” J. Allergy Clin. Immunol. 124:507-13, 13 e1-7 (2009), which is hereby incorporated by reference in its entirety. Interaction among FLG mutations previously associated with AD as well as haplotype-tagging SNPs in FLG and CLDN1 SNPs were investigated using PLINK epistasis. DNA samples from subjects enrolled for the immunostaining study were also genotyped for the same 2 FLG null mutations.

### Statistical Analysis

Data were expressed as mean±SEM of 3 or more experiments. Differences between groups were evaluated using an appropriate t-test. A P value of ≦0.05 was considered statistically significant. The statistical analysis performed as part of the expression profiling or SNP analyses are described in that section. The Cochran-Armitage trend test was used to test for association between each individual marker (under an additive model) and disease status using PLINK software. Analyses were performed for subjects of European and African ancestry separately to minimize confounding due to racial differences in polymorphism frequency. Association between genetic markers and the quantitative measure of severity, EASI, was tested for using recessive logistic regression models. Departures from Hardy-Weinberg equilibrium (HWE) at each locus were tested by means of the chi-squared test separately for cases and controls using PLINK; all SNPs were in HWE. Tests for association that had a P value <0.05 were then confirmed with PLINK max(T) test using 10,000 permutations.

### Example 1

### Claudin-1 Expression is Markedly Reduced in Nonlesional AD Epidermis

To characterize and quantify the expression of human epidermal proteins important for barrier function, gene expression profiling of nonlesional or clinically unaffected epidermis was performed using blister roofs from AD, psoriasis (“PS”), and NA subjects (FIG. 2B-D). The Illumina Sentrix HumanRef-8 Chip contained 43 TJ genes (see Table 2), 8 gap junction genes, and 41 epidermal differentiation complex (“EDC”) genes. Table 2 shows results from modulation of tight junction pathway genes (n=43) in epidermal samples taken from atopic dermatitis compared to healthy NA controls.

TABLE 2
Modulation of Tight Junction Pathway Genes

| | | | Fold |
|---|---|---|---|
| Gene | Z ratio | P value | Change |
| ACTN1 | 2.796 | 0.0121 | 1.47 |
| ACTN4 | 0.798 | 0.3742 | 1.14 |
| CGN | 0.063 | 0.9532 | 1.04 |
| CIP98 | 0.712 | 0.1986 | 1.1 |
| CLDN1 | −2.419 | 0.0013 | −1.42 |
| CLDN10 | 1.620 | 0.0641 | 1.27 |
| CLDN12 | −0.798 | 0.2518 | −1.09 |
| CLDN14 | −1.056 | 0.0896 | −1.13 |
| CLDN15 | 0.553 | 0.4967 | 1.12 |
| CLDN16 | −0.007 | 0.9881 | −1.02 |
| CLDN18 | −0.915 | 0.2421 | −1.08 |
| CLDN23 | −3.155 | 0.0062 | −1.6 |
| CLDN4 | −0.977 | 0.4279 | −1.07 |
| CLDN6 | −1.067 | 0.1561 | −1.02 |
| CLDN7 | −0.298 | 0.6320 | −1.03 |
| CLDN8 | −1.265 | 0.1616 | −1.26 |
| CRB3 | 0.755 | 0.3508 | 1.14 |
| CSDA | −0.426 | 0.4062 | −1.02 |
| CTNNA1 | 0.139 | 0.8766 | 1.02 |
| CTNNB1 | −0.879 | 0.1319 | −1.1 |
| INADL | 0.627 | 0.3357 | 1.12 |
| LLGL2 | −0.455 | 0.4178 | −1.06 |
| MAGI1 | 0.694 | 0.2086 | 1.12 |
| MLLT4 | 0.374 | 0.4000 | 1.04 |
| MPDZ | 0.616 | 0.1559 | 1.06 |
| MPP5 | −0.136 | 0.8746 | 1.11 |
| NR3C1 | −1.299 | 0.0178 | −1.16 |
| OCLN | −1.136 | 0.0920 | −1.17 |
| PARD3 | −0.621 | 0.3375 | −1.06 |
| PPP2CA | 0.170 | 0.7934 | 1.03 |
| PRKCA | 0.523 | 0.3959 | 1.02 |
| PRKCI | 0.329 | 0.6671 | 1.04 |
| RAB13 | 0.532 | 0.4387 | 1.13 |
| SEC6L1 | 0.959 | 0.2246 | 1.19 |
| SEC8L1 | 0.494 | 0.5482 | 1.07 |
| SPTAN1 | −1.965 | 0.0017 | −1.21 |
| SRC | −0.656 | 0.1983 | −1.05 |
| TJP1 | 0.304 | 0.4465 | 1.04 |
| TJP2 | 0.700 | 0.4888 | 1.14 |
| TJP3 | −0.666 | 0.0605 | −1.07 |
| TJP4 | 0.081 | 0.8330 | −1.02 |
| YES1 | 0.734 | 0.0692 | 1.08 |
| ZAK | −0.287 | 0.6614 | 1.05 |

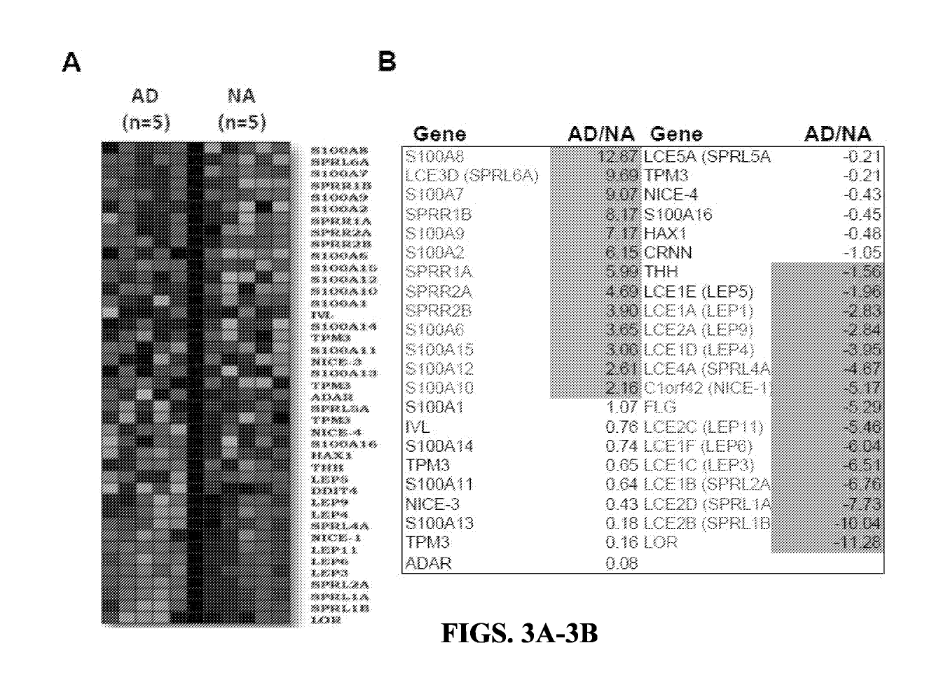

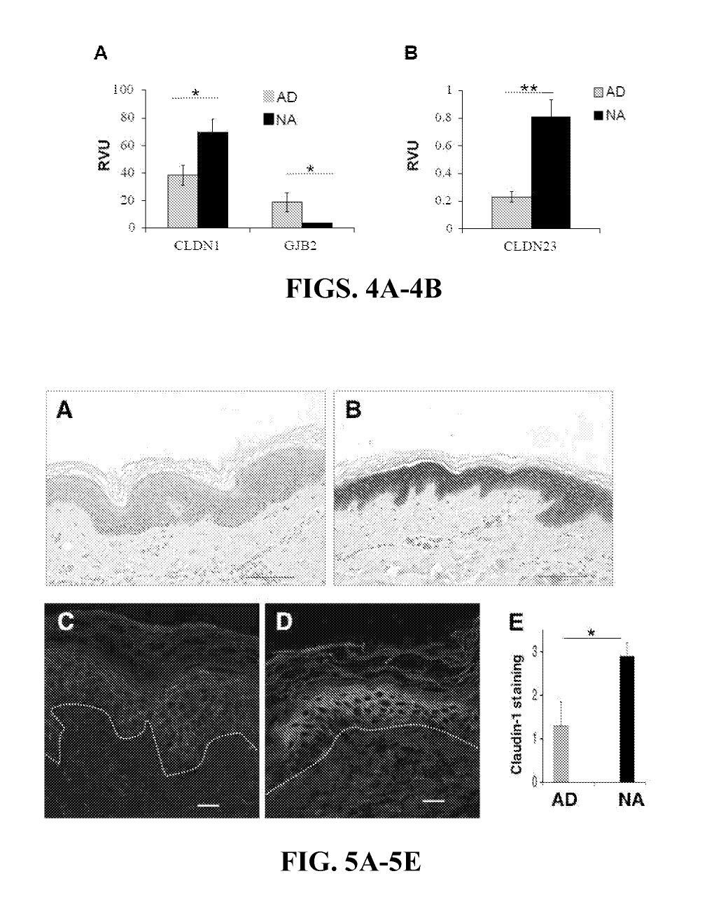

A number of the EDC genes were differentially expressed in AD versus NA nonlesional epidermis and several (e.g., S100A8, S100A7, FLG and LOR) (see FIGS. 3A-B) corroborate the findings observed by others in lesional skin biopsies. Sugiura et al., “Large-Scale DNA Microarray Analysis of Atopic Skin Lesions Shows Overexpression of an Epidermal Differentiation Gene Cluster in the Alternative Pathway and Lack of Protective Gene Expression in the Cornified Envelope,” Br. J. Dermatol. 152:146-9 (2005) and Guttman-Yassky et al., “Broad Defects in Epidermal Cornification in Atopic Dermatitis Identified Through Genomic Analysis,” J. Allergy Clin. Immunol. 124:1235-44 e58 (2009), which are hereby incorporated by reference in their entirety. Of note, FLG was down-regulated in AD vs. NA (Z ratio: −5.29) however, the P value of 0.01 did not reach the significant threshold. Using the expression profiling array data from NA controls it was verified that human keratinocytes express claudins-1, -4, -8, -12 and -14 (Brandner, J. M., “Tight Junctions and Tight Junction Proteins in Mammalian Epidermis,” Eur. J. Pharm. Biopharm. 72:289-94 (2009); Brandner et al., “Organization and Formation of the Tight Junction System in Human Epidermis and Cultured Keratinocytes,” Eur. J. Cell Biol. 81:253-63 (2002); and Watson et al., “Altered Claudin Expression is a Feature of Chronic Plaque Psoriasis,” J. Pathol. 212:450-8 (2007), which are hereby incorporated by reference in their entirety) and demonstrated for the first time that they also express claudins-15 and -23 (Illumina detection values ≧0.99). Interestingly, of the intracellular junction genes, claudin-1 met the criteria for significance, demonstrating reduced expression in AD subjects with a Z ratio of −2.4 and P=0.0013 compared to NA (FIG. 2A—highlights in Gap Junctions and Tight Junctions rows). No difference was observed in claudin-1 expression in psoriasis as compared to NA control subjects (P=0.98) (FIG. 2A—highlights in Gap Junctions and Tight Junctions rows). Reduced expression of claudin-23 (Z ratio: −3.2; P=0.0062) in AD epithelium was also noted compared to NA, but the difference was only marginally significant (FIG. 2A—highlights in Gap Junctions and Tight Junctions rows) (see also FIGS. 4A and 4B). Furthermore, AD subjects displayed enhanced expression of the gap junction proteins connexin-26 (GJB2, Z ratio: +5.2; P=0.025) and connexin-62 (GJA10, Z ratio: +1.6; P=0.001) (FIG. 2A-highlights in Gap Junctions and Tight Junctions rows). AD subjects did not show decreased expression of proteins relevant for adherens junctions or desmosomes. Importantly, the expression of a number of differentiation genes were either not affected or increased in AD compared to controls (FIG. 2A-highlights in Differentiation Markers row) indicating that the observed changes in TJ genes did not simply reflect dedifferentiation of the epidermis. The reduced expression of claudin-1 was confirmed in blister roofs obtained from newly recruited AD and NA subjects (n=5 per group) by qPCR (AD: 38.4±7.1 RVU vs. NA: 69.7±9.5; P=0.03) (FIG. 4A). The reduced expression of claudin-23 (AD: 0.23±0.04 RVU vs. NA: 0.81±0.12; P=0.001) and the enhanced expression of connexin-26 (GBJ2, AD: 18.87±6.7 vs. NA: 3.73±0.26; P=0.03) was also confirmed in AD vs. NA controls by qPCR (FIG. 4B).

In order to investigate claudin-1 expression in intact skin, skin biopsies from nonlesional AD skin was compared to NA using both immunohistochemistry (FIGS. 5A, 5B) and immunofluorescent/confocal microscopy (FIGS. 5C, 5D). Claudin-1 immunoreactivity was detectable in brightfield images in all suprabasal layers, while occludin and ZO-1 were detected only in the upper granulosum layer where TJ form. This pattern as has been noted by other investigators. Schluter et al., “The Different Structures Containing Tight Junction Proteins in Epidermal and Other Stratified Epithelial Cells, Including Squamous Cell Metaplasia,” Eur. J. Cell. Biol. 86(11-12):645-55 (2007) and Langbein et al., “Tight Junctions and Compositionally Related Junctional Structures in Mammalian Stratified Epithelia and Cell Cultures Derived Therefrom,” Eur. J. Cell Biol. 81:419-35 (2002), which are hereby incorporated by reference in their entirety. Importantly, the expression of claudin-1 was markedly reduced in nonlesional skin from subjects with AD compared to NA controls (FIGS. 5A-5D). Semiquantitative claudin-1 scoring confirmed the markedly reduced staining (>50%) in the epidermis of AD (1.3±0.3) compared to NA subjects (2.9±0.1; P<0.0004) and the highly significant P value demonstrates the remarkable uniformity of staining intensity within each group (FIG. 5E). Immunofluorescent staining more clearly demonstrated claudin-1 immunoreactivity on the cell membranes. The signal intensity was again significantly less in AD samples (FIGS. 5C, 5D). These findings provide the first indication that the skin barrier defect in AD subjects may reside below the level of the stratum corneum (“SC”), at the level of TJs.

### Example 2

### AD Epidermis Shows Defective Bioelectric Properties

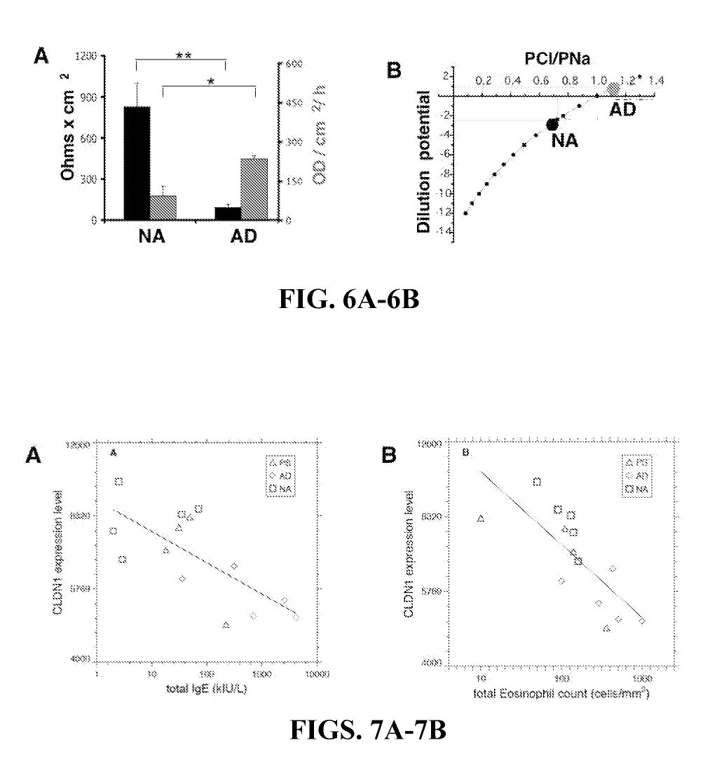

The functional impairment of TJ barrier in AD epidermis was next investigated by measuring bioelectric characteristics in Ussing chambers. This approach has been used in human and mouse models to evaluate the TJ bioelectric property of mucosal epithelia. Madara et al., “Regulation of the Movement of Solutes Across Tight Junctions,” Annu. Rev. Physiol. 60:143-59 (1998) and Schmitz et al., “Altered Tight Junction Structure Contributes to the Impaired Epithelial Barrier Function in Ulcerative Colitis,” Gastroenterology 116:301-9 (1999), which are hereby incorporated by reference in their entirety. The technique is based on the principle that an intact semi-permeable membrane will maintain the electrochemical potential gradient generated artificially by bathing each side of the epidermal sheets with solutions of different ionic strength. Ussing and Zerahn, “Active Transport of Sodium as the Source of Electric Current in the Short-Circuited Isolated Frog Skin,” Acta Physiol. Scand. 23:110-27 (1951) and Van de Kerkhof et al., “In Vitro Methods to Study Intestinal Drug Metabolism,” Curr. Drug Metab. 8:658-75 (2007), which are hereby incorporated by reference in their entirety. A leaky membrane will allow easy diffusion across the membrane and thus loss of electrochemical potential. Thus, the higher the permeability across a membrane the lower the potential gradient. Schmitz et al., “Altered Tight Junction Structure Contributes to the Impaired Epithelial Barrier Function in Ulcerative Colitis,” Gastroenterology 116:301-9 (1999) and Wang et al., “Human Zonulin, a Potential Modulator of Intestinal Tight Junctions,” J. Cell Sci. 113 Pt 24:4435-40 (2000), which are hereby incorporated by reference in their entirety. TEERs were stable and typically quite high—indicating intact paracellular barrier function of the electrically active epithelium. Using this approach, AD epidermis showed a dramatically lower resistance (92±22.0 Ohms×cm²; n=3) as compared to NA subjects (827±173.3 Ohms×cm²; P=0.01; n=4) (FIG. 6A). The lower resistance observed in the Ussing chambers was consistent with the increased permeability of FITC-conjugated albumin in AD (445±24.25 O.D./cm²/h; n=3) as compared to NA samples (175±68.37 O.D./cm²/h; P=0.02; n=4) (FIG. 6A).

To better investigate the paracellular permeability properties, the relative permeability of Cl⁻ and Na⁺ (P_{Cl}/P_{Na}) was measured. Vidyasagar and Ramakrishna, “Effects of Butyrate on Active Sodium and Chloride Transport in Rat and Rabbit Distal Colon,” J. Physiol. 539:163-73 (2002), which is hereby incorporated by reference in its entirety. Because AD subjects have lower BPs than age and sex matched controls (Uehara M, Sugiura H, Tanaka K. Rarity of hypertension in adult patients with atopic dermatitis. Br J Dermatol. 146(4):631-5 (2002), which is hereby incorporated by reference in its entirety), it is conceivable that this TJ bioelectric defect could explain the lack of selectivity for Na⁺ versus Cl⁻ ions. If P_{Cl}/P_{Na} is equal to one then there is no selectivity and the membrane is freely permeable. Dilution potential studies on epidermal sheets showed the membrane selectivity is preserved in skin from NA subjects, with Na⁺ ions relatively more permeable than Cl⁻ (0.77±0.03 fold P_{Cl}/P_{Na}). However, in AD subjects' samples the selectivity was completely lost (1.1±0.02 fold P_{Cl}/P_{Na}; P=0.001; n=3/group) as both ions were equally permeable (FIG. 6B).

### Example 3

### Claudin-1 Expression is Inversely Correlated with Th2 Biomarkers

To address whether expression levels of epidermal claudin-1 might modulate adaptive immune responses, the relationship between claudin-1 mRNA expression in the epidermal samples from AD, PS, and NA controls and biomarkers of T helper (“Th”) 2 polarity, namely serum total IgE and peripheral blood eosinophilia (FIGS. 7A, 7B), was examined Claudin-1 levels were inversely correlated with both total IgE (r=−0.718; P=0.0038) and Eos (r=−0.761; P=0.0016) suggesting that reductions in this key TJ barrier protein may affect the character of the immune response to environmental allergens or vice versa.

### Example 4

### Claudin-1 Localizes to TJ Only in Differentiated Human Keratinocytes

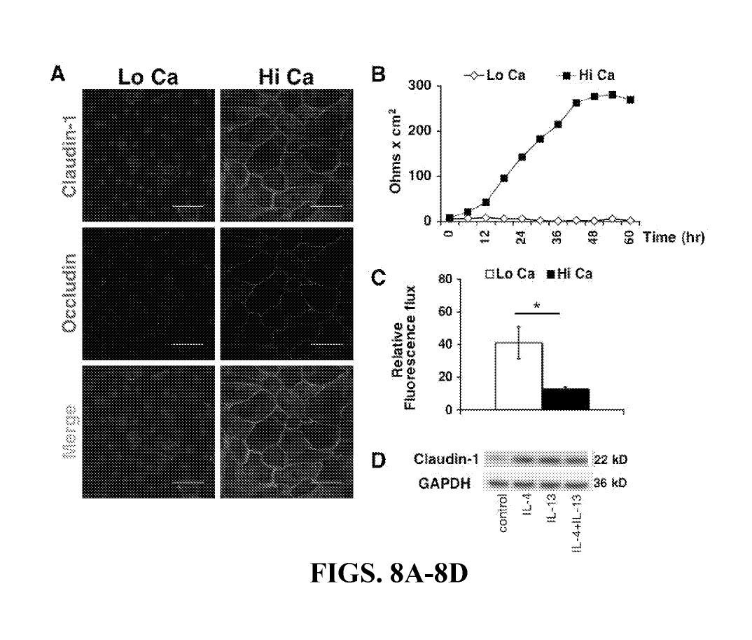

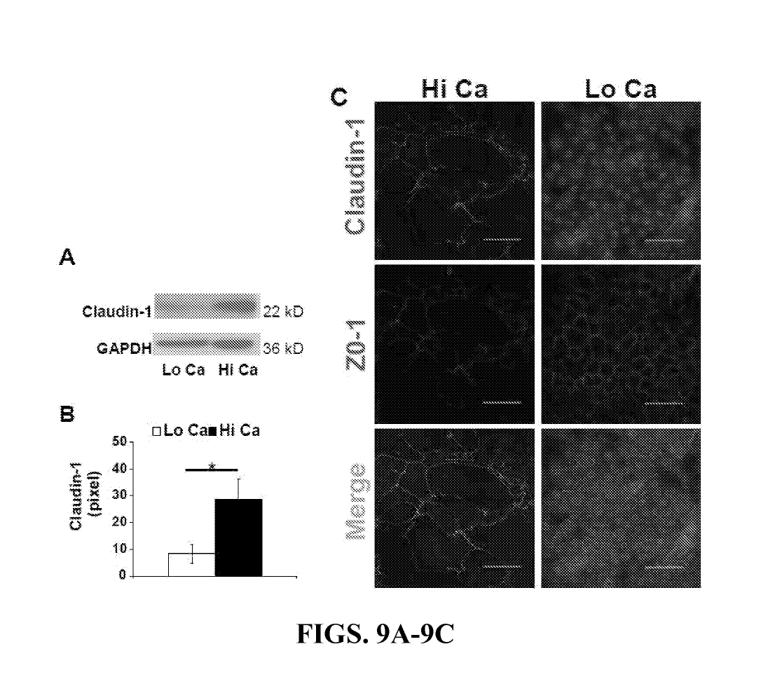

Claudin-1 protein is expressed in primary human keratinocytes (“PHK”) in vitro mainly when PHK were differentiated in high (1.9 mM; Hi Ca) versus low (0.3 mM; Lo Ca) Ca⁺² containing media for 24 h after confluency (FIGS. 9A-B) Claudin-1 colocalizes with other TJ proteins at the cell membrane only after Ca⁺²-induced differentiation (FIGS. 8A-D; 9A-C). Confocal microscopy demonstrated that claudin-1 colocalizes with occludin and ZO-1 at the areas of cell-cell contacts only in PHK grown in the media with high concentration of Ca⁺² (Hi Ca; 1.9 mM) for at least 24 h after confluency. Interestingly, in undifferentiated PHK grown in 0.3 mM Ca⁺² (Lo Ca) containing media, claudin-1 immunoreactivity was faint and largely nuclear or perinuclear, while occludin was undetectable (FIG. 8A) and ZO-1 localized on the membrane with a discontinuous pattern (FIG. 9C).

Next, the functional relevance of these Ca⁺²-induced changes in TJ protein expression and localization was investigated. Measurements of trans-epithelial electric resistance (“TEER”) and molecular marker fluxes are routinely used to evaluate TJ integrity. Utech et al., “Tight Junctions and Cell-Cell Interactions,” Methods Mol. Biol. 341:185-95 (2006), which is hereby incorporated by reference in its entirety. As noted above, TEER reflects paracellular permeability of small ions, whereas fluxes indicate TJ permeability to larger molecules.

It was observed that during Ca⁺²-induced PHK differentiation a dramatic (−300 fold) increase in TEER which peaked between 30 to 60 h after exposure to high extracellular Ca⁺² (FIG. 8B), and confirmed previously published studies. Furuse et al., “Claudin-Based Tight Junctions Are Crucial for the Mammalian Epidermal Barrier: A Lesson From Claudin-1-Deficient Mice,” J. Cell Biol. 156:1099-111 (2002); Yamamoto et al., “Effect of RNA Interference of Tight Junction-Related Molecules on Intercellular Barrier Function in Cultured Human Keratinocytes,” Arch. Dermatol. Res. 300:517-24 (2008); Yamamoto et al., “Relationship Between Expression of Tight Junction-Related Molecules and Perturbed Epidermal Barrier Function in UVB-Irradiated Hairless Mice,” Arch. Dermatol. Res. 300:61-8 (2008); Tsukita et al., “Tight Junction-Based Epithelial Microenvironment and Cell Proliferation,” Oncogene 27:6930-8 (2008); Lopardo et al., “Claudin-1 is a p63 Target Gene With a Crucial Role in Epithelial Development,” PLoS One 3:e2715 (2008); and Kurasawa et al., “Regulation of Tight Junction Permeability by Sodium Caprate in Human Keratinocytes and Reconstructed Epidermis,” Biochem. Biophys. Res. Commun. 381:171-5 (2009), which are hereby incorporated by reference in their entirety. Additionally, it was shown that sodium fluorescein flux was markedly reduced in differentiated PHK (3.1±0.6 fold; P=0.046) (FIG. 8C). Importantly, the development of a paracellular barrier in differentiated PHK monolayers coincided with the appearance of claudin-1 at the areas of cell-cell contacts (FIG. 8A), and therefore indicates that these events may be causally connected.

### Example 5

### Th2 Cytokines Enhance Claudin-1 Expression and TJ barrier Function

Previous studies have shown that claudin-1 expression in noncutaneous epithelial cells can be modulated by cytokines and growth factors. Kinugasa et al., “Claudins Regulate the Intestinal Barrier in Response to Immune Mediators,” Gastroenterology 118:1001-11 (2000); Tedelind et al., “Interferon-Gamma Down-Regulates Claudin-1 and Impairs the Epithelial Barrier Function in Primary Cultured Human Thyrocytes,” Eur. J. Endocrinol. 149:215-21 (2003); and Singh and Harris, “Epidermal Growth Factor Receptor Activation Differentially Regulates Claudin Expression and Enhances Transepithelial Resistance in Madin-Darby Canine Kidney Cells,” J. Biol. Chem. 279:3543-52 (2004), which are hereby incorporated by reference in their entirety. Recent findings from mouse models support the hypothesis that IL-4 could delay skin barrier recovery. Kurahashi et al., “IL-4 Suppresses the Recovery of Cutaneous Permeability Barrier Functions In Vivo,” J. Invest. Dermatol. 128:1329-31 (2008) and Sehra, et al., “IL-4 Regulates Skin Homeostasis and the Predisposition Toward Allergic Skin Inflammation,” J. Immunol. 184:3186-90 (2010), which are hereby incorporated by reference in their entirety. It was therefore considered if claudin-1 down-regulation observed in AD nonlesional skin biopsies could be secondary to Th2 cytokines. A corollary to this hypothesis is that even nonlesional skin has been shown to express Th2 cytokines. Hamid et al., “Differential In Situ Cytokine Gene Expression in Acute Versus Chronic Atopic Dermatitis,” J. Clin. Invest. 94:870-6 (1994), which is hereby incorporated by reference in its entirety. To test this possibility, PHK were differentiated in the presence or absence of Th2 cytokines, IL-4 and IL-13, and the expression of claudin-1, occludin and ZO-1 was evaluated. It was observed that a significant enhancement of claudin-1 expression after IL-4 (48 hours, 50 ng/ml; 2.1±0.4 fold over control; P=0.05; n=3) and IL-13 stimulation (48 hours, 50 ng/ml; 1.6±0.3 fold over control; P=0.1; n=3; FIG. 8D). No synergism was noted with both cytokines and interestingly no effect was observed on the expression of occludin and ZO-1. The effect of IL-4 and IL-13 on PHK barrier function was then investigated. A significant increase in TEER peak was observed in IL-4 (50 ng/ml) treated PHK (166±24 Ohms×cm² vs. media alone 109±11 Ohms×cm²; P=0.03; n=4). Dose response experiments confirmed IL-4's effect on TEER (dose range 0.5-100 ng/ml), while IL-13 (0.5-100 ng/ml) induced only a slight increase in TEER. The mechanisms by which Th2 cytokines paradoxically enhance barrier function in the primary human keratinocytes is currently unknown, but these data indicate that the reduced claudin-1 expression and TJ dysfunction observed in AD subjects are not due to the actions of Th2 cytokines. However, at this point the possibility that the impaired barrier observed in AD epidermis is a consequence of the Th2 cytokines present in the underlying dermis cannot be excluded.

### Example 6

### Claudin-1 Knockdown Disrupts TJ Function

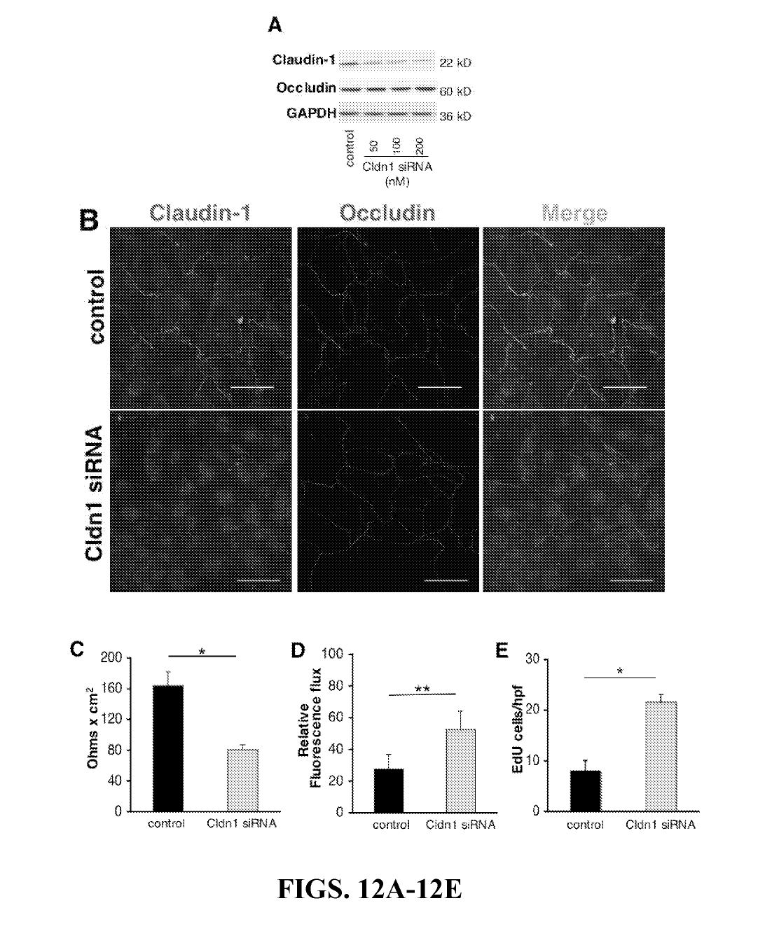

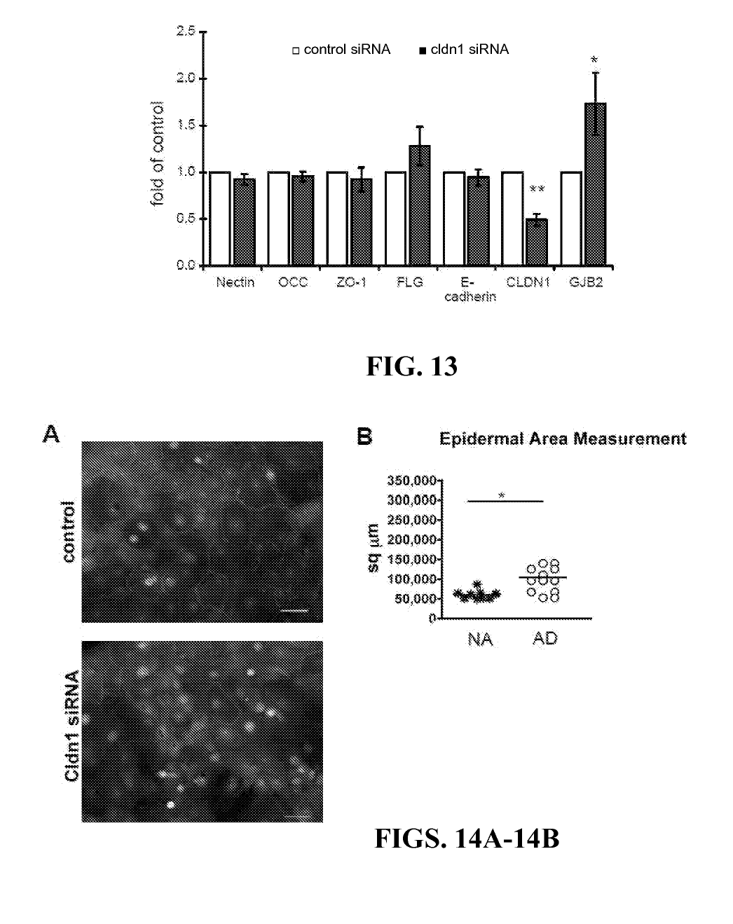

An RNA interference approach was utilized to evaluate the effect that claudin-1 reduction has on measures of TJ function (TEER and permeability) and proliferation. FIG. 12A shows that claudin-1-specific siRNA induced a dose-dependent and significant (≦60%) decrease in protein expression compared to scrambled siRNA-transfected cells. Since the claudin-1 KO mouse dies shortly after birth and the tissue staining demonstrated ˜50% reduction in claudin-1 immunoreactivity in AD compared to NA epithelium (FIG. 5), functional assessments were performed with a dose of siRNA (100 nM) that reduced claudin-1 transcripts by a similar amount (P=0.01). With only a 50% reduction in claudin-1, TEER dropped by ≧50% (FIG. 12C) (control: 164±18.2 and CLDN1 siRNA: 80.6±6.4 ohms×cm²; P=0.007; n=4) and permeability to sodium fluorescein increased by a similar extent (FIG. 12D) (control: 27.6±11.7 and CLDN1 siRNA: 52.5±9.2; P=0.026; n=4) when compared to the scrambled siRNA-transfected PHK. Yamamoto et al. used a similar knockdown strategy, but observed a more modest effect (14% reduction) on TEER and did not assess TJ function using a permeability assay. Yamamoto et al., “Effect of RNA Interference of Tight Junction-Related Molecules on Intercellular Barrier Function in Cultured Human Keratinocytes,” Arch. Dermatol. Res. 300:517-24 (2008), which is hereby incorporated by reference in its entirety. The effect of claudin-1 knockdown on connexin-26 (GJB2) was also investigated, since this was upregulated in AD array samples. Enhanced expression of connexin-26 was observed in the knockdowns (1.7±0.8 fold over control; P=0.05; n=6; FIG. 13).

Using a proliferation assay based on EdU incorporation, it was noted that claudin-1 depleted cells have more proliferating cells (21.6±1.4 EdU positive cells/hpf) compared to control (8.1±2 EdU positive cells/hpf; P=0.002; n=3) (FIG. 12E).

### Example 7

### CLDN1 Variants are Associated with Risk of AD

Because these findings strongly implicated claudin-1 as a critical element in risk of AD, the ongoing NIAID-funded Atopic Dermatitis and Vaccinia Network (“ADVN”) was used, which is currently enrolling subjects with AD and NA (reviewed in Beck et al., “Phenotype of Atopic Dermatitis Subjects With a History of Eczema Herpeticum,” J. Allergy Clin. Immunol. 124:260-9, 9 e1-7 (2009), which is hereby incorporated by reference in its entirety) to examine whether common variants in the human CLDN1 gene might be associated with susceptibility to AD and disease severity. Two independent groups (European American, African American) of AD patients and healthy controls were enrolled (Table 3).

TABLE 3
ADVN Genetics Study Demographics

| | European American | | African American | |
|---|---|---|---|---|
| Characteristic | AD | NA | AD | NA |
| Sample size | 258 | 156 | 176 | 152 |
| Males; | 96 | 63 | 43 | 77 |
| N (%) | (37.2%) | (40.4%) | (24.4%) | (50.7%) |
| Age; | 33.1 | 36.6 | 35.3 | 41.1 |
| mean (SD) | (18.5) | (13.2) | (12.5) | (10.3) |
| AD onset < 5yrs; | 178 | NA | 91 | NA |
| N (%) | (68.9%) | | (51.7%) | |
| Geometric mean IgE levels; | 670.0 | 59.1 | 556.8 | 141.2 |
| (95% CI) | (502-895) | (48-111) | (425-729) | (113-299) |
| Geometric mean EASI†; | 4.6 | NA | 4.0 | NA |
| (95% CI) | (3.9-5.4) | | (3.3-4.9) | |
| FLG Null Allele Carrier; | 65 | 9 | 11 | 2 |
| N (%) | (25.2%) | (5.8%) | (6.2%) | (1.3%) |

The following abbreviations are used: AD, atopic dermatitis; EASI, Eczema area and severity index; and NA, not applicable.
†EASI determined by the percentage of eczema area on a 7-point ordinal scale: 0 = <10%; 1 = 10%-29%; 3 = 30%-49%; 4 = 50%-69%; 5 = 70%-89%; and 6 = 90%-100

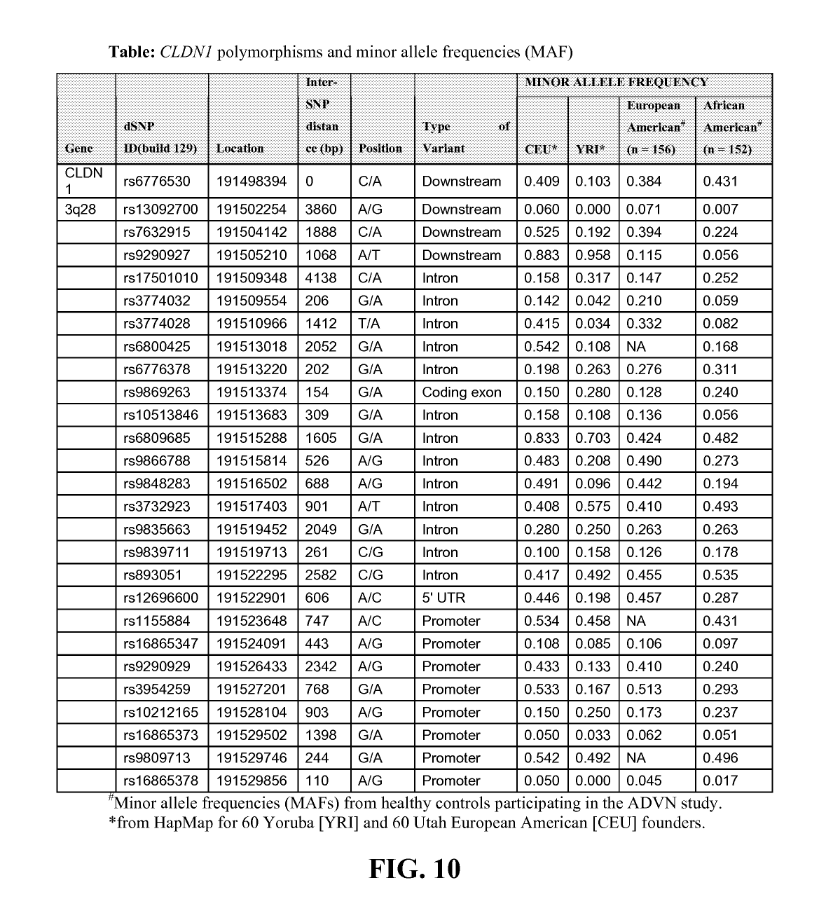

In brief, AD was diagnosed using the US consensus conference criteria36, and AD severity was defined according to the ‘eczema area and severity index’ (EAST), a standardized grading system46. Twenty-seven CLDN1 SNPs spanning a 31.5 kb region on chromosome 3q28-q29 were selected using a haplotype-tagging approach, of which 24 were common to both ethnic groups (see FIG. 10).

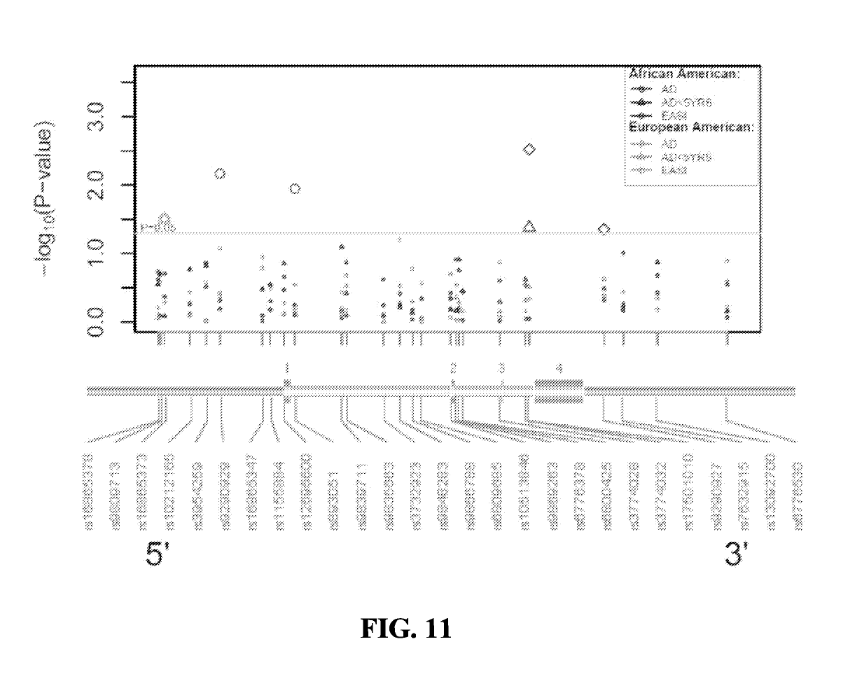

Adjusting for 10,000 permutations, the most significant associations for a lower risk of AD were observed in the African American group for an intronic SNP (rs17501010) between the 3^{rd} and 4^{th} exons of CLDN1 ([OR], 0.5, 95% CI=0.3-0.8; P=0.003) and an adjacent SNP (rs9290927) downstream of rs17501010 that was associated with a higher risk of AD ([OR], 1.8, 95% CI=1.0-3.3; P=0.004) (FIG. 11 and Table 4).

TABLE 4
CLDN1 SNP Association Test Results

| dbSNP ID | European American | | | African American | | |
|---|---|---|---|---|---|---|
| (build 129) | AD | AD <5 years | EASI | AD | AD <5 years | EASI |
| rs6776530 | 0.25 | 0.13 | 0.61 | 0.86 | 0.7 | 0.28 |
| rs13092700 | 0.66 | 0.45 | 0.42 | 0.21 | 0.13 | 0.37 |
| rs7632915 | 0.53 | 0.36 | 0.54 | 0.67 | 0.58 | 0.1 |
| rs9290927 | 0.41 | 0.24 | 0.44 | 0.04 | 0.33 | 0.49 |
| | | | | (0.039*) | | |
| rs17501010 | 0.46 | 0.31 | 0.92 | 0.003 | 0.04 | 0.91 |
| | | | | (0.005*) | (0.065*) | |
| rs3774032 | 0.48 | 0.48 | 0.7 | 0.29 | 0.24 | 0.9 |
| rs3774028 | 0.84 | 0.7 | 0.13 | 0.96 | 0.51 | 0.25 |
| rs6800425 | NA | NA | 0.69 | 0.36 | 0.36 | 0.94 |
| rs6776378 | 0.7 | 0.52 | 0.94 | 0.18 | 0.12 | 0.62 |
| rs9869263 | 0.29 | 0.44 | 0.94 | 0.68 | 0.69 | 0.12 |
| rs10513846 | 0.46 | 0.2 | 0.86 | 0.46 | 0.65 | 0.38 |
| rs6809685 | 0.99 | 0.93 | 0.27 | 0.89 | 0.46 | NA |
| rs9866788 | 0.5 | 0.74 | 0.17 | 0.67 | 0.75 | 0.93 |
| rs9848283 | 0.52 | 0.29 | 0.06 | 0.38 | 0.61 | 0.57 |
| rs3732923 | 0.99 | 0.99 | 0.77 | 0.58 | 0.82 | 0.24 |
| rs9835663 | 0.21 | 0.29 | 0.68 | 0.81 | 0.38 | 0.13 |
| rs9839711 | 0.64 | 0.75 | 0.37 | 0.7 | 0.08 | 0.89 |
| rs893051 | 0.73 | 0.64 | 0.29 | 0.57 | 0.79 | 0.01 |
| | | | | | | (0.010*) |
| rs12696600 | 0.78 | 0.75 | 0.53 | 0.34 | 0.14 | 0.22 |
| rs1155884 | NA | NA | 0.32 | 0.66 | 0.29 | 0.49 |
| rs16865347 | 0.11 | 0.16 | 0.34 | 0.81 | 0.33 | 0.96 |
| rs9290929 | 0.38 | 0.42 | 0.08 | 0.49 | 0.64 | 0.007 |
| | | | | | | (0.006*) |
| rs3954259 | 0.94 | 0.97 | 0.27 | 0.31 | 0.15 | 0.14 |
| rs10212165 | 0.52 | 0.81 | 0.38 | 0.76 | 0.17 | 0.55 |
| rs16865373 | 0.03 | 0.03 | 0.52 | 0.43 | 0.82 | 0.2 |
| | (0.034*) | (0.034*) | | | | |
| rs9809713 | NA | NA | 0.26 | 0.82 | 0.2 | 0.2 |
| rs16865378 | 0.77 | 0.89 | 0.62 | 0.19 | 0.28 | 0.24 |

*Empiric p-value based on 10,000 permutations

SNP-rs17501010 also showed a modest association with early onset AD (<5 yrs of age—P=0.04). In addition, two SNPs (rs893051 in intron 1 and rs9290929 in the promoter region) were associated with greater disease severity ([OR], 1.5, 95% CI=1.1-2.1 P=0.010; 1.6, 95% CI=1.1-2.3 P=0.007, respectively; FIG. 11 and Table 4). Modest associations were observed in the European American group, including a promoter SNP (rs16865373) and lower risk of AD ([OR], 0.5, 95% CI=0.2-0.9; P=0.034) and lower risk of early onset AD (<5 yrs of age -[OR], 0.4, 95% CI=0.2-1.0; P=0.034; FIG. 11 and Table 4). Haplotype analyses were performed, but did not alter the evidence for association for any of the outcomes, including FLG null mutation (e.g., R501X and 2282de14) phenotype. Considering the importance of FLG mutations in AD and its subphenotypes, it was also evaluated whether there was an interaction between haplotype-tagging SNPs in FLG and CLDN1. No significant interaction effect was found. Despite the limitations of relatively small sample sizes, these findings suggest that CLDN1 genetic variations may determine risk for and severity of AD in ethnically diverse populations and that this appears to be independent of FLG. This is the first study to implicate a TJ protein and CLDN1 in particular, as a strong candidate gene for AD.

### Discussion of Examples 1-7

The preceding examples for the first time implicate a TJ defect in AD, a human skin disease that affects up to 15 million Americans. Reduced expression of epidermal claudin-1 in AD nonlesional epidermis is demonstrated (FIGS. 5A-E). This was specific for AD and not observed in psoriasis, a Th17-driven inflammatory skin disorder (FIGS. 2A-D). Although previous psoriasis publications have suggested that TJ may be altered in lesional epidermis this has not been consistently observed by other groups. Watson et al., “Altered Claudin Expression is a Feature of Chronic Plaque Psoriasis,” J. Pathol. 212:450-8 (2007); Peltonen et al., “Tight Junction Components Occludin, ZO-1, and Claudin-1, -4 and -5 in Active and Healing Psoriasis,” Br. J. Dermatol. 156:466-72 (2007); Kirschner et al., “Alteration of Tight Junction Proteins is an Early Event in Psoriasis: Putative Involvement of Proinflammatory Cytokines,” Am. J. Pathol. 175:1095-106 (2009); and Itoh et al., “Identification of Differentially Expressed Genes in Psoriasis Using Expression Profiling Approaches,” Exp. Dermatol. 14:667-74 (2005), which are hereby incorporated by reference in their entirety.

Adapting the Ussing chamber to measure bioelectric properties of stratified squamous epithelium enabled characterization of skin barrier properties from human subjects. Using this approach a remarkable alteration in the bioelectric characteristics of AD epidermis with markedly lower electrical resistance and higher albumin permeability that was associated with the loss of ion selectivity permeability was observed (FIGS. 6A, B). These defects are the signature of a TJ defect.

These findings are highly consistent with the observations made from genetically altered mice. For example, the claudin-1 knockout mouse dies within 24 hr of birth with wrinkled skin, severe dehydration and increased epidermal permeability as measured by dye studies and TEWL. Furuse et al., “Claudin-Based Tight Junctions Are Crucial for the Mammalian Epidermal Barrier: A Lesson From Claudin-1-Deficient Mice,” J. Cell Biol. 156:1099-111 (2002), which is hereby incorporated by reference in its entirety. Importantly, these mice have no abnormalities in the expression of stratum corneum proteins (e.g., loricrin, involucrin, transglutaminase-1, or Klf4) or lipids that might explain their severe skin phenotype. Another recent study reported disruption of epidermal barrier and severe dermatitis in transgenic mice overexpressing an adhesion-deficient mutant of claudin-6 in the suprabasal compartment of the skin. Troy et al., “Dermatitis and Aging-Related Barrier Dysfunction in Transgenic Mice Overexpressing an Epidermal-Targeted Claudin 6 Tail Deletion Mutant,” PLoS One 4:e7814 (2009), which is hereby incorporated by reference in its entirety. Interestingly, a marked downregulation of claudin-1 expression was noted in these transgenic mice. Lopardo et al. also observed reduced claudin-1 expression in the epidermis of p63 mutant mice. Lopardo et al., “Claudin-1 is a p63 Target Gene With a Crucial Role in Epithelial Development,” PLoS One 3:e2715 (2008), which is hereby incorporated by reference in its entirety. These mice have a severe skin phenotype and die of dehydration within one day of birth similar to the claudin-1 KO mice. In summary, these mouse models have highlighted the critical importance of claudin-1 for a functional epidermal TJ. Recently, a human syndrome caused by an exonic mutation in the CLDN1 has been described. Hadj-Rabia et al., “Claudin-1 Gene Mutations in Neonatal Sclerosing Cholangitis Associated with Ichthyosis: A Tight Junction Disease,” Gastroenterology 127:1386-90 (2004); Feldmeyer et al., “Confirmation of the Origin of NISCH Syndrome,” Hum. Mutat. 27:408-10 (2006); and Zimmerli et al., “Human Epidermal Langerhans Cells Express the Tight Junction Protein Claudin-1 and Are Present in Human Genetic Claudin-1 Deficiency (NISCH Syndrome),” Exp. Dermatol. 17:20-3 (2008), which are hereby incorporated by reference in their entirety. Patients with this syndrome called Neonatal Ichthyosis-Sclerosing Cholangitis (NISCH) have features in common with AD, namely erythema, dry flaky skin and patchy alopecia in addition to unique features such as severe liver and gallbladder abnormalities that likely arise because of the importance of claudin-1 in the barrier integrity of bile canaliculi. Hadj-Rabia et al., “Claudin-1 Gene Mutations in Neonatal Sclerosing Cholangitis Associated with Ichthyosis: A Tight Junction Disease,” Gastroenterology 127:1386-90 (2004); Feldmeyer et al., “Confirmation of the Origin of NISCH Syndrome,” Hum. Mutat. 27:408-10 (2006), which are hereby incorporated by reference in their entirety.

The in vitro studies with PHK monolayers demonstrate a clear association between claudin-1 levels and TEER. These findings were extended by demonstrating that claudin-1 knockdown also enhanced TJ permeability and proliferation (FIGS. 12A-E). In this study, the claudin-1 siRNA was target-specific, with no changes observed in the expression and/or localization of other critical TJ proteins (occludin and ZO-1), adherens junction components (E-cadherin and nectin-1) or stratum corneum proteins (filaggrin) (FIG. 13). Additionally, claudin-1 expression was reduced by ˜50%—similar to the reductions observed in claudin-1 immunoreactivity of AD epidermis and this resulted in a remarkable reduction (−50%) in TJ function (based on both electrical resistance and permeability assays) (FIGS. 12C-D). While immunolabeling data allow only a correlative link between claudin-1 expression and the development of epidermal barrier, the RNA interference results demonstrate a causal connection between these two events (FIGS. 12A-E).

Recent studies with different types of mammalian epithelia indicated that the epithelial barrier is determined by two different components, charge-selective small pores that are permeable to molecules with up to 4μ radius, and large, poorly-defined breaks of the barrier without size- or charge-selectivity (reviewed in Anderson and Van Itallie, “Physiology and Function of the Tight Junction,” Cold Spring Harbor Perspect. Biol. 1:a002584 (2009), which is hereby incorporated by reference in its entirety). TEER reflects small-pore permeability, whereas flux of FITC with a reported Stokes' radius of ˜5.5 Å measures permeability of large barrier breaks. Knockdown of claudin-1 in PHK monolayers decreased TEER and increased FITC flux, which implicates claudin-1 in regulation of both paracellular pores and breaks in the epidermal barrier. These findings strongly indicate that the skin of AD subjects would also be more permissive to a number of relevant environmental allergens. Purified allergens from the house dust mite, Dermatophagoides pteronyssinus have a diameter of ˜1.6 Å by X-ray crystallography and therefore could penetrate even small paracellular pores formed by epithelial TJs. Meno et al., “The Crystal Structure of Recombinant proper p 1, A Major House Dust Mite Proteolytic Allergen,” J. Immunol. 175:3835-45 (2005), which is hereby incorporated by reference in its entirety. Thus, defective expression and function of claudin-1 in AD provides a plausible molecular mechanism for increased sensitization to environmental antigens, allergens, irritants, or pollutants. This is in keeping with the distinction that AD is the allergic disorder with the greatest and most diverse allergen reactivity, reflected in high serum total IgE values. AD is also recognized for a reduced cutaneous irritancy threshold that could simply reflect greater epidermal penetration of irritants. Importantly, TJ disruption is a leading hypothesis to explain allergen reactivity in the airways, which manifests as asthma and allergic rhinitis or in the intestinal tract as food allergy. Schulzke et al., “Epithelial Tight Junctions in Intestinal Inflammation,” Ann. N.Y. Acad. Sci. 1165:294-300 (2009); Runswick et al., “Pollen Proteolytic Enzymes Degrade Tight Junctions,” Respirology 12:834-42 (2007); Groschwitz and Hogan, “Intestinal Barrier Function: Molecular Regulation and Disease Pathogenesis,” J. Allergy Clin. Immunol. 124:3-20; quiz 1-2 (2009), which are hereby incorporated by reference in their entirety.

Although it was observed that an inverse correlation between claudin-1 expression and markers of Th2 polarity, it was not found that Th2 cytokines (IL-4 and IL-13 alone or together) reduced claudin-1 expression. Rather, the opposite was observed. This induction of claudin-1 was observed in conjunction with enhanced TJ function (e.g. TEER) and indicates that Th2 cytokines have a reparative effect on TJ in normal keratinocytes. Interestingly, Th2 cytokines have been shown to reduce the expression of several SC components important for skin barrier function. Sehra, et al., “IL-4 Regulates Skin Homeostasis and the Predisposition Toward Allergic Skin Inflammation,” J. Immunol. 184:3186-90 (2010), which is hereby incorporated by reference in its entirety. At this point it is reasonable to conclude that TJ dysfunction observed in AD epidermis is not likely caused by Th2 milieu, which is present even at nonlesional sites. Instead the connection observed herein between TJ function and biomarkers of Th2 polarity indicates that AD TJ defects enable or promote Th2 responses, possibly by enhancing the trafficking of non-self antigens that are responsible for triggering the Th2 response in genetically predisposed individuals. Alternatively, the upregulation of claudin-1 in response to IL-4 and IL-13 may represent a compensatory immune response to “protect” against further antigen uptake through the skin.

In preliminary studies, genetic associations between CLDN1 polymorphisms and AD were evaluated. A haplotype-tagging SNP approach was undertaken using genetic markers available in the public arena (n=132 in dbSNP) in 414 European Americans and 328 African Americans. Interestingly, CLDN1 is localized on chromosome 3q28-q29, very close to the ATOD1 locus for AD. Morar et al., “The Genetics of Atopic Dermatitis,” J. Allergy Clin. Immunol. 118:24-34; quiz 5-6 (2006), which is hereby incorporated by reference in its entirety. Adjusting for 10,000 permutations to reduce a Type I error due to multiple comparisons, several modest associations (P=0.003-0.05) were observed between variants throughout the CLDN1 gene and the outcomes associated with AD, especially among African American patients. In separate studies association between several of the same CLDN1 SNPs and risk of disease among a more robustly powered German dataset of AD patients and controls was tested; however, the full set of SNPs genotyped in the ADVN study were not available for direct comparisons, and phenotyping approaches differed. Further studies are underway for comprehensive joint analyses between ADVN and the German study. Interestingly, CLDN1 variants were also associated with asthma and its related trait, total serum IgE and FEVₛ, in two independent populations of African descent. In addition, the rs893051 SNP associated with AD severity in the AA population, was also associated with asthma and disease severity in a population of African descent. As part of the ADVN study, the instant population was also screened for the two most frequent FLG mutations (R501X and 2282de14; Table 3) and 9 haplotype-tagging SNPs throughout the FLG gene. Gao et al., “Filaggrin Mutations That Confer Risk of Atopic Dermatitis Confer Greater Risk for Eczema Herpeticum,” J. Allergy Clin. Immunol. 124:507-13, 13 e1-7 (2009), which is hereby incorporated by reference in its entirety. There were no significant interaction effects between haplotype-tagging SNPs in FLG and CLDN1 SNPs. Moreover, in the AA population that had the strongest association with CLDN1 SNPs, the 2 FLG mutations were considerable less common than in the EA population. Gao et al., “Filaggrin Mutations That Confer Risk of Atopic Dermatitis Confer Greater Risk for Eczema Herpeticum,” J. Allergy Clin. Immunol. 124:507-13, 13 e1-7 (2009), which is hereby incorporated by reference in its entirety. These findings provide evidence for a role of CLDN1 variants in AD and its associated phenotypes, further supporting the importance of CLDN1 in AD.

This study provides the first evidence that epidermal TJs are defective in AD, the most common human skin disease. It was observed that claudin-1 is selectively reduced in the epidermis of AD patients and that CLDN1 may be a novel AD susceptibility gene. Epidermal samples from AD subjects had remarkable defects in resistance and ion transport compared to healthy controls. Using the model of human keratinocyte monolayers, it was observed enhanced claudin-1 expression and recruitment to intercellular junctions, upon cell differentiation, which coincided with the development of a paracellular barrier. Selective downregulation of claudin-1 expression markedly increased paracellular permeability, decreased resistance and enhanced proliferation indicative of a wound repair response. The reduced expression of claudin-1 in AD epidermis may enhance the penetration of many relevant environmental antigens leading to greater allergen sensitization as well as greater susceptibility to irritants/pollutants and possibly even altered microbial flora. The inverse relationship between claudin-1 and serum total IgE values also indicates that this defect may promote Th2 responses. Collectively, this data indicates that barrier dysfunction in subjects with AD extends beyond the stratum corneum to TJ, the second barrier structure, and that barrier regulation provides a novel therapeutic opportunity in AD and possibly other atopic disorders.

### Materials and Methods for Example 8

### Culture of Primary Human Foreskin Keratinocytes (PHK):

Human keratinocytes were isolated from neonatal foreskin, as described above. PHK were cultured in Keratinocyte-SFM (Invitrogen/Gibco) with 1% Pen/Strep, 0.2% Amphotericin B (Invitrogen/Gibco). To differentiate PHK, cells were grown in DMEM (Invitrogen/Gibco) with 10% heat-inactivated fetal bovine serum (Invitrogen/Gibco) and 1% Pen/Strep, 0.2% Amphotericin B (Invitrogen/Gibco).

### RNA Interference:

PHK were plated on glass coverslips at 2−3×10⁵ cells/well in a E-well plate or at 2−3×10⁴ cells/filter in Transwell inserts (Costar; PET membrane, 0.4 μm pore size, 6.5 mm insert) in Keratinocyte-SFM without antibiotics. The next day after plating, cells were transfected with claudin-1 specific or control (scrambled) siRNAs (Santa Cruz) using Lipofectamine™ 2000 transfection Reagent (Invitrogen).

### HSV-1 Infection of PHK:

Infection studies were performed using the highly virulent HSV-1 strain F (provided by Dr. D.C. Johnson). Twenty-four to 48 h post-transfection with 100 nM claudin-1 or control siRNA, the cells were differentiated in DMEM with 10% heat-inactivated FBS for 24 h. Cells were washed twice with HBSS and infected with HSV-1 strain F at a multiplicity of infection (MOI) of 0.1 in DMEM containing 1% heat-inactivated FBS at 37° C., with rocking every 15 min. After 2 h, the viral inoculum was removed, and the cells were washed 2× with HBSS and incubated in DMEM containing 5% HI-FBS and 0.4% human-γ-globulin (Sigma; final concentration 0.5 mg/ml) for 24 hr to neutralize any extracellular virus.

### HSV-1 Fluorescent-Focus Assay:

HSV-infected PHK were washed 2× with PBS and fixed in 4% formaldehyde/PBS for 20 min at room temperature. A polyclonal rabbit anti-HSV-1 (Dako) antibody diluted 1:500 in PBS/1% BSA was placed on the PHK for 1 h at 37° C. followed by Alexa Fluor 488 donkey-anti-rabbit IgG H+L (1:1000, Molecular Probes) and 4′,6-diamidino-2-phenyl-indole, dihydrochloride (DAPI) (1:10,000, Molecular Probes). Coverslips were mounted onto slides with SlowFade (Molecular Probes). For each sample, six random fields were captured at identical acquisition settings. Images were stored in Portable Network Graphics (PNG) format and analyzed computationally to objectively quantify differences in focal forming units (FFU). HSV-1 infected cells were assigned to the green channel. Cell density was calculated by counting the number of DAPI labeled nuclei, assigned to the blue channel. Images were analyzed using MATLAB to enumerate FFU, and total cell number. The following FFU measurements were also taken: Major Axis Length (μm), Area (μm²), and Pixel Area (px).

The infected cell channel (green) was converted to a binary image using the Otsu method for threshold determination. Then a morphological closing (dilation followed by erosion) was done to reduce noise and fuse individual infected cells into colonies. Then a distance transform was computed from the binary image, followed by a watershed transform. The colony binary image was then multiplied by its watershed image, the result was dilated slightly, and its perimeter was drawn. Only cells with sizes greater than a predefined threshold were included for further analysis. The infected cell channel (green) was filtered to reduce effects of noise. The resulting image was inverted, followed by a watershed transform. The infected cell watershed image was then multiplied by the colony watershed image to identify cells within previously identified colonies. Finally, a perimeter was drawn around cell borders.

### Infectious Center Assay:

Twenty-four hours post-infection, PHK were treated with 0.05% Trypsin—EDTA (Invitrogen/Gibco) for 5 min at 37° C. followed by gently scraping to lift the cells, washed 3 times in DMEM containing 1% HI-FBS, and vortexed for 30 sec to disrupt cell clumps. Alive cells were counted using Trypan blue exclusion and scalar PHK dilutions (1000 to 1 cells in 1 ml of media) were plated onto pre-grown monolayers of Vero cells in six well plates. After 2 h, 1 ml of DMEM containing 5% HI-FBS and 0.4% human-γ-globulin (Sigma; final concentration 0.5 mg/ml) was added to each well. After 3 days incubation at 37° C., cells were fixed (75% MeOH/25% acetic acid) and stained with 0.1% crystal violet. Plaques were counted by 2 independent investigators and results expressed as percent of infected PHK.

Real-Time PCR (qPCR):

Reverse transcription was performed from total RNA using iScript™ cDNA Synthesis kit (Bio-Rad) according to the manufacturer's protocol. qPCR was performed using the iQ™ SYBER Green Supermix assay system from Bio-Rad Laboratories. All PCR amplifications were carried out in triplicate on an iQ5 Multicolor real-time PCR detection system (Bio-Rad). Primers were designed and synthesized by Integrated DNA Technologies. Primers used in the study: GAPDH (forward: SEQ ID NO: 16 and reverse: SEQ ID NO: 17); Cldn-1 (forward: (SEQ ID NO:18 and reverse: SEQ ID NO:19); PVRL1 (Nectin1; forward: SEQ ID NO:28 and reverse SEQ ID NO:29).

Relative gene expression was calculated by using the T^{-ΔΔCt} method, in which Ct indicates cycle threshold, the fractional cycle number where the fluorescent signal reaches detection threshold. Livak and Schmittgen, “Analysis of Relative Gene Expression Data Using Real-Time Quantitative PCR and the 2(-Delta Delta C(T)) Method,” Methods 25:402-8 (2001), which is hereby incorporated by reference in its entirety. The normalized Ct value of each sample was calculated using GAPDH as an endogenous control gene.

### Genetic Study Participants:

DNA was isolated using standard protocols from 258 unrelated European American AD patients and 156 non-atopic healthy controls participating in the ADVN. The same set of markers was genotyped on 176 African American AD patients, and 152 non-atopic healthy controls (details can be found in previous publications such as Beck et al., “Phenotype of Atopic Dermatitis Subjects With a History of Eczema Herpeticum,” J. Allergy Clin. Immunol. 124:260-9, 9 e1-7 (2009), which is hereby incorporated by reference in its entirety). AD was diagnosed using the US consensus conference criteria. Eichenfield, L. F., “Consensus Guidelines in Diagnosis and Treatment of Atopic Dermatitis,” Allergy 59 Suppl 78:86-92 (2004), which is hereby incorporated by reference in its entirety. ADEH+ was defined as AD patients with at least one EH episode documented either by an ADVN investigator (or a physician affiliated with the same academic center) or diagnosed by another physician and confirmed by PCR, tissue immunofluorescence, Tzanck smear and/or culture. Nonatopic, healthy controls (NA) were defined as having no personal history of chronic disease including atopy. AD severity was defined according to the ‘eczema area and severity index’ (EAST), a standardized grading system (Hanifin et al., “The Eczema Area and Severity Index (EAST): Assessment of Reliability in Atopic Dermatitis. EASI Evaluator Group,” Exp. Dermatol. 10:11-8 (2001), which is hereby incorporated by reference in its entirety), and total serum IgE was measured. The study was approved by the institutional review boards at National Jewish Health, Johns Hopkins University, Oregon Health & Science University, University of California San Diego, Children's Hospital of Boston and University of Rochester Medical Center. All subjects gave written informed consent prior to participation.

### Genotyping and Quality Control:

Genotyping was performed as described above. Modest associations between CLDN1 SNPs and EH was observed in the North American populations. It was then questioned whether the strong association observed between both FLG null mutations R501X and 2282de14 and EH (OR=10.1 [4.7-22.1]; P=1.99×10¹¹) was masking a lesser effect from CLDN1 variants. Eichenfield, L.F., “Consensus Guidelines in Diagnosis and Treatment of Atopic Dermatitis,” Allergy 59 Suppl 78:86-92 (2004), which is hereby incorporated by reference in its entirety. To address this, subjects were excluded who had one or both of these FLG mutations and the associations were reanalyzed with all CLDN1 SNPs. In the European American group, an intronic SNP (rs3774032; OR=0.59 [0.35-0.98] P=0.037) was significantly associated with EH (FIG. 18). When excluding subjects with a FLG mutation (n=29 [36.3%]; Table 5), the association became slightly more significant (OR=0.44 [0.22-0.85], P=0.0225). Interestingly, one additional intronic SNP emerged (rs3732923, OR=1.93 [1.21-3.07], P=0.0010) (FIG. 18). Although the EH sample size in the African American population was too small (n=21) for meaningful analyses, a SNP in the promoter region was associated with EH (rs3954259; OR=2.16 [1.02-4.59], P=0.040), and this association was enhanced when subjects (n=2 [12.5%]; Table 5) with FLG mutations (OR=2.30 [1.01-5.28], P=0.026) were excluded. Limitations of these studies are the small sample size and the superficial coverage of CLDN1 loci. Nevertheless, these findings implicate CLDN1 mutations in susceptibility to widespread HSV-1 skin infections in subjects with AD, especially those who do not have a FLG mutation.

TABLE 5
ADVN Registry Participant Characteristics:

| | European American | | | | | | African American | | | | | |
|---|---|---|---|---|---|---|---|---|---|---|---|---|
| | | | | | Non- | | | | | | Non- | |
| CHARACTERISTIC | ADEH+ | | ADEH− | | Atopic | | ADEH+ | | ADEH− | | Atopic | |
| Sample size | 93 | | 165 | | 156 | | 21 | | 155 | | 152 | |
| Males; N | 49 | (52.7%) | 47 | (28.5%) | 63 | (40.4%) | 8 | (38.1%) | 35 | (22.6%) | 77 | (50.7%) |
| (%) | | | | | | | | | | | | |
| Age; mean | 22.9 | (21.5) | 38.0 | (14.5) | 36.6 | (13.2) | 22.8 | (19.0) | 36.4 | (11.0) | 41.1 | (10.3) |
| (SD) | | | | | | | | | | | | |
| AD onset <5 yrs; | 77 | (97.5%) | 101 | (78.3%) | NA | | 14 | (93.3%) | 77 | (68.1%) | NA | |
| N (%) | | | | | | | | | | | | |
| Median IgE | 2225 | (611-9034) | 252 | (85-1275) | 52.1 | (17.6-154.8) | 3505 | (571-9429) | 425 | (129-1194) | 152.9 | (48.8-405.6) |
| levels; | | | | | | | | | | | | |
| (Interquartile | | | | | | | | | | | | |
| Range)* | | | | | | | | | | | | |
| Median EASI^{†}; | 10.0 | (4.4-15.8) | 3.2 | (1.3-8.4) | NA | | 10.9 | (6.8-15.8) | 3.0 | (1.5-7.4) | NA | |
| (Interquartile | | | | | | | | | | | | |
| Range)** | | | | | | | | | | | | |
| FLG Null | 29 | (36.3%) | 36 | (22.1%) | 9 | (5.8%) | 2 | (12.5%) | 9 | (6.0%) | 2 | (1.3%) |
| Allele | | | | | | | | | | | | |
| Carrier; | | | | | | | | | | | | |
| N (%) | | | | | | | | | | | | |

The following abbreviations are used in the above table: AD, atopic dermatitis; ADEH+, atopic dermatitis with a history of eczema herpeticum; ADEH−, AD without a history of EH; EASI, Eczema area and severity index; and NA, not applicable.
^{†}EASI determined by the percentage of eczema area on a 7-point ordinal scale: 0 = no eruption; 1 = <10%; 2 = 10%-29%; 3 = 30%-49%; 4 = 50%-69%; 5 = 70%-89%; and 6 = 90%-100%.
*Total serum IgE levels were significantly higher in ADEH+ patients compared to both ADEH− patients and non-atopic, healthy controls even after adjusting for age (P < 0.001).
**EASI was significantly higher among ADEH+ patients compared to ADEH− patients in both racial groups even after adjusting for age (P < 0.001).

### Example 8

### Claudin-1 Defect in Atopic Dermatitis May Enhance Susceptibility to HSV-1 Infections

Viruses often commandeer intercellular junction proteins as a means of viral entry. Niessen, C. M., “Tight Junctions/Adherens Junctions: Basic Structure and Function,” J. Invest. Dermatol. 127:2525-32 (2007), which is hereby incorporated by reference in its entirety. For example, wild-type HSV-1 infects keratinocytes through a complex interaction involving the viral envelope glycoprotein gD with either nectin-1 (PVRL1) or herpes virus entry mediator (HVEM). Gonzalez-Mariscal et al., “Virus Interaction With the Apical Junctional Complex,” Front. Biosci. 14:731-68 (2009); Geraghty et al., “Entry of Alphaherpesviruses Mediated by Poliovirus Receptor-Related Protein 1 and Poliovirus Receptor,” Science 280:1618-20 (1998), which are hereby incorporated by reference in their entirety. Nectin-1, a Ca²-independent cell adhesion protein of the immunoglobulin superfamily, co-localizes with E-cadherin and catenin to form another intercellular junctional complex called adherens junctions. Previous studies have shown that the susceptibility of human keratinocytes to HSV-1 infection is inversely related to the degree of cell-cell contact formation and confluency. Geraghty et al., “Entry of Alphaherpesviruses Mediated by Poliovirus Receptor-Related Protein 1 and Poliovirus Receptor,” Science 280:1618-20 (1998); Schelhaas et al., “Herpes Simplex Virus Type 1 Exhibits a Tropism for Basal Entry in Polarized Epithelial Cells,” J. Gen. Virol. 84:2473-84 (2003), which are hereby incorporated by reference in their entirety. Because intercellular junctions only develop in confluent cell cultures, this suggests that healthy junctional complexes may be a key deterrent to the spread of HSV from one keratinocyte to another.

Atopic Dermatitis subjects are recognized for their susceptibility to recurrent, widespread cutaneous infections with HSV-1, called eczema herpeticum (“EH”). In these studies, the hypothesis that the silencing of epidermal CLDN1 will enhance HSV-1 infectivity of human keratinocytes was tested. In the preceding Examples, it was demonstrated that reductions in CLDN1 adversely affected measures of TJ integrity. Primary human keratinocytes (PHK) were isolated from foreskin as previously described (De Benedetto et al., “Tight Junction Defects in Atopic Dermatitis,” J. Allergy Clin. Immunol. (10.1016/j.jaci.2010.10.018) (2010), which is hereby incorporated by reference in its entirety) and transiently transfected with scrambled (control) or CLDN1 (100 nM) siRNA (Santa Cruz Biotechnology, Inc; CA) using Lipofectamine™ 2000 transfection reagent (Invitrogen, Carlsbad, Calif.). Twenty-four to 48 h post-transfection, PHK were differentiated in DMEM with 10% heat-inactivated fetal bovine serum (HI-FBS; Invitrogen/Gibco) for 24 h and subsequently infected with the highly virulent HSV-1 strain F (provided by Dr. D.C. Johnson) at a multiplicity of infection (MOI) of 0.1.

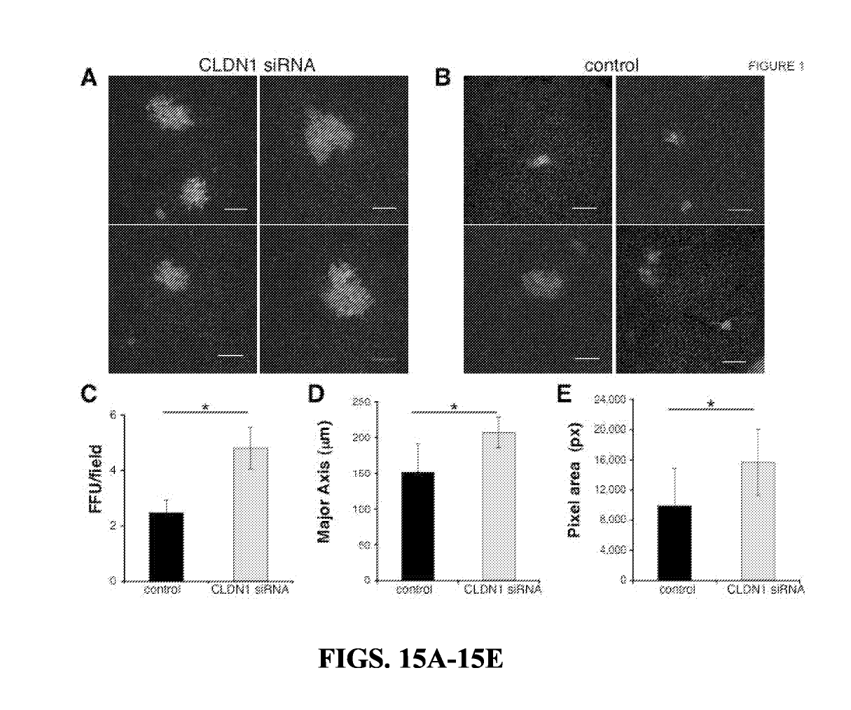

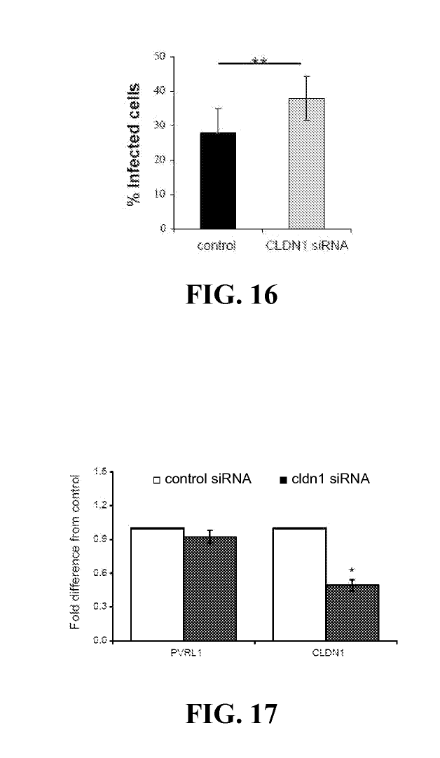

The MOI was chosen based on findings from viral titration (0.01 to 10 MOI) studies on differentiated and undifferentiated PHK, respectively, used as negative and positive controls. After 2 h, the viral inoculum was removed and PHK were cultured for 24 h in DMEM containing 5% HI-FBS and 0.4% human-γ-globulin (0.5 mg/ml; Sigma, St. Louis, Mo.) to neutralize extracellular virus. HSV-infected PHK were fixed in 4% formaldehyde and stained with a polyclonal rabbit anti-HSV-1 (1:500; Dako, Carpinteria, Calif.) antibody followed by Alexa Fluor 488 donkey-anti-rabbit IgG H+ L (1:1000, Invitrogen/Molecular probe) and 4′,6-diamidino-2-phenyl-indole, dihydrochloride (DAPI; 1:10,000, Molecular Probes). For each sample, six random fields were captured at identical acquisition settings and analyzed computationally to objectively quantify differences in focal forming units (FFU). A FFU was defined as a cluster of 3 or more adjacent HSV-1 positive cells. Claudin-1 depletion significantly increased the number and size of HSV-1 FFU (FIG. 15A) as compared to control siRNA transfected PHK (FIGS. 15B, 15C). An average of 4.8±0.7 FFU/field were counted in CLDN1 knockdown PHK, while only 2.5±0.5 FFU/field were detected in the control cells (P=0.05; n=6; FIG. 15C). Adapted Fluorospot software was used to semi-quantify differences in dimensions of each FFU. The diameters of FFU were significantly greater in keratinocytes whose CLDN1 expression was reduced (207±22 μm) compared to controls (152±40 μm; P=0.03; n=6; FIG. 15D). Similar findings were observed when evaluating FFU area (P=0.04; n=6; FIG. 1E) in PHK after CLDN1 knockdown (15649±4367 pixels) as compared to control transfection (9902±4943 pixels). The increased frequency of HSV-1-infected cells in CLDN-1 knockdown PHK was confirmed by an infectious center assay. More CLDN1 siRNA transfected PHK were infected with HSV-1 (38±6%; n=3) than control PHK (28±7%; n=3; P=0.003) (see FIG. 16). Importantly, the observed increase in HSV-1 infectiveness in CLDN1 knockdown PHK could not be explained by reduced expression of nectin-1, the HSV-1 receptor. Indeed, nectin-1 mRNA expression was not affected by CLDN1 silencing (n=5; see FIG. 17). However, the possibility cannot be ruled out that reductions in CLDN1 and the affects on TJ function might reduce nectin-1 membrane localization.

As part of the NIAID-funded Atopic Dermatitis and Vaccinia Network (ADVN), a preliminary study was performed to evaluate whether CLDN1 polymorphisms were associated with EH. To do this, a haplotype-tagging SNP approach using genetic markers available in the public arena (n=132 in dbSNP) in 414 European Americans and 328 African Americans was used (see FIG. 10). Example 7 and the discussion of Examples 1-7, above, illustrate modest associations (P=0.003-0.05) between SNP variants throughout the CLDN1 gene and AD that were more significant among African Americans. In this Example, the question of whether genetic variations in CLDN1 contribute to risk of EH in the same North American populations was addressed. The EH subphenotype was defined as AD subjects that had at least one EH episode documented either by an ADVN investigator (or a physician affiliated with the same academic center) or diagnosed clinically by an outside physician where the HSV infection was confirmed by PCR, tissue immunofluorescence, Tzanck smear and/or culture. Nonatopic, healthy controls (NA) were defined as subjects having no personal history of chronic disease including atopic disorders.

This Example shows that the susceptibility of AD subjects to widespread cutaneous infections with HSV-1 and possibly other viral or bacterial pathogens might be due to epidermal barrier defects. Earlier work suggested that FLG mutations are associated with the phenotype of EH. In this Example, it is shown both mechanistic and genetic results that implicate tight junctions as a critical barrier structure as well, showing that reductions in claudin-1 levels may promote the spread of epidermal viral or bacterial infections.

### Example 9

### siRNA can Blunt Claudin-1 Expression in Treated Keratinocytes

siRNA approach was used to selectively downregulate claudin-1 in primary human keratinocytes (PHK). Claudin-1 (Cldn1) siRNA (100 nM) resulted in a 50% reduction (MEAN±SEM: 5.42±0.8 RVU) in claudin-1 transcripts compared to control transfected cells (11.8±2 RVU; *P=0.009, n=5/group). There was no effect on mRNA expression of occludin, ZO-1, nectin-1, E-cadherin or filaggrin (n=5/group). Relative gene expressions were calculated by using the 2^{-ΔΔCt} method, in which Ct indicates cycle threshold, the fractional cycle number where the fluorescent signal reaches detection threshold. The normalized Ct value of each sample was calculated using GAPDH as endogenous control gene. The specific sequence information for the claudin-1 siRNA (h) is detailed below. (This siRNA is a pool of 3 separate strands.)

mRNA accession 3′ to 5′: NM_{—}021101

Sense Strand(A):
(SEQ ID NO: 10)
UACAUAGGCAUAGUUCAUGtt
(SEQ ID NO: 11)
CAUGAACUAUGCCUAUGUAtt
Sense Strand(B):
(SEQ ID NO: 12)
AACGUAUGCAGUUAAUUCCtt
(SEQ ID NO: 13)
GGAAUUAACUGCAUACGUUtt
Sense Strand(C):
(SEQ ID NO: 14)
UGAAGAUCUAUGUAUGUGGtt
(SEQ ID NO: 15)
CCACAUACAUAGAUCUUCAtt

Depletion of claudin-1 significantly reduced trans epithelial electric resistance (TEER; control: 164±18.2 and Cldn1 siRNA: 80.6±6.4 ohms×cm²; P=0.007; N=4) and increased sodium fluorescein flux (control: 27.6±11.7 and Cldn1 siRNA: 52.5±9.2; P=0.026; N=4) when compared to the scrambled RNA-transfected PHK.

### Example 10

### S. aureus-Related Ligands Increase TJ Function in Primary Human Keratinocytes

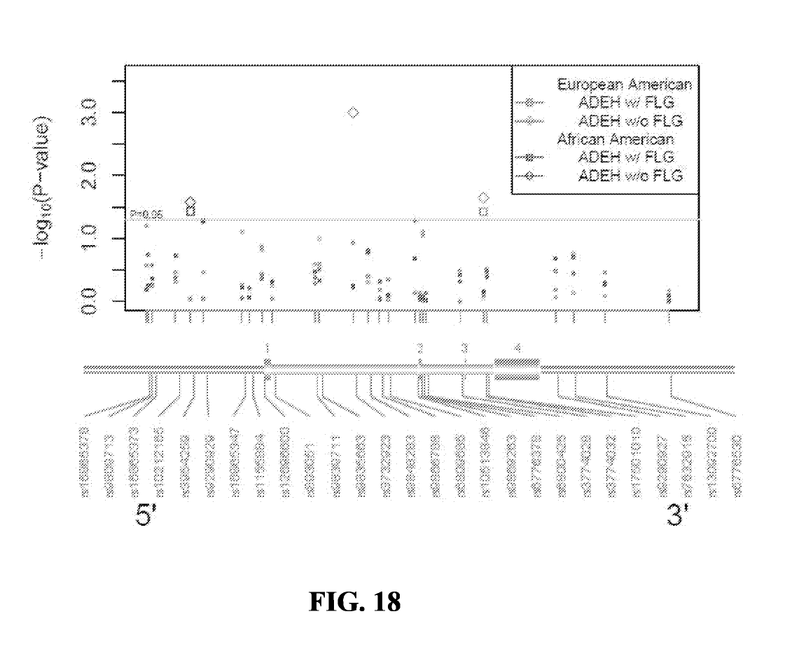

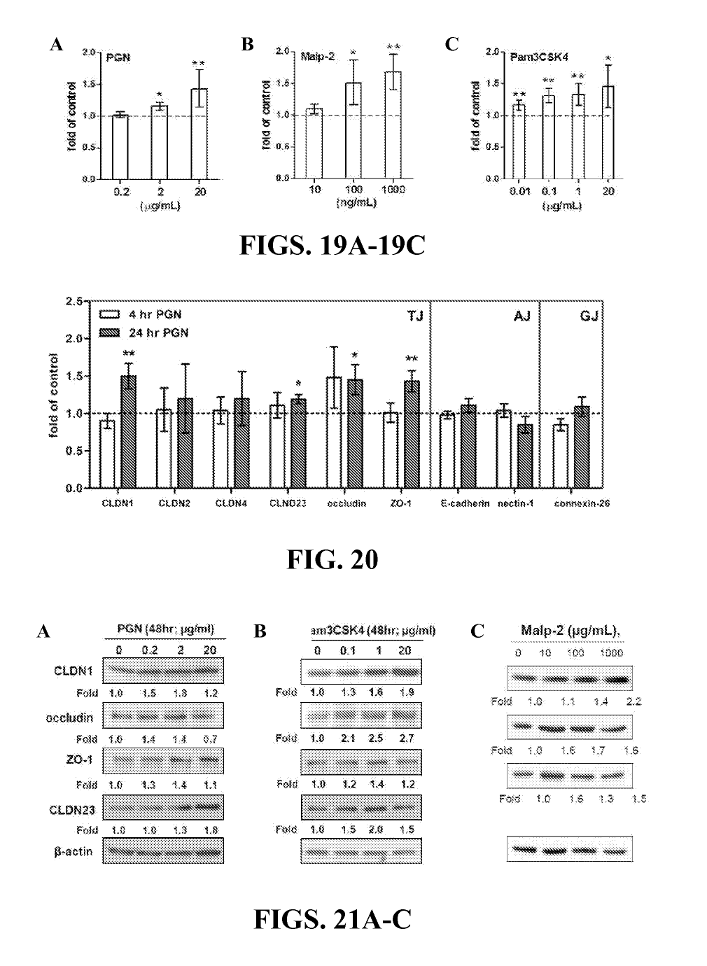

TEER was measured in PHK as described above. PHK were stimulated with S. aureus-derived peptidoglycan (PGN, 0.2-20 μg/ml, n=3-7) (FIG. 19A), Malp-2 (10-1000 ng/ml, n=3) (FIG. 19B), and Pam3CSK4 a TLR1/2 ligand (0.01-20 μg/ml, n=3-6) (FIG. 19C). Data are presented by analyzing the area under the TEER curve and normalized to the mean values for the control group (media alone)*p<0.05; **p<0.01. This data demonstrates that these S. aureus-related ligands increase tight junction function in primary human keratinocytes.

S. aureus-derived PGN also increases TJ mRNA expressions in primary human keratinocytes. The mRNA expression of tight junction molecules (CLDN1, CLDN2, CLDN4, CLDN23, occludin, ZO-1), gap junction molecule (connexin-26, connexin-43), adherens junction molecules (nectin-1, E-cadherin), and desmosome molecules (DSG-1, DSG-3) were quantified by qPCR before and two timepoints (4 and 24 hr) after stimulation with PGN (20 μg/ml) in PHFK (n=4-7) and the results are shown in FIG. 20. The dotted line represents expression levels for the control group (media alone) at each timepoint. *p<0.05; **p<0.01.

S. aureus-derived PGN and TLR2 ligand both increase TJ protein expressions in primary human keratinocytes. PHFK were treated with PGN (0, 0.2, 2, 20 μg/ml) (as shown in FIG. 21A), Pam3CSK4 (0, 0.1, 1, 20 μg/ml) (as shown in FIG. 21B), MALP2 (0, 10, 100, 1000 ng/ml) for 48 hr and CLDN1 (as shown in FIG. 21C), occludin, ZO-1 and CLDN23 protein levels were detected from whole cell lysates by Western blot. Quantitative protein expression was determined by densitometry of bands and normalization to the housekeeping protein (13-actin). (Representative blot of n=2-3 experiments).

### Example 11

### PPARγ Agonists Upregulate Epidermal TJ

Several PPARs mediated biological activities have been demonstrated in human skin and primary keratinocytes culture, including anti-proliferative, pro-differentiative and anti-inflammatory properties (see Kuenzli and Saurat, “Peroxisome Proliferator-Activated Receptors as New Molecular Targets in Psoriasis,” Current Drug Targets—Inflammation & Allergy 3(2):205-211 (2004), which is hereby incorporated by reference in its entirety). In vitro studies have also shown PPARγ agonists upregulated the TJ barrier function in several human epithelial cells (see Varley et al., “PPARγ-Regulated Tight Junction Development During Human Urothelial Cyto Differentiation,” J. Cell. Physiol. 208(2):407-17 (2006); Huang et al., “PPARalpha and PPARgamma Attenuate HIV-induced Dysregulation of Tight Junction Proteins by Modulations of Matrix Metalloproteinase and Proteasome Activities,” FASEB J. 23(5):1596-606 (2009); Ogasawara et al., “PPARgamma Agonists Upregulate the Barrier Function of Tight Junctions via a PKC Pathway in Human Nasal Epithelial Cells,” Pharmacol Res. 61(6):489-98 (2010), which are hereby incorporated by reference in their entirety).

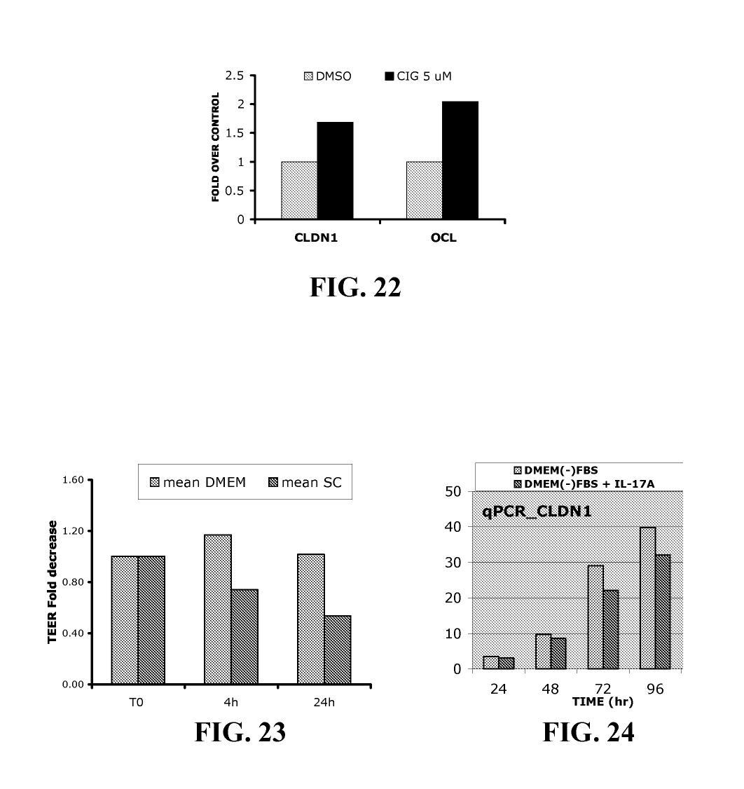

In vitro experiments were performed using primary human keratinocytes (“PHK”) and found that Ciglitazone (CIG, 5 μM, Cayman Chemical; 24 h) enhanced claudin-1 and occludin expression (FIG. 22). FIG. 22 shows the results, that treatment with CIG 5 μM enhanced CLDN1 mRNA (1.7 fold over DMSO alone) and occludin mRNA (2 fold) expression in PHK differentiated in high Calcium media (DMEM) for 24 h.

PHK were differentiated in high Calcium media (DMEM) in the presence of CIG 5 μM in DMSO or media with equivalent amount of DMSO alone, for 24 h. Total RNA was extracted from PHK using the QlAshredder spin column (Qiagen) and RNeasy RNA isolation kits (Qiagen). The quality of total RNA samples (RNA Integrity Number, RIN) was assessed using an Agilent 2100 Bioanalyzer (Agilent Technologies, Palo Alto, Calif.).

qPCR was performed using the iScript™ cDNA Synthesis kit and iQ™ SYBER Green Supermix assay system (Bio-Rad Laboratories). All PCR amplifications were carried out in triplicate on an iQ5 Multicolor real-time PCR detection system (Bio-Rad). Primers were designed and synthesized by Integrated DNA Technologies. Relative gene expression was calculated by using the 2^{-ΔΔCt} method, as previously described. The normalized Ct value of each sample was calculated using GAPDH as an endogenous control gene.

### Example 12

### Sodium Decanoate (Caprate) Reduces Human Epidermal Barrier Function

Previously, it has been shown that sodium caprate (C10) treatment decreased transepithelial electrical resistance and increased paracellular permeability of human cultured keratinocytes and reconstructed human epidermis. Kurasawa et al., “Regulation of Tight Junction Permeability by Sodium Caprate in Human Keratinocytes and Reconstructed Epidermis,” Biochem. Biophys. Res. Commun. 381(2):171-5 (2009), which is hereby incorporated by reference in its entirety. These findings were extended showing TJ disruption in intact human epidermis.

Epidermal sheets were obtained from discarded human skin using a weck blade (0.08 inch). Epidermal samples were mounted in an adapted snap-well system (Pierro et al., “Zonula Occludens Toxin Structure-Function Analysis, Identification of the Fragment Biologically Active on Tight Junctions and of the Zonulin Receptor Binding Domain,” J. Biol. Chem. 276 (22):19160-5 (2001), which is hereby incorporated by reference in its entirety.) and equilibrated in DMEM (Gibco) before measuring TEER using an EVOMX voltohmmeter (World Precision Instruments, Sarasota, Fla.). Sodim decanoate (Sigma; 1002-62-6) 1 mM in DMEM or media alone was added to the upper well and TEER measured, as described above, 4 and 24 hours after. Results are shown in Table 6 (below) and FIG. 23.

TABLE 6
Affect of Sodium Decanoate on TEER

| | | | mean | dmem + | dmem + | mean |
|---|---|---|---|---|---|---|
| time | DMEM_1 | DMEM_2 | DMEM | SC_1 | SC_2 | SC |
| T0 | 1.00 | 1.00 | 1.00 | 1.00 | 1.00 | 1.00 |
| 4 h | 1.11 | 1.22 | 1.17 | 0.79 | 0.69 | 0.74 |
| 24 h | 1.11 | 0.92 | 1.02 | 0.56 | 0.51 | 0.54 |

### Example 13

### Treatment with IL17 (100 ng/ml) Reduced CLDN1 mRNA Expression in PHK Differentiated in High Calcium Media (DMEM)

PHK were differentiated in high Calcium media (DMEM) in the presence of human recombinant IL17 (R&D system) or media alone for up to 96 hours. Media was changed every 48 h. Total RNA was extracted from PHK using the QlAshredder spin column (Qiagen) and RNeasy RNA isolation kits (Qiagen). The quality of total RNA samples (RNA Integrity Number, RIN) was assessed using an Agilent 2100 Bioanalyzer (Agilent Technologies, Palo Alto, Calif.).

qPCR was performed using the iScript™ cDNA Synthesis kit and iQ™ SYBER Green Supermix assay system (Bio-Rad Laboratories). All PCR amplifications were carried out in triplicate on an iQ5 Multicolor real-time PCR detection system (Bio-Rad). Primers were designed and synthesized by Integrated DNA Technologies. Relative gene expression was calculated by using the 2^{-ΔΔCt} method.

Reduced expression of CLDN1 was observed at each time point between 24 and 96 h (see FIG. 24). FIG. 24 shows a representative experiment of n=2. Table 7, below, shows data that is expressed as claudin1 RNA fold change in IL17 vs. media alone treated cells for each donor and as mean±SEM.

TABLE 7
CLAUDIN1 fold decrease IL17/media alone

| Time (hr) | donor#1 | donor#2 | mean (1 and 2) |
|---|---|---|---|
| 24 | 0.87 | 1.09 | 0.98 |
| 48 | 0.88 | 0.66 | 0.77 |
| 72 | 0.76 | 0.46 | 0.61 |
| 96 | 0.81 | 0.86 | 0.83 |

These results show that treatment with IL17 (100 ng/ml) reduced CLDN1 mRNA expression in PHK differentiated in high Calcium media (DMEM).

Although the invention has been described in detail for the purpose of illustration, it is understood that such detail is solely for that purpose, and variations can be made therein by those skilled in the art without departing from the spirit and scope of the invention which is defined in the following claims. Further, even though specific combinations may not be explicitly recited herein, it is to be understood by persons of skill in the art that such combinations of features are intended to be encompassed by the present disclosure for the recited products and methods.

What is claimed:

1. A transdermal vaccine formulation comprising:
a pharmaceutically suitable carrier;
an effective amount of an antigen or antigen-encoding nucleic acid molecule present in the carrier, and optionally one or more adjuvants; and
an amount of IL-17 that is sufficient to transiently disrupt claudin-1 and/or -23 function within tight junctions.

2. The transdermal vaccine formulation according to claim 1, wherein the formulation comprises an antigen selected from the group of an allergen, an immunogenic subunit derived from a pathogen, a virus-like particle, or an attenuated virus particle.

3. The transdermal vaccine formulation according to claim 1, wherein the formulation comprises an antigen-encoding nucleic acid molecule.

4. The transdermal vaccine formulation according to claim 3, wherein the antigen is selected from the group of an allergen, an immunogenic subunit derived from a pathogen, a virus-like particle, or glycoprotein or glycolipid conjugated to an immunogenic polypeptide.

5. The transdermal vaccine formulation according to claim 1, wherein the carrier comprises one or more of sugar, polylysine, polyethylenimine, polyethylenimine derivatives, and liposomes, together with their derivatives.

6. The transdermal vaccine formulation according to claim 1, wherein the carrier is a mannosylated polyethylenimine.

7. The transdermal vaccine formulation according to claim 1, wherein the carrier comprises a solution or emulsion that is substantially free of inorganic salt ions and includes one or more water soluble or water-emulsifiable substances capable of making the vaccine isotonic or hypotonic, and the adjuvant is a polycation optionally modified with a sugar group.

8. The transdermal vaccine formulation according to claim 1, wherein the carrier comprises a solution or emulsion, and the adjuvant is combination of a polycation and an immunostimulatory CpG or non-CpG oligodeoxynucleotide.

9. The transdermal vaccine formulation according to claim 1, wherein the formulation does not include an adjuvant.

10. A transdermal drug formulation comprising:
a pharmaceutically suitable carrier;
an effective amount of a therapeutic agent; and
an amount of IL-17 that is sufficient to transiently disrupt claudin-1 and/or -23 function within tight junctions.

11. The transdermal drug formulation according to claim 10, wherein the therapeutic agent is selected from a group consisting of antiinfectives, antibiotics, antiviral agents, analgesics, fentanyl, sufentanil, buprenorphine, analgesic combinations, anesthetics, anorexics, antiarthritics, antiasthmatic agents, terbutaline, anticonvulsants, antidepressants, antidiabetic agents, antidiarrheals, antihistamines, antiinflammatory agents, antimigraine preparations, antimotion sickness, scopolamine, ondansetron, antinauseants, antineoplastics, antiparkinsonism drugs, cardiostimulants, dobutamine, antipruritics, antipsychotics, antipyretics, antispasmodics, gastrointestinal and urinary, anticholinergics, sympathomimetics, xanthine derivatives, cardiovascular preparations, calcium channel blockers, nifedipine, beta-blockers, beta-agonists, salbutamol, ritodrine, antiarrythmics, antihypertensives, atenolol, ACE inhibitors, diuretics, vasodilators, coronary, peripheral and cerebral, central nervous system stimulants, cough and cold preparations, decongestants, diagnostics, hormones, parathyroid hormone, growth hormone, insulin, hypnotics, immunosuppressives, muscle relaxants, parasympatholytics, parasympathomimetics, anti-oxidants, nicotine, prostaglandins, psychostimulants, sedatives, tranquilizers, skin acting anti-oxidants, caretenoids, ascorbic acid (vitamin C), vitamin E, anti wrinkling agents, retinoids, retinol (vitamin A alcohol), alpha-hydroxic acids, beta-hydroxy acid, salicylic acid, combination-hydroxy acids and poly-hydroxy acids, and hydrolyzed and soluble collagen, moisturizers, hyaluronic acid, anticellulite agents, aminophyllines, skin bleaching agents, retinoic acid, hydroquinone, peroxides, botanical preparations, extracts of aloe-vera, wild yam, hamamelitanin, ginseng, witch hazel, water, green tea, and combinations thereof.

12. The transdermal drug formulation according to claim 10, wherein the therapeutic agent is greater than 300 daltons.

13. The transdermal drug formulation according to claim 10, wherein the carrier is selected from the group consisting of tromethane ethanol, polyethylene glycol, glycerin, propylene glycol, acrylates, Carbopol, purified water, benzyl alcohol, cetyl alcohol, citric acid, monoglycerides, diglycerides, triglycerides, oleyl alcohol, sodium cetostearylsulphate, sodium hydroxide, stearyl alcohol, white petrolatum, mineral oil, propylene carbonate, white wax, paraffin, and any combination thereof.

14. A transdermal patch comprising the transdermal vaccine formulation of claim 1 or the transdermal drug formulation of claim 11.

15. The transdermal patch according to claim 14, further comprising
a backing material;
an adhesive material in contact with a first portion of the backing material; and
a drug or vaccine storage material comprising the transdermal drug or vaccine formulation, wherein the storage material is in contact with a second portion of the backing material.

16. The drug delivery patch of claim 15, wherein the therapeutic agent is selected from a group consisting of antiinfectives, antibiotics, antiviral agents, analgesics, fentanyl, sufentanil, buprenorphine, analgesic combinations, anesthetics, anorexics, antiarthritics, antiasthmatic agents, terbutaline, anticonvulsants, antidepressants, antidiabetic agents, antidiarrheals, antihistamines, antiinflammatory agents, antimigraine preparations, antimotion sickness, scopolamine, ondansetron, antinauseants, antineoplastics, antiparkinsonism drugs, cardiostimulants, dobutamine, antipruritics, antipsychotics, antipyretics, antispasmodics, gastrointestinal and urinary, anticholinergics, sympathomimetics, xanthine derivatives, cardiovascular preparations, calcium channel blockers, nifedipine, beta-blockers, beta-agonists, salbutamol, ritodrine, antiarrythmics, antihypertensives, atenolol, ACE inhibitors, diuretics, vasodilators, coronary, peripheral and cerebral, central nervous system stimulants, cough and cold preparations, decongestants, diagnostics, hormones, parathyroid hormone, growth hormone, insulin, hypnotics, immunosuppressives, muscle relaxants, parasympatholytics, parasympathomimetics, anti-oxidants, nicotine, prostaglandins, psychostimulants, sedatives, tranquilizers, skin acting anti-oxidants, caretenoids, ascorbic acid (vitamin C), vitamin E, anti wrinkling agents, retinoids, retinol (vitamin A alcohol), alpha-hydroxic acids, beta-hydroxy acid, salicylic acid, combination-hydroxy acids and poly-hydroxy acids, and hydrolyzed and soluble collagen, moisturizers, hyaluronic acid, anticellulite agents, aminophyllines, skin bleaching agents, retinoic acid, hydroquinone, peroxides, botanical preparations, extracts of aloe-vera, wild yam, hamamelitanin, ginseng, witch hazel, water, green tea, and combinations thereof.

17. The drug delivery patch of claim 15, further comprising a pharmaceutically acceptable carrier selected from the group consisting of tromethane ethanol, polyethylene glycol, glycerin, propylene glycol, acrylates, Carbopol, purified water, benzyl alcohol, cetyl alcohol, citric acid, monoglycerides, diglycerides, triglycerides, oleyl alcohol, sodium cetostearylsulphate, sodium hydroxide, stearyl alcohol, white petrolatum, mineral oil, propylene carbonate, white wax, paraffin, and any combination thereof.

18. A method of disrupting an epidermal barrier comprising:
applying to an epidermal site an amount of IL-17 in a carrier that is effective to decrease claudin-1 and/or -23 expression in keratinocytes present at the site, thereby disrupting barrier formation at the epidermal site.


SEQUENCE LISTING (35 sequences)

SEQ ID NO: 1 - RNA, 19 nt, artificial
  RNAi target sequence specific for claudin 1
gcaaagcaccgggcagaua

SEQ ID NO: 2 - RNA, 21 nt, artificial
  RNAi sense sequence specific for claudin 1
auagacgggccacgaaacguu

SEQ ID NO: 3 - RNA, 21 nt, artificial
  RNAi anti-sense strand specific for claudin 1
cguuucguggcccgucuauuu

SEQ ID NO: 4 - RNA, 19 nt, artificial
  RNAi target sequence specific for claudin 1
gaacaguacuuugcaggca

SEQ ID NO: 5 - RNA, 21 nt, artificial
  RNAi sense sequence specific for claudin 1
acggacguuucaugacaaguu

SEQ ID NO: 6 - RNA, 21 nt, artificial
  RNAi anti-sense strand specific for claudin 1
cuugucaugaaacguccguuu

SEQ ID NO: 7 - RNA, 19 nt, artificial
  RNAi target sequence specific for claudin 1
uuucaggucuggcgacauu

SEQ ID NO: 8 - RNA, 21 nt, artificial
  RNAi sense sequence specific for claudin 1
uuacagcggucuggacuuuuu

SEQ ID NO: 9 - RNA, 21 nt, artificial
  RNAi anti-sense strand specific for claudin 1
aaaguccagaccgcuguaauu

SEQ ID NO: 10 - DNA, 21 bp, Artificial
  claudin-1 specific RNAi sense strand
uacauaggcauaguucaugnn

SEQ ID NO: 11 - DNA, 21 bp, Artificial
  claudin-1 specific RNAi sense strand
caugaacuaugccuauguann

SEQ ID NO: 12 - DNA, 21 bp, Artificial
  claudin-1 specific RNAi sense strand
aacguaugcaguuaauuccnn

SEQ ID NO: 13 - DNA, 21 bp, Artificial
  claudin-1 specific RNAi sense strand
ggaauuaacugcauacguunn

SEQ ID NO: 14 - DNA, 21 bp, Artificial
  claudin-1 specific RNAi sense strand
ugaagaucuauguauguggnn

SEQ ID NO: 15 - DNA, 21 bp, Artificial
  claudin-1 specific RNAi sense strand
ccacauacauagaucuucann

SEQ ID NO: 16 - DNA, 19 bp, artificial
  primer
gaaggtgaaggtcggagtc

SEQ ID NO: 17 - DNA, 20 bp, artificial
  primer
gaagatggtgatgggatttc

SEQ ID NO: 18 - DNA, 20 bp, artificial
  primer
cgatgaggtgcagaagatga

SEQ ID NO: 19 - DNA, 20 bp, artificial
  primer
ccagtgaagagagcctgacc

SEQ ID NO: 20 - DNA, 20 bp, artificial
  primer
cttgccatgcaaactctcaa

SEQ ID NO: 21 - DNA, 20 bp, artificial
  primer
ttctcctcttggcttctgga

SEQ ID NO: 22 - DNA, 20 bp, artificial
  primer
ctggcctacaggaatacaag

SEQ ID NO: 23 - DNA, 20 bp, artificial
  primer
cttgatgtgtgacaatttgc

SEQ ID NO: 24 - DNA, 19 bp, artificial
  primer
cggaaaacatgctacacac

SEQ ID NO: 25 - DNA, 19 bp, artificial
  primer
cccatttactggctggtat

SEQ ID NO: 26 - DNA, 20 bp, artificial
  primer
gtttaacgcattgcccagtt

SEQ ID NO: 27 - DNA, 20 bp, artificial
  primer
ggcctacaggggtttcaaat

SEQ ID NO: 28 - DNA, 19 bp, artificial
  primer
agccattaaggagaaacga

SEQ ID NO: 29 - DNA, 19 bp, artificial
  primer
ttcccaatttctctgctct

SEQ ID NO: 30 - DNA, 20 bp, artificial
  primer
cagaaagttttccaccaaag

SEQ ID NO: 31 - DNA, 20 bp, artificial
  primer
aaatgtgagcaattctgctt

SEQ ID NO: 32 - DNA, 19 bp, artificial
  primer
gagctgaaggaacttctgg

SEQ ID NO: 33 - DNA, 21 bp, artificial
  primer
gatccatgaagacatcaacca

SEQ ID NO: 34 - protein, 28 aa, Artificial
  soluble CLDN-1 peptide fragment consensus sequence
SCVSQSTGQXQCKVFDSLLNLSSTLQAT

SEQ ID NO: 35 - protein, 49 aa, artificial
  soluble CLDN-23 peptide fragment
PGWRLVKGFLNQPVDVELYQGLWDMCREQSSRERECGQTDQWGYFEAQP